(19) United States
(12) Patent Application Publication
(10) Pub. No.: US 2023/0165842 A1
(43) Pub. Date: Jun. 1, 2023

(54) NEW USES

(71) Applicant: AIVIVO LTD, Milton, Cambridge (GB)

(72) Inventors: Peyman GIFANI, Cambridge (GB); Yasaman KALANTARMOTAMEDI, Cambridge (GB); Gholamreza BIDKHORI, Cambridge (GB)

(21) Appl. No.: 17/920,722

(22) PCT Filed: Apr. 22, 2021
(86) PCT No.: PCT/GB2021/050978

§ 371 Date: Oct. 21, 2022

(30) Foreign Application Priority Data
Apr. 22, 2020 (GB) 2005890.5

(51) Int. Cl. A61K 31/4418; A61K 45/06; A61P 31/14

(52) U.S. Cl. CPC A61K 31/4418; A61K 45/06; A61P 31/14

(57) ABSTRACT

There is provided inter alia a compound selected from Compounds (1 to 203) and prodrugs thereof and pharmaceutically acceptable salts and solvates thereof for use in the treatment or prevention of a disease selected from the group consisting of a positive-sense single-stranded RNA vims ((+)ssRNA vims) infection, a disease associated with a positive-sense single-stranded RNA virus ((+)ssRNA vims) infection and lung inflammation.

### TECHNICAL FIELD

This invention relates to new therapeutic uses of known compounds, specifically new uses for the treatment or prevention of a disease selected from the group consisting of a positive-sense single-stranded RNA virus ((+)ssRNA virus) infection, a disease associated with a positive-sense single-stranded RNA virus ((+)ssRNA virus) infection and lung inflammation.

### BACKGROUND TO THE INVENTION

The invention takes advantage of phenotypic drug discovery for the rapid identification of suitable therapeutics for the treatment of a range of disease indications. Such an approach provides a useful orthogonal method to established target-driven drug discovery methods.

Background to RNA Viruses, Coronaviruses and COVID-19

Eight virus families whose members infect vertebrates are currently known to possess single-stranded, positive-sense RNA genomes: the families

Picornaviridae, Caliciviridae and Hepeviridae have non-enveloped capsids, whereas the families Flaviviridae, Togaviridae, Arteriviridae and Coronaviridae are characterized by enveloped capsids. Coronaviruses are members of the subfamily Coronavirinae and cause respiratory and intestinal infections in animals and humans. There are four genera of Coronaviruses—Alphacoronavirus, Betacoronavirus, Gammacoronavirus and Deltacoronavirus, although only the Alphacoroanvirus and Betacoronavirus are known to infect mammals. Until the SARS-CoV outbreak in 2002, and the MERS-CoV outbreak in 2012, coronaviruses were not known to be pathogenic (Cui et al, 2019). Both of these viruses cause severe respiratory syndrome in human, whilst the four remaining human coronaviruses (HCoV-NL62, HCoV-229E, HCoV-0043 and HKU1) induce only mild upper respiratory diseases in immunocompetent hosts, such as the common cold (Corman et al., 2018; Singhal, 2020; Cui et al, 2019). To date, no clinical treatments have been developed for any coronavirus.

Coronavirus Disease 2019 (COVID-19) is a novel respiratory disease caused by severe acute respiratory syndrome coronavirus 2 (SARS-CoV-2) and has recently triggered a global pandemic (Singhal, 2020). The majority of patients infected with COVID-19 exhibit mild symptoms, but approximately 15% develop severe pneumonia and 5% develop acute respiratory distress syndrome (ARDS), septic shock and/or multiple organ failure (Cao, 2020). The mortality rate of COVID-19 is approximately 3% to 7%, compared to a mortality rate of less than 1% from influenza. There are currently no clinical or preventative treatments available for COVID-19, current methods relying on symptomatic management and oxygen therapy, with patients displaying severe symptoms being treated with mechanical ventilation (Mehta et al, 2020).

SARS-CoV-2 consists of four main structural proteins; spike (S), envelope (E), membrane (M) and nucleocapsid (N). SARS-CoV-2 is thought to target cells by binding to angiotensin-converting enzyme 2 (ACE2) receptors through the S protein, and subsequent structural changes allow the viral genes to enter the host cells (Sanders et al. 2020). The purported viral lifecycle provides potential opportunities for anti-viral drug therapy. A number of clinical trials are ongoing for repurposed anti-viral drugs for treatment of COVID-19, but to date no effective treatments have been discovered.

It has been reported that the majority of patients displaying sever COVID-19 symptoms exhibit hyperinflation of pro-inflammatory cytokines, characterised as cytokine storm (Mehta et al, 2020). High levels of such cytokines can lead to shock and tissue damage, respiratory failure or multiple organ failure. A number of studies aiming to dampen inflammatory responses have been reported, with elevated levels of IL-6 being found to be an indicator of poor outcome in patients with pneumonia and ARDS derived from COVID-19 (Cao, 2020). The use of small molecule cytokine inhibitors, particularly at an early stage in treatment, may be useful in blocking cytokine storm pathways and in limiting inflammatory damage caused by an excessive immune response.

Potential standardised therapeutic treatments for COVID-19 may involve the use of anti-viral or anti-inflammatory approaches, either alone or in combination (Cao, 2020).

Alternative approaches to drug discovery other than well-established target-based drug discovery could potentially identify suitable candidates for the treatment of coronavirus infections and diseases associated with a subject being infected with a coronavirus.

Background to Inflammation and Inflammation in Lung

Inflammation is the body's response to insults, which include infection, trauma, and hypersensitivity. The inflammatory response is complex and involves a variety of mechanisms to defend against pathogens and repair tissue. In the lung, inflammation is usually caused by pathogens or by exposure to toxins, pollutants, irritants, and allergens. During inflammation, numerous types of inflammatory cells are activated. Each releases cytokines and mediators to modify activities of other inflammatory cells. Orchestration of these cells and molecules leads to progression of inflammation. Clinically, acute inflammation is seen in pneumonia and acute respiratory distress syndrome (ARDS), whereas chronic inflammation is represented by asthma and chronic obstructive pulmonary disease (COPD). Because the lung is a vital organ for gas exchange, excessive inflammation can be life threatening. A delicate balance between inflammation and anti-inflammation is essential for lung homeostasis.

When the lung is exposed to minimal bacterial loads, pathogen clearance operates through innate defenses and the event is generally subclinical. Acute infection results when higher loads of bacteria overcome the local defenses, leading to acute inflammation involving both innate and adaptive defenses. Chronic infection occurs when a marked inflammatory response generated by host defense mechanisms fails to clear the bacteria, with continued tissue destruction.

Viruses activate the innate immune system through cell surface and cytosolic pattern recognition receptors (PRRs), which detect viral components (especially nucleic acids). TLR-3 recognizes double-stranded RNA viruses, while TLR-7 and TLR-8 detect single-stranded RNA viruses. The activated immune cells synthesize antiviral type I interferon (IFN), pro-inflammatory cytokines, and chemokines including TNF-α, IL-β, IL-6, IL-8, IL-12, and monocyte chemoattractant protein.Viruses are also responsible for episodes of exacerbations of COPD and asthma, where they increase the airway inflammation (acute exacerbation or chronic inflammation).

Many protozoa and helminthes involve the respiratory system. Protozoa vary greatly and stimulate distinct immune responses.

The incidence of fungal infections has increased substantially over the past two decades and invasive forms are leading causes of morbidity and mortality, especially amongst immunocompromised or immunosuppressed patients. Disseminated candidiasis, pulmonary aspergillosis, and emerging opportunistic fungi are the most common agents producing these serious mycoses. It is a particular feature of fungi that they are able to generate an extracellular matrix (ECM) that binds them together and allows them to adhere to their in vitro or in vivo substrates. These biofilms serve to protect them against the hostile environments of the host immune system and to resist antimicrobial killing (Kaur and Singh, 2013).

A growing body of research suggests that aspergillus infection may play an important role in clinical asthma (Chishimba et al., 2012; Pasqualotto et al., 2009). Furthermore, recently published work has correlated aspergillus infection with poorer clinical outcomes in patients with COPD (Bafadhel et al., 2013). Similarly cross-sectional studies have shown associations between the presence of Aspergillus spp. and Candida spp. in the sputum and worsened lung function (Chotirmall et al., 2010; Agbetile et al., 2012).

IPF is a chronic and fatal disease primarily characterised by a progressive decline in lung function caused by the scarring of lung tissue which results in worsening dyspnea. Vascular endothelial growth factor (VEGF), fibroblast growth factor (FGF) and platelet derived growth factor (PDGF) are known potent mitogens for fibroblast cells, which then replace the normal tissue in lungs when fibrosis occurs. Lung inflammation has been proposed as a critical factor in IPF (Bringardner et al, 2008). In ILDs, the evidence for a pathogenic role for PDGF, VEGF, and FGF has been demonstrated clinically. The primary site affected is the interstitium, the tissue between the air sacs in the lung, but it does also affect the airspaces, peripheral airways and vessels. The disease process is believed to be initiated by a series of microinjuries to the alveolar epithelium in the lung. After the injury, increased vascular permeability leads to clot formation and resident epithelial cells proliferate in an attempt to replace those cells that died as a result of the injury. This process triggers the release of a variety of growth factors (e.g., PDGF, VEGF, FGF, and transforming growth factor beta (TGF beta), leading to the aberrant activation of the epithelial cells, abnormal vascular remodelling, and most notably, the proliferation and migration of fibroblasts into the lung. Growth factors also induce resident cells to transform into myofibroblasts, which together with fibroblasts organize into foci (King T E Jr, et al., Lancet, 2011, 3; 378(9807):1949-61; Selman M, et al., Ann Intern Med., 2001, 16; 134(2):136-51). These cellular changes result in the disruption of the basement membrane and excessive accumulation of extracellular matrix proteins in the interstitial space. The result is the eventual destruction of the normal architecture of the alveolar capillary unit and lung scarring. The pathologies that define the usual interstitial pattern (UIP) of fibrosis characteristic of IPF are a heterogeneous pattern of alternating areas of normal lung, interstitial inflammation, dense fibrosis, fibroblastic foci, and honeycombing, especially in the subpleural area of the lung (Du Bois R M., Nat Rev Drug Discov., 2010, 9(2):129-40; Selman M, et al., Ann Intern Med., 2001, 16; 134(2):136-51; King T E Jr, et al., Lancet, 2011, 3; 378(9807):1949-61). The loss of normal architecture and scarring of the interstitial space leads to a significant decline in gas exchange capacity leading to development of the classical symptoms of the disease namely dyspnea, chronic cough, inspiratory crackles on auscultation, and abnormal spirometry (Castriotta R J, et al., Chest, 2010, 138(3):693-703). While the disease course is heterogeneous, the median survival is approximately 3-5 years and the most common cause of death is respiratory failure due to the progressive pathologies that disrupt normal lung functioning and gas-exchange.

COPD is a condition in which the underlying inflammation is reported to be substantially resistant to the anti-inflammatory effects of inhaled corticosteroids. Many patients diagnosed with asthma or with COPD continue to suffer from uncontrolled symptoms and from exacerbations of their medical condition that can result in hospitalisation. This occurs despite the use of the most advanced, currently available treatment regimens, comprising of combination products of an inhaled corticosteroid and a long acting β-agonist. Data accumulated over the last decade indicates that a failure to manage effectively the underlying inflammatory component of the disease in the lung is the most likely reason that exacerbations occur. Given the established efficacy of corticosteroids as anti-inflammatory agents and, in particular, of inhaled corticosteroids in the treatment of asthma, these findings have provoked intense investigation. Resulting studies have identified that some environmental insults invoke corticosteroid-insensitive inflammatory changes in patients' lungs. An example is the response arising from virally-mediated upper respiratory tract infections (URTI), which have particular significance in increasing morbidity associated with asthma and COPD.

Background to Phenotypic Drug Discovery

A phenotype is one or more observable features that report changes in the genotype, epigenotype or environmental response of a single cell, a group of cells, organ or of a whole organism. Phenotypic changes are the causes of disease, whether this is a cancer cell undergoing uncontrolled cell division, or a lung cell infected with a coronavirus. For any given disease, there will be an array of phenotypic changes that occur across multiple biological systems. These changes, while complex, mark important distinctions between healthy and diseased states. From a phenotypic perspective, a drug can be considered chemical matter that quantitatively perturbs cellular, organotypic or organismal phenotype(s).

Prior to genetic mapping, the majority of drugs were developed using phenotypic drug discovery (PDD) methods. In PDD, compounds were screened in either in vitro or in vivo models (thought to mimic the disease state of interest) for their ability to modulate phenotypic changes caused by disease. At the time, PDD was highly labour intensive, encouraging the pharmaceutical industry to move towards target-based drug discovery (TDD). For the past three decades, TDD has been the dominant approach to drug discovery in the pharmaceutical industry.

Driven by advances in molecular biology and genomics, TDD defines molecular targets hypothesized to play an important role in disease. TDD has allowed for the efficient screening of large numbers of compounds. However, its reliance on target-based screening has increased the number of failures in clinical trials, often due to poor correlation between novel mechanistic targets and the actual disease state.

Recent technical advances have changed the outlook on PDD methods, as cell-based phenotypic screening and high throughput technologies have enabled scientists to compile immense phenotypic datasets. In recent years, PDD has seen a renaissance and led to discoveries of first-in-class drugs with novel mechanisms of action. The unique promise of PDD is its ability to exploit a disease phenotype to discover novel treatments for diseases for which the root cause is unknown, complex or multifactorial, and for which scientific understanding is insufficient to provide valid molecular targets. PDD can lead to the identification of compounds that act through novel mechanisms. Moreover, compounds identified through PDD methods are more likely to translate to in vivo and clinical efficacy studies than TDD-derived compounds. The recent successes of PDD approaches emphasise the important role played by empirical drug discovery approaches, despite the pharmaceutical industry being dominated by TDD strategies based on molecular target hypotheses.

Phenotypic screening represents a relatively non-biased, disease- and patient-centric approach to drug discovery that embraces biological complexity in order to better reflect the pathophysiology of disease. PDD methods make only limited assumptions about mechanism of action and none about the target, starting instead from a more complex position that is closer to the disease mechanism. In the case of PDD, a ‘physiologically relevant’ biological system or cellular signalling pathway is directly interrogated by chemical matter to identify biologically active compounds. Because PDD embraces biological complexity, the effective chemical matter identified is more likely to translate to success in vivo. PDD can also contribute to improvements over existing therapies by identifying novel physiology for a known target, exploring ‘undrugged’ targets that belong to well known drug target classes or discovering novel MoAs, including new ways of interfering with difficult-to-drug targets.

To function effectively, PDD methods must proceed rationally from disease understanding to a mechanistically defined effect on a pathway or a biomarker to drug, which must then be translated into a therapeutic effect.

The first step in establishing a chain of translatability in PDD is identifying a disease-associated molecular characteristic or signature (for example, a disease-associated gene expression profile) that differentiates the disease state from normal physiology. Having identified the disease characteristics, cellular models aim to reconstruct a cellular phenotype that is as close as possible to the disease condition. For PDD methods to be successful, the assay system must have a clear link to disease (for example, by using patient-derived primary cells) and aim to replicate relevant physiological aspects of the disease. The assay readout should be as proximal as possible to the disease pathophysiology and clinical end point. Sequencing DNA from patients with various diseases and phenotypically profiling them offers an unbiased diagnosis of the similarity between the disease state in humans and the molecular state of the discovery model. It also provides an evaluation of the extent to which a potential therapeutic modifies the molecular state towards the therapeutically desired state. With the appropriate assay, PDD increases both the biological space captured by the assay and the likelihood that the identified compounds and MoA will successfully translate to patients in a clinical setting.

For many disease indications, still we have lack of knowledge about their precise targets and mechanisms. In this situation, TDD approaches favoured by the pharmaceutical industry will be slow, expensive, and have a high failure rate. PDD research offers a promising alternative for finding effective therapeutics before the precise molecular targets are known.

There remains an urgent need to rapidly identify potential therapeutics which may be useful in the treatment or prevention of diseases, such as positive-sense single-stranded RNA virus ((+)ssRNA virus) infection, a disease associated with a positive-sense single-stranded RNA virus ((+)ssRNA virus) infection and lung inflammation.

### BRIEF DESCRIPTION OF THE FIGURES

FIG. 1—heatmap for 23 compounds of the invention (see Example 1). X-axis legend: Cytokines and Inflammatory Response, Activation of C3 and C5, TNF signaling pathway, NOD-like receptor signaling pathway, Rheumatoid arthritis, Chemokine receptors bind chemokines, Cytokine-cytokine receptor interaction, Legionellosis, NF-kappa B signaling pathway, Malaria, interferon alpha/beta signalling, IL23-mediated signalling events, Influenza A, amb2 Intergrin signalling, Selenium Pathway, Peptide ligand-binding receptors, Interferon gamma signaling, Salmonella infection, African trypanosomiasis, Spinal Cord Injury, Measles, Senescence and Autophagy, Hepatitis C, Herpes simplex infection, Validated transcriptional targets of AP1 family mem, Pertussis, NOD1/2 Signaling Pathway, Regulation of Complement cascade, Type II interferon signaling (IFNG), Staphylococcus aureus infection, G alpha (i) signaling events, TWEAK Signaling Pathway, Interferon Signalling, Cytokine Signaling in Immune system, TNF receptor signalling pathway, Hematopoietic cell lineage, Initial triggering of complement, IL27-mediated signaling events, TNF signalling, Calcineurin-regulated NFAT-dependent transcription I, Complement and coagulation cascades, Glycoprotein hormones, Nucleotide-binding domain, leucine rich repeat conta, Jak-STAT signaling pathway, Class A/1 (Rhodopsin-like receptors), CD40/CD40L signaling, Canonical NF-kappaB pathway, Peptide hormone biosynthesis, TGF Beta Signaling Pathway

FIG. 2—heatmap for 50 compounds of the invention (see Example 1). X axis legend: dasatinib, verapamil, NVP-AUY922, lovastatin, fostamatinib, orteronel, PD-184352, minoxidil, navitodax, canertinib, neratinib, trifluoperazine, PD-0325901, curcumin, olaparib, sulforaphane, afatinib, dexamethasone, vorinostat, sirolimus, veliparib, tacrolimus, quinine, lenalidomide, calcitriol, vemurafenib, atorvastatin, EX-527, selumetinib, FK-866, genistein, triptolide, daunorubicin, promazine, artesunate, tivozanib, valproic acid, nimodipine, nicardipine, temozolomide, pelitinib, chloroquine, maraviroc, tozasertib, zileuton, tamoxifen, batimastat, TG-101348, promethazine, teniposide. Y-axis legend IL12_2 PATHWAY, AP1_PATHWAY, SIGNALING_BY_TGF_BETA_FAMILY_MEMBERS, TGF_BETA_SIGNALING_PATHWAY, TERPENOID_BACKBONE_BIOSYNTHESIS, RESPONSE_TO_FLUID_SHEAR_STRESS, RESPONSE_TO_CYTOKINE, IMMUNE_SYSTEM_PROCESS, DEFENSE_RESPONSE, RESPONSE_TO_BIOTIC_STIMULUS, HIF1_TFPATHWAY, VASCULAR_PROCESS_IN_CIRCULATORY_SYSTEM, CERAMIDE_PATHWAY, MAINTENANCE_OF_LOCATION, MATRISOME, REGULATION_OF_INSULIN_LIKE_GROWTH_FACTOR, HEDGEHOG_OFF_STATE, RUNX1_REGULATES_TRANSCRIPTION_OF_GENES_INVOLVED_IN_DIFFERENTIATION_OF_HSCS, SIGNAL_TRANSDUCTION_BY_PROTEIN_PHOSPHORYLATION, MAPK_FAMILY_SIGNALING_CASCADES, THE_ROLE_OF_GTSE1_IN_G2_M_PROGRESSION_AFTER_G2_CHECKPOINT, REGULATION_OF_MRNA_STABILITY_BY_PROTEINS_THAT_BIND_AU_RICH_ELEMENTS, SCF_SKP2_MEDIATED_DEGRADATION_OF_P27_P21, BIOLOGICAL_ADHESION, REG_GR_PATHWAY, KERATINOCYTE_PATHWAY, NATURAL_KILLER_CELL_MEDIATED_CYTOTOXICITY, VIRAL_MYOCARDITIS, CYTOKINE_SIGNALING_IN_IMMUNE_SYSTEM, PATHWAYS_IN_CANCER, DEATH_RECEPTOR_SIGNALLING, DEATH_PATHWAY, HIVNEF_PATHWAY, MAPK_SIGNALING_PATHWAY, EPITHELIAL_CELL_SIGNALING_IN_HELICOBACTER_PYLORI_INFECTION, LOCOMOTION, HOMEOSTATIC_PROCESS, SYMBIOSIS_ENCOMPASSING_MUTUALISM_THROUGH_PARASITISM, RESPONSE_TO_INORGANIC_SUBSTANCE, REPRODUCTION, CIRCULATORY_SYSTEM_DEVELOPMENT, RESPONSE_TO_ENDOGENOUS_STIMULUS, RESPONSE_TO_OXIDATIVE_STRESS, AGING, MOVEMENT_OF_CELL_OR_SUBCELLULAR_COMPONENT, DISEASE, IMMUNE_RESPONSE_REGULATING_CELL_SURFACE_RECEPTOR, INNATE_IMMUNE_SYSTEM, CELL_DEATH, EMBRYO_DEVELOPMENT, RESPONSE_TO_TOPOLOGICALLY_INCORRECT_PROTEIN, CELLULAR_RESPONSE_TO_STRESS, INTRACELLULAR_SIGNAL_TRANSDUCTION, CHEMOKINE_SIGNALING_PATHWAY, EXTRACELLULAR_STRUCTURE_ORGANIZATION, PROTEOLYSIS, CELLULAR_RESPONSE_TO_ORGANIC_SUBSTANCE, DEVELOPMENTAL_BIOLOGY, POST_TRANSLATIONAL_PROTEIN_MODIFICATION, TISSUE_DEVELOPMENT, CELL_ACTIVATION, RESPONSE_TO_ABIOTIC_STIMULUS, RESPONSE_TO_EXTERNAL_STIMULUS, RESPONSE_TO_NITROGEN_COMPOUND, RESPONSE_TO_ORGANIC_CYCLIC_COMPOUND, TXA2PATHWAY, CELL_CELL_SIGNALING, RESPONSE_TO_TOXIC_SUBSTANCE, RESPONSE_TO_LIPID, RESPONSE_TO_OXYGEN_CONTAINING_COMPOUND, HEMOSTASIS, RESPONSE_TO_WOUNDING, CYTOKINE_CYTOKINE_RECEPTOR_INTERACTION, G_PROTEIN_COUPLED_RECEPTOR_SIGNALING_PATHWAY, LYSOPHOSPHOLIPID_PATHWAY, SIGNALING_BY_GPCR, FRA_PATHWAY, REGULATION_OF_RUNX2_EXPRESSION_AND_ACTIVITY, SIGNALING_BY_NOTCH, CELL_CYCLE_ARREST, MAPK_PATHWAY, IL2_1 PATHWAY, JAK_STAT_SIGNALING_PATHWAY, CD40PATHWAYMAP, NOD_LIKE_RECEPTOR_SIGNALING_PATHWAY, CYTOKINE_PRODUCTION, LEISHMANIA_INFECTION, TOLL_LIKE_RECEPTOR_SIGNALING_PATHWAY.

FIG. 3—heatmap for 50 compounds of the invention (see Example 1). X axis legend: mesoridazine, teniposide, tamoxifen, zileuton, promethazine, chloroquine, maraviroc, itraconazole, artesunate, temozolomide, valproic-acid nicardipine, nimodipine, capsaicin, thalidomide, fluspirilene, trifluoperazine, erlotinib, gemcitabine, minoxidil, fostamatinib, lovastatin, promazine, tranylcypromine, cerulenin, daunorubicin, pemetrexed, olaparib, curcumin, tretinoin, mepacrine, carbamazepine, vorinostat, dexamethasone, lapatinib, afatinib, idarubicin, sirolimus forskolin, tolazamide, atorvastatin, vemurafenib, calcitriol, lenalidomide, tacrolimus, quinine, trametinib, verapamil, nitrendipine, dasatinib. Y axis legend: IL12_2 PATHWAY, AP1_PATHWAY, SIGNALING_BY_TGF_BETA_FAMILY_MEMBERS, TGF_BETA_SIGNALING_PATHWAY, TERPENOID_BACKBONE_BIOSYNTHESIS, REGULATION_OF_RUNX2_EXPRESSION_AND_ACTIVITY, REG_GR_PATHWAY, MAPK_PATHWAY, IL2_1 PATHWAY, JAK_STAT_SIGNALING_PATHWAY, CELL_CYCLE_ARREST, SIGNALING_BY_NOTCH, FRA_PATHWAY, MAINTENANCE_OF_LOCATION, RESPONSE_TO_LIPID, RESPONSE_TO_OXYGEN_CONTAINING_COMPOUND, RESPONSE_TO_EXTERNAL_STIMULUS, RESPONSE_TO_NITROGEN_COMPOUND, RESPONSE_TO_ORGANIC_CYCLIC_COMPOUND, HEMOSTASIS, RESPONSE_TO_WOUNDING, CYTOKINE_CYTOKINE_RECEPTOR_INTERACTION, G_PROTEIN_COUPLED_RECEPTOR_SIGNALING_PATHWAY, SIGNALING_BY_GPCR, CELL_CELL_SIGNALING, LYSOPHOSPHOLIPID_PATHWAY, REGULATION_OF_INSULIN_LIKE_GROWTH_FACTOR, RESPONSE_TO_FLUID_SHEAR_STRESS, MATRISOME, AGING, MOVEMENT_OF_CELL_OR_SUBCELLULAR_COMPONENT, RESPONSE_TO_TOXIC_SUBSTANCE, DEATH_RECEPTOR_SIGNALLING, EPITHELIAL_CELL_SIGNALING_IN_HELICOBACTER_PYLORI_INFECTION, KERATINOCYTE_PATHWAY, CYTOKINE_SIGNALING_IN_IMMUNE_SYSTEM, PATHWAYS_IN_CANCER, HOMEOSTATIC_PROCESS, SYMBIOSIS_ENCOMPASSING_MUTUALISM_THROUGH_PARASITISM, LOCOMOTION, REGULATION_OF_MRNA_STABILITY_BY_PROTEINS_THAT_BIND_AU_RICH_ELEMENTS, SCF_SKP2_MEDIATED_DEGRADATION_OF_P27_P21, THE_ROLE_OF_GTSE1_IN_G2_M_PROGRESSION_AFTER_G2_CHECKPOINT, HEDGEHOG_OFF_STATE, RUNX1_REGULATES_TRANSCRIPTION_OF_GENES_INVOLVED_IN_DIFFERENTIATION_OF_HSCS, CELL_DEATH, RESPONSE_TO_ENDOGENOUS_STIMULUS, CIRCULATORY_SYSTEM_DEVELOPMENT, REPRODUCTION, RESPONSE_TO_INORGANIC_SUBSTANCE, RESPONSE_TO_OXIDATIVE_STRESS, IMMUNE_RESPONSE_REGULATING_CELL_SURFACE_RECEPTOR, INNATE_IMMUNE_SYSTEM, DISEASE, SIGNAL_TRANSDUCTION_BY_PROTEIN_PHOSPHORYLATION, MAPK_FAMILY_SIGNALING_CASCADES, PROTEOLYSIS, CELLULAR_RESPONSE_TO_ORGANIC_SUBSTANCE, DEVELOPMENTAL_BIOLOGY, POST_TRANSLATIONAL_PROTEIN_MODIFICATION, TISSUE_DEVELOPMENT, CELLULAR_RESPONSE_TO_STRESS, INTRACELLULAR_SIGNAL_TRANSDUCTION, BIOLOGICAL_ADHESION, MAPK_SIGNALING_PATHWAY, EMBRYO_DEVELOPMENT, RESPONSE_TO_TOPOLOGICALLY_INCORRECT_PROTEIN, TXA2PATHWAY, CERAMIDE_PATHWAY, DEATH_PATHWAY, FMLP_PATHWAY, EXTRACELLULAR_STRUCTURE_ORGANIZATION, VIRAL_MYOCARDITIS, NATURALKILLER_CELL_MEDIATED_CYTOTOXICITY, APOPTOSIS, HIVNEF_PATHWAY, NOD_LIKE_RECEPTOR_SIGNALING_PATHWAY, CYTOKINE_PRODUCTION, LEISHMANIA_INFECTION, TOLL_LIKE_RECEPTOR_SIGNALING_PATHWAY, CD40PATHWAYMAP, DEFENSE_RESPONSE, RESPONSE_TO_CYTOKINE, CELL_ACTIVATION, RESPONSE_TO_ABIOTIC_STIMULUS, IMMUNE_SYSTEM_PROCESS, RESPONSE_TO_BIOTIC_STIMULUS, HIF1_TFPATHWAY, VASCULAR_PROCESS_IN_CIRCULATORY_SYSTEM.

FIGS. 4-26— bar chart plots for individual compounds of the invention (see Example 1)

FIG. 27—combination heatmap for categorisation of 46 compounds of the invention and 4 prior art compounds (see Example 2)

FIG. 28—combination heatmap for categorisation of 45 compounds of the invention and 5 prior art compounds (see Example 2)

FIG. 29— Computed tomography images of the chest as described in Example 5 “RTK inhibitors and inhibitors of RTK pathway”—(A) when requiring mechanical ventilation; (B) two weeks after extubation, requiring highflow nasal cannula oxygen therapy and nasogastric tube feeding; (C) when initiating Compound 134 (nintedanib) therapy; and (D) two months after the implementation of Compound 134 (nintedanib) treatment.

### SUMMARY OF THE INVENTION

The present invention provides a compound selected from Compounds 1 to 203 (as defined in Table 1) and prodrugs thereof and pharmaceutically acceptable salts and solvates thereof (“compounds of the invention”) for use in the treatment or prevention of a disease selected from the group consisting of a positive-sense single-stranded RNA virus ((+)ssRNA virus) infection, a disease associated with a positive-sense single-stranded RNA virus ((+)ssRNA virus) infection and lung inflammation.

### DETAILED DESCRIPTION OF THE INVENTION

The present invention also provides a method of treatment or prevention of a disease selected from the group consisting of a positive-sense single-stranded RNA virus ((+)ssRNA virus) infection, a disease associated with a positive-sense single-stranded RNA virus ((+)ssRNA virus) infection and lung inflammation in a subject by administering to said subject an effective amount of a compound selected from Compounds 1 to 203 and prodrugs thereof and pharmaceutically acceptable salts and solvates thereof.

The present invention also provides use of a compound selected from Compounds 1 to 203 and prodrugs thereof and pharmaceutically acceptable salts and solvates thereof in the manufacture of a medicament for the treatment or prevention of a disease selected from the group consisting of a positive-sense single-stranded RNA virus ((+)ssRNA virus) infection, a disease associated with a positive-sense single-stranded RNA virus ((+)ssRNA virus) infection and lung inflammation.

The compound selected from Compounds 1 to 203 may optionally be utilised in the form of a pharmaceutically acceptable salt and/or solvate and/or prodrug.

Compounds

In one embodiment, the compound of the invention is selected from the group consisting of Compounds 1 to 203 as defined in Table 1.

TABLE 1
Compounds of the invention

| Comp. | |
|---|---|
| No | Compound Name |
| 1 | pravastatin |
| 2 | 17-hydroxyprogesterone-caproate |
| 3 | acrivastatin |
| 4 | alprenolol |
| 5 | amiloride |
| 6 | amitriptyline |
| 7 | amoxapine |
| 8 | atorvastatin |
| 9 | atovaquone |
| 10 | betamethasone |
| 11 | bezafibrate |
| 12 | budipine |
| 13 | calcipotriol |
| 14 | capsaicin |
| 15 | carbenoxolone |
| 16 | chlorphenamine |
| 17 | ciclosporin |
| 18 | clomethiazole |
| 19 | clomifene |
| 20 | clozapine |
| 21 | curcumin |
| 22 | cyclosporin-a |
| 23 | diazepam |
| 24 | enoxacin |
| 25 | estradiol |
| 26 | esmolol |
| 27 | etofylline clofibrate |
| 28 | estriol |
| 29 | flunisolide |
| 30 | felodipine |
| 31 | flecainide |
| 32 | hydrocortisone |
| 33 | fluphenazine |
| 34 | fluvoxamine |
| 35 | fostamatinib |
| 36 | linezolid |
| 37 | isoxsuprine |
| 38 | labetalol |
| 39 | lenalidomide |
| 40 | mazindol |
| 41 | lobeline |
| 42 | loratadine |
| 43 | lovastatin |
| 44 | maprotiline |
| 45 | methoxsalen |
| 46 | mepyramine |
| 47 | naphazoline |
| 48 | neostigmine |
| 49 | pimavanserin |
| 50 | nicergoline |
| 51 | nifedipine |
| 52 | nitrendipine |
| 53 | norethindrone |
| 54 | paclitaxel |
| 55 | palonosetron |
| 56 | raloxifene |
| 57 | pindolol |
| 58 | prednisone |
| 59 | proguanil |
| 60 | promazine |
| 61 | saxagliptin |
| 62 | repaglinide |
| 63 | rivaroxaban |
| 64 | rosuvastatin |
| 65 | salmeterol |
| 66 | sulpiride |
| 67 | scopolamine |
| 68 | simvastatin |
| 69 | ticagrelor |
| 70 | tamoxifen |
| 71 | valsartan |
| 72 | triamcinolone |
| 73 | trifluoperazine |
| 74 | trimethobenzamide |
| 75 | trimipramine |
| 76 | ubenimex |
| 77 | valproic-acid |
| 78 | zonisamide |
| 79 | ibudilast |
| 80 | desloratadine |
| 81 | cefoxitin |
| 82 | ornidazole |
| 83 | erythromycin |
| 84 | mitotane |
| 85 | pazopanib |
| 86 | temozolomide |
| 87 | trametinib |
| 88 | cilnidipine |
| 89 | dilazep |
| 90 | naftidrofuryl |
| 91 | fulvestrant |
| 92 | mestranol |
| 93 | bucladesine |
| 94 | spiperone |
| 95 | metergoline |
| 96 | niacin |
| 97 | dihydroergocristine |
| 98 | ondansetron |
| 99 | tolterodine |
| 100 | rho-kinase inhibitor |
| 101 | thioridazine |
| 102 | 616-dimethylprostag |
| 103 | 6-aminochrysene |
| 104 | cp72-4714 |
| 105 | gossypol |
| 106 | trichostatin-a |
| 107 | tozasertib |
| 108 | dppe |
| 109 | ib-meca |
| 110 | indole-3-carbinol |
| 111 | remacemide |
| 112 | saracatinib |
| 113 | dasatinib |
| 114 | cerulenin |
| 115 | batimastat |
| 116 | nvp-auy922 |
| 117 | canertinib |
| 118 | afatinib |
| 119 | vorinostat |
| 120 | erlotinib |
| 121 | 3-amino-benzamide |
| 122 | veliparib |
| 123 | linsitinib |
| 124 | carbamazepine |
| 125 | px-12 |
| 126 | idarubicin |
| 127 | foretinib |
| 128 | mk-1775 |
| 129 | tipifarnib |
| 130 | elesclomol |
| 131 | gdc-0941 |
| 132 | flutamide |
| 133 | ibrutinib |
| 134 | nintedanib |
| 135 | rilmenidine |
| 136 | temsirolimus |
| 137 | etoposide |
| 138 | tivantinib |
| 139 | sulforaphane |
| 140 | selumetinib |
| 141 | tivozanib |
| 142 | calcitriol |
| 143 | vemurafenib |
| 144 | promethazine |
| 145 | artesunate |
| 146 | minoxidil |
| 147 | fk-866 |
| 148 | triptolide |
| 149 | daunorubicin |
| 150 | genistein |
| 151 | tg-101348 |
| 152 | mepacrine |
| 153 | pemetrexed |
| 154 | forskolin |
| 155 | gemcitabine |
| 156 | alvocidib |
| 157 | tretinoin |
| 158 | fluspirilene |
| 159 | orantinib |
| 160 | tanespimycin |
| 161 | buparlisib |
| 162 | sn-38 |
| 163 | bortezomib |
| 164 | equol |
| 165 | clonidine |
| 166 | navitoclax |
| 167 | quinine |
| 168 | zileuton |
| 169 | verapamil |
| 170 | maraviroc |
| 171 | teniposide |
| 172 | rucaparib |
| 173 | mk-2206 |
| 174 | itraconazole |
| 175 | neratinib |
| 176 | pd-0325901 |
| 177 | olaparib |
| 178 | orteronel |
| 179 | nicardipine |
| 180 | nimodipine |
| 181 | pelitinib |
| 182 | pd-184352 |
| 183 | dalcetrapib |
| 184 | mesoridazine |
| 185 | tolazamide |
| 186 | etomoxir |
| 187 | lapatinib |
| 188 | rolipram |
| 189 | bi-2536 |
| 190 | enzalutamide |
| 191 | bms-754807 |
| 192 | resveratrol |
| 193 | tak-715 |
| 194 | nilotinib |
| 195 | losartan |
| 196 | estrone |
| 197 | imiquimod |
| 198 | enmd-2076 |
| 199 | cp-724714 |
| 200 | decitabine |
| 201 | ex-527 |
| 202 | phenethyl-isothiocyanate |
| 203 | tranylcypromine |
| — | — |

PD-0325901 is the code name for a compound of the following formula:

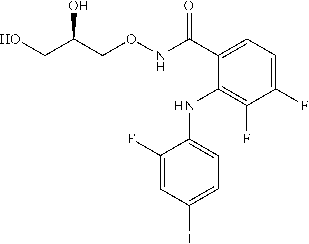 [SMILES: O=C(NOC[C@H](O)CO)c1ccc(F)c(F)c1Nc1ccc(I)cc1F]

NVP-AUY922 is the code name for a compound of the following formula:

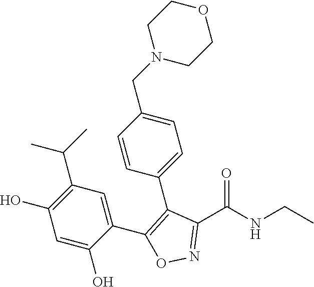 [SMILES: CCNC(=O)c1noc(-c2cc(C(C)C)c(O)cc2O)c1-c1ccc(CN2CCOCC2)cc1]

PD-184352 is the code name for a compound of the following formula:

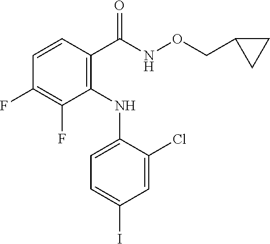 [SMILES: O=C(NOCC1CC1)c1ccc(F)c(F)c1Nc1ccc(I)cc1Cl]

FK-866 is also known as daporinad.

TG-101348 is also known as fedratinib.

CP-72-4714 is the code name for a compound of the following formula:

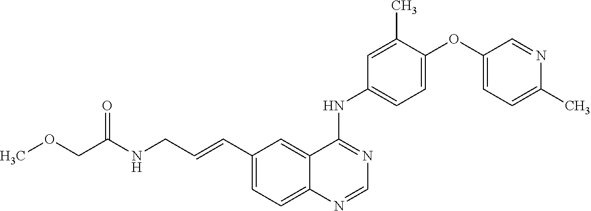

PX-12 is the code name for a compound of the following formula:

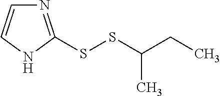

MK-1775 is also known as Adavosertib.

GDC-0941 is also known as Pictilisib.

MK-2206 is the code name for a compound of the following formula:

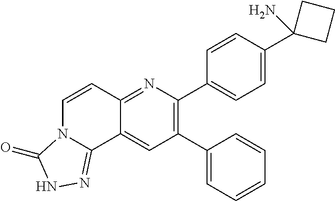 [SMILES: NC1(c2ccc(-c3nc4ccn5c(=O)[nH]nc5c4cc3-c3ccccc3)cc2)CCC1]

BI-2536 is the code name for a compound of the following formula:

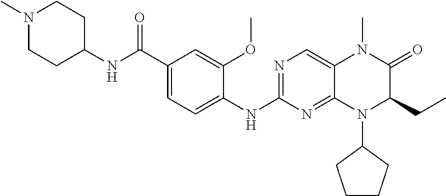 [SMILES: CC[C@@H]1C(=O)N(C)c2cnc(Nc3ccc(C(=O)NC4CCN(C)CC4)cc3OC)nc2N1C1CCCC1]

BMS-754807 is the code name for a compound of the following formula:

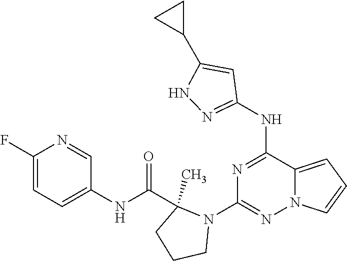 [SMILES: C[C@@]1(C(=O)Nc2ccc(F)nc2)CCCN1c1nc(Nc2cc(C3CC3)[nH]n2)c2cccn2n1]

TAK-715 is the code name for a compound of the following formula:

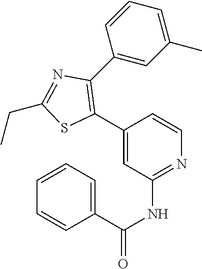 [SMILES: CCc1nc(-c2cccc(C)c2)c(-c2ccnc(NC(=O)c3ccccc3)c2)s1]

ENMD-2076 is the code name for a compound of the following formula:

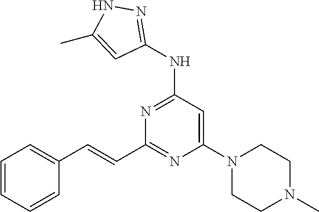 [SMILES: Cc1cc(Nc2cc(N3CCN(C)CC3)nc(/C=C/c3ccccc3)n2)n[nH]1]

CP-724714 is the code name for a compound of the following formula:

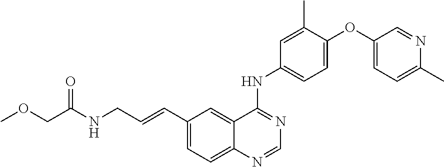

EX-527 is the code name for a compound of the following formula:

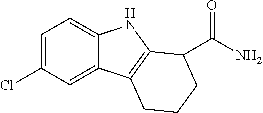 [SMILES: NC(=O)C1CCCc2c1[nH]c1ccc(Cl)cc21]

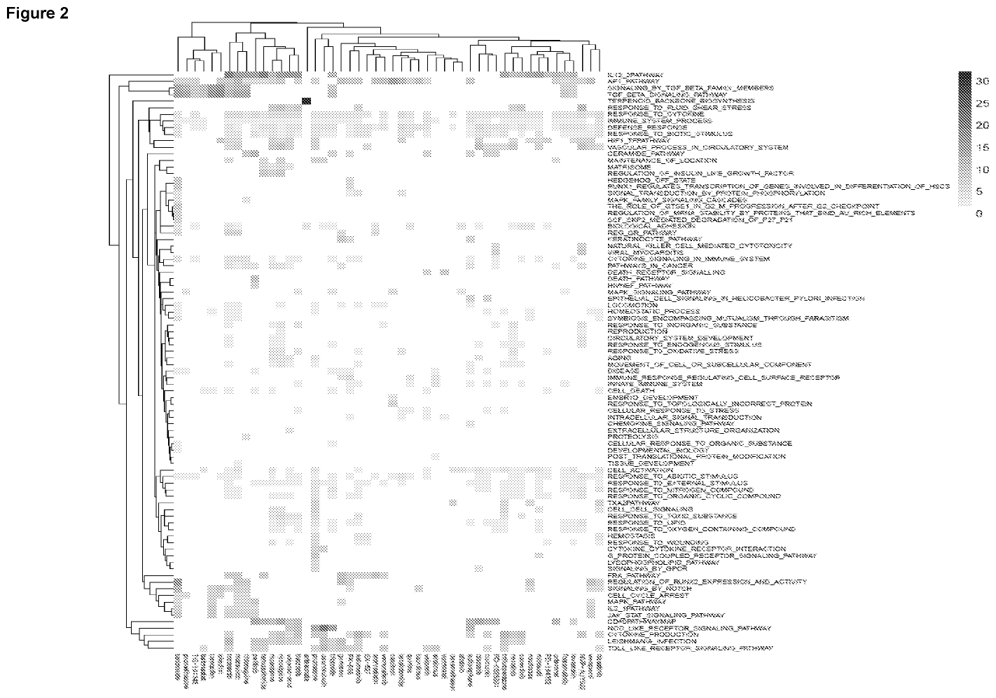

Suitable compounds of the invention are listed in Table 2 (see also FIG. 2).

TABLE 2
Suitable compounds of the invention

| | |
|---|---|
| PD-0325901 | zileuton |
| sulforaphane | lenalidomide |
| neratinib | promethazine |
| curcumin | canertinib |
| trifluoperazine | afatinib |
| olaparib | vorinostat |
| valproic-acid | PD-184352 |
| fostamatinib | triptolide |
| selumetinib | temozolomide |
| tivozanib | daunorubicin |
| tozasertib | genistein |
| calcitriol | verapamil |
| lovastatin | artesunate |
| tamoxifen | minoxidil |
| NVP-AUY922 | FK-866 |
| vemurafenib | erlotinib |
| nicardipine | dasatinib |
| promazine | veliparib |
| orteronel | TG-101348 |
| atorvastatin | mepacrine |
| nimodipine | maraviroc |
| batimastat | teniposide |
| navitoclax | 3-amino-benzamide |
| EX-527 | pemetrexed |
| quinine | mesoridazine |
| pelitinib | dalcetrapib |

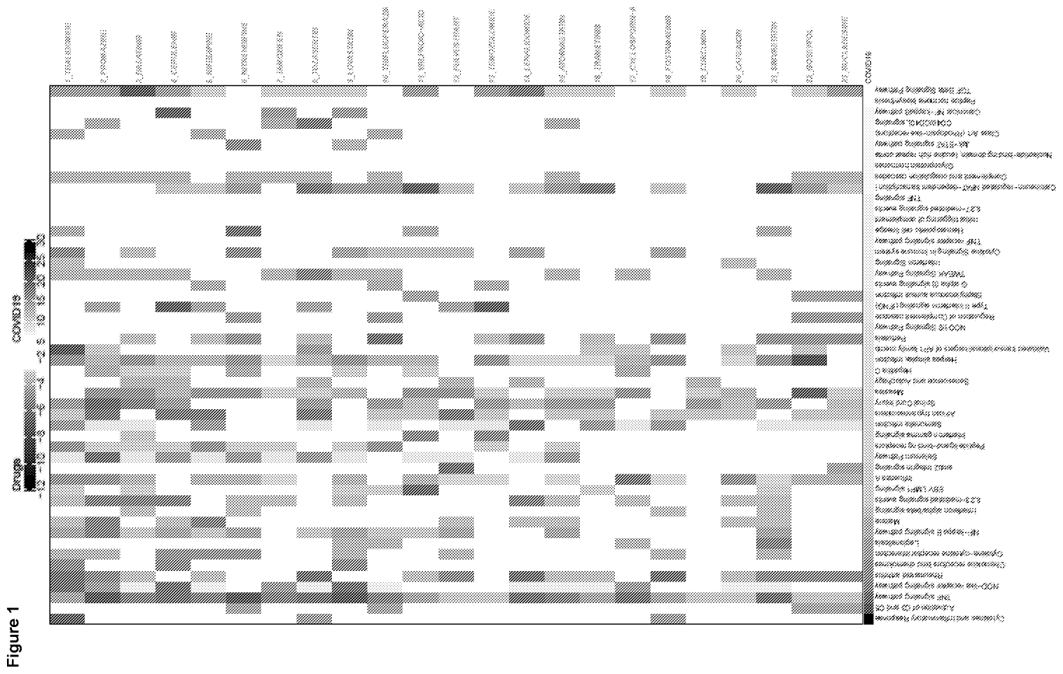

Other suitable compounds of the invention are listed in Table 3 (see also FIG. 1).

TABLE 3
Other suitable compounds of the invention.

| | |
|---|---|
| atorvastatin | trifluoperazine |
| capsaicin | valproic-acid |
| curcumin | temozolomide |
| cyclosporin-a | trametinib |
| fostamatinib | fulvestrant |
| lenalidomide | bucladesine |
| lovastatin | gossypol |
| nifedipine | tozasertib |
| nitrendipine | dasatinib |
| promazine | cerulenin |
| simvastatin | — |
| tamoxifen | — |

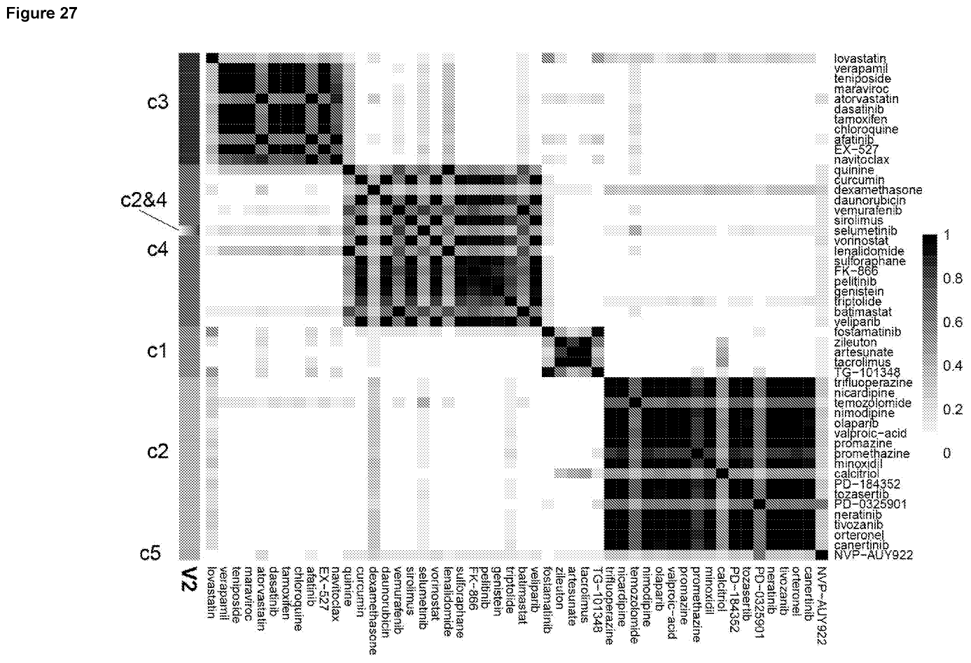

The invention also provides further suitable compounds of the invention listed Table 4 (see also FIG. 27).

TABLE 4
Further suitable compounds of the invention

| | |
|---|---|
| neratinib | EX-527 |
| sulforaphane | NVP-AUY922 |
| PD-0325901 | pelitinib |
| olaparib | lenalidomide |
| trifluoperazine | zileuton |
| valproic-acid | promethazine |
| curcumin | PD-184352 |
| fostamatinib | triptolide |
| selumetinib | minoxidil |
| lovastatin | genistein |
| tamoxifen | verapamil |
| tozasertib | canertinib |
| tivozanib | afatinib |
| calcitriol | vorinostat |
| nicardipine | temozolomide |
| batimastat | FK-866 |
| orteronel | daunorubicin |
| atorvastatin | artesunate |
| nimodipine | TG-101348 |
| navitoclax | maraviroc |
| promazine | veliparib |
| quinine | teniposide |
| vemurafenib | dasatinib |

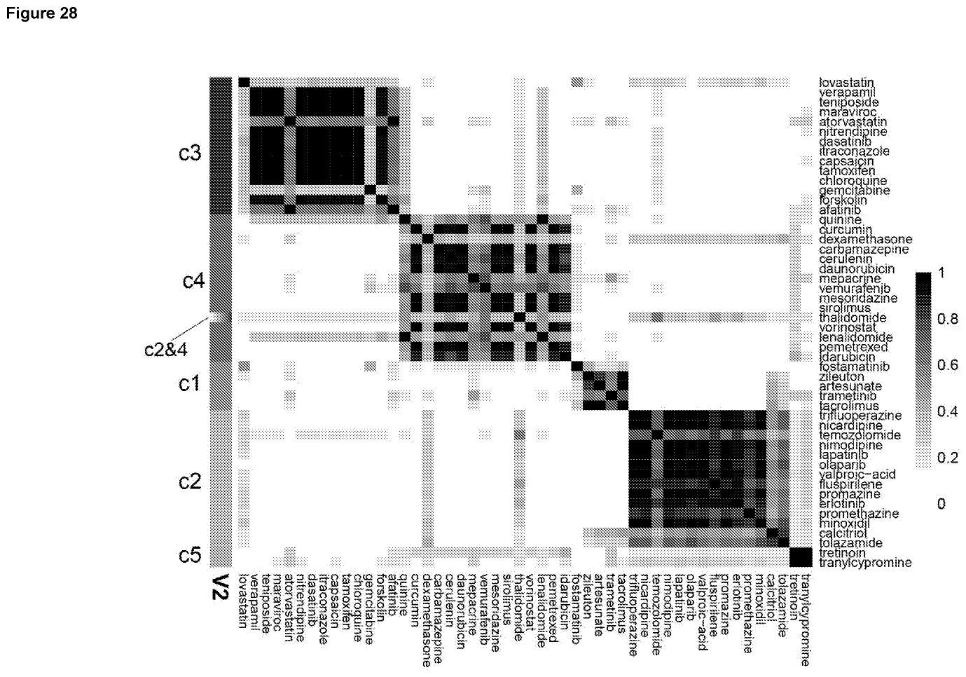

The invention also provides additional suitable compounds listed in Table 5 (see FIG. 28). The compounds of Table 5 are expected to be particularly useful in the treatment or prevention of disease, such as a positive-sense single-stranded RNA virus ((+)ssRNA virus) infection, for example a coronavirus, in particular SARS-CoV-2. The compounds of Table 5 are also expected to be particularly useful in the treatment or prevent of a disease associated with a positive-sense single-stranded RNA virus ((+)ssRNA virus) infection, in particular COVID-19.

TABLE 5
Additional suitable compounds of the invention

| | |
|---|---|
| olaparib | daunorubicin |
| trifluoperazine | artesunate |
| valproic-acid | maraviroc |
| curcumin | teniposide |
| fostamatinib | dasatinib |
| lovastatin | erlotinib |
| tamoxifen | pemetrexed |
| calcitriol | mesoridazine |
| nicardipine | mepacrine |
| atorvastatin | tolazamide |
| nimodipine | cerulenin |
| promazine | trametinib |
| quinine | tretinoin |
| vemurafenib | idarubicin |
| lenalidomide | gemcitabine |
| zileuton | forskolin |
| promethazine | capsaicin |
| minoxidil | lapatinib |
| verapamil | carbamazepine |
| afatinib | itraconazole |
| vorinostat | nitrendipine |
| temozolomide | fluspirilene |
| — | tranylcypromine |

In one embodiment, the compound of the invention is selected from the group consisting of PD-0325901, sulforaphane, neratinib, curcumin, trifluoperazine, olaparib, valproic-acid, fostamatinib, selumetinib, tivozanib, tozasertib, calcitriol, lovastatin, tamoxifen, NVP-AUY922, vemurafenib, nicardipine, promazine, orteronel and atorvastatin.

Suitably, the compound of the invention is selected from the group consisting of PD-0325901, sulforaphane, neratinib, curcumin, trifluoperazine, olaparib, valproic-acid, fostamatinib, selumetinib and tivozanib.

Suitably, the compound of the invention is selected from the group consisting of PD-0325901, sulforaphane, neratinib, curcumin and trifluoperazine.

Suitably, the compound of the invention is selected from the group consisting of PD-0325901, sulforaphane and neratinib. In one embodiment of the invention, the compound selected from Compounds 1 to 203 is PD-0325901. In one embodiment of the invention the compound selected from Compounds 1 to 203 is sulforaphane. In one embodiment of the invention, the compound selected from Compounds 1 to 203 is neratinib.

Suitably, the compound of the invention is selected from the group consisting of betamethasone, calcitriol, curcumin, desloratadine, felodipine, fostamatinib, loratadine, lovastatin, nifedipine, pravastatin, promethazine, resveratrol, rosuvastatin and triamcinolone.

In one embodiment, the compound of the invention has an anti-viral effect. In one embodiment, the compound of the invention has a beneficial effect on the host system and in particular has an anti-inflammatory effect.

Certain compounds of the invention may have an immune-stimulatory effect and would be suitable for use as a treatment in the early phase of infection by a positive-sense single-stranded RNA virus (or other pathogen). In the later phase of infection, an anti-inflammatory (immune-suppressive) effect of the treatment compound would be more suitable.

Therapeutic Methods

The term “treatment” means the alleviation of disease or symptoms of disease. The term “prevention” means the prevention of disease or symptoms of disease. Treatment includes treatment alone or in conjunction with other therapies. Treatment embraces treatment leading to improvement of the disease or its symptoms or slowing of the rate of progression of the disease or its symptoms. Treatment includes prevention of relapse.

The term “effective amount” refers to an amount effective, at dosages and for periods of time necessary, to achieve the desired therapeutic result, in which any toxic or detrimental effects of the pharmacological agent are outweighed by the therapeutically beneficial effects. It is understood that the effective dosage will be dependent upon the age, sex, health, and weight of the recipient, kind of concurrent treatment, if any, frequency of treatment, and the nature of the effect desired. The most preferred dosage will be tailored to the individual subject, as is understood and determinable by one of skill in the art, without undue experimentation. Example dosages are discussed below.

As used herein, a “subject” is any mammal, including but not limited to humans, non-human primates, farm animals such as cattle, sheep, pigs, goats and horses; domestic animals such as cats, dogs, rabbits; laboratory animals such as mice, rats and guinea pigs that exhibit at least one symptom associated with a disease, have been diagnosed with a disease, or are at risk for developing a disease. The term does not denote a particular age or sex. Suitably the subject is a human subject.

The term “coronavirus” includes members of the subfamily Coronavirinae in the family Coronaviridae.

Diseases or disorders that may be mediated by treatment with a compound of the invention may be selected from the list below.

In one embodiment the disease is selected from the group consisting of a positive-sense single-stranded RNA virus ((+)ssRNA virus) infection, a disease associated with a positive-sense single-stranded RNA virus ((+)ssRNA virus) infection and lung inflammation.

Positive-Sense Single-Stranded RNA Virus Infection

A positive-sense single-stranded RNA virus (or (+)ssRNA virus) is a virus that uses positive sense single stranded RNA as its genetic material. Therefore, in one embodiment the disease is a positive-sense single-stranded RNA virus ((+)ssRNA virus) infection. Suitably, the (+)ssRNA virus is a picornavirus, an astrovirus, a calicivirus, a hepevirus, a flavivirus, a togavirus, an arterivirus or a coronavirus, especially a coronavirus.

In one embodiment, the (+)ssRNA virus is a coronavirus. Suitably, the coronavirus is an Alphacoronavirus, a Betacoronavirus, a Gammacoronavirus and a Deltacoronavirus, especially a Betacoronavirus. More suitably, the coronavirus is selected from the group consisting of SARS-CoV, SARS-CoV-2, MERS-CoV, HCoV-NL63, HCoV-229E, HCov-OC43 and HKU1. Even more suitably, the coronavirus is SARS-CoV. Even more suitably, the coronavirus is SARS-CoV-2. Even more suitably, the coronavirus is MERS-CoV.

Disease Associated with Positive-Sense Single-Stranded RNA Virus Infection

Positive-sense RNA viruses account for a large fraction of known viruses, including many pathogens such as the hepacivirus C, West Nile virus, dengue virus, SARS and MERS coronaviruses, and SARS-CoV-2 as well as less clinically serious pathogens such as the rhinoviruses that cause the common cold. Therefore, in one embodiment the disease is a disease associated with a positive-sense single-stranded RNA virus ((+)ssRNA virus) infection. Suitably the disease is selected from the group consisting of SARS, MERS and COVID-19. More suitably, the disease is SARS. More suitably, the disease is MERS. More suitably, the disease is COVID-19.

Diseases associated with positive-sense single-stranded RNA virus infection include various complications arising therefrom. Complications include respiratory distress, pulmonary fibrosis, pneumonia, cytokine storm; acute liver injury, septic shock, acute kidney injury, pancreatic injury, peripheral nervous systems complications (such as an impaired ability to taste, to smell, and vision impairment), muscle pain, inflammation of cardiac muscle, blood clots in veins, decreased blood flow in coronary arteries, cardiogenic shock, heart failure, impaired consciousness, brain inflammation, irritation and swelling of brain and blood vessels, acute cerebrovascular complications (such as stroke, seizures and slurred speech), arrhythmia, myocarditis, thrombotic events rhabdomyolysis, neurocognitive deficits, and sensory and motor deficits. The present invention embraces the complications caused by SARS-CoV-2 infection.

Lung Inflammation

In one embodiment the disease is lung inflammation. Suitably, the lung inflammation is caused by pathogenic infection, bacterial infection, fungal infection or viral infection, in particular a (+)ssRNA virus infection. More suitably, the lung inflammation is caused by a disease selected from the group consisting of pneumonia, acute respiratory disease symptom (ARDS), COPD, asthma, idiopathic pulmonary fibrosis, allergic rhinitis, rhinitis and sinusitis. More suitably, the lung inflammation is caused by COPD, asthma or idiopathic pulmonary fibrosis. Even more suitably, the lung inflammation is caused by COPD. Even more suitably, the lung inflammation is caused by asthma. Even more suitably, the lung inflammation is caused by idiopathic pulmonary fibrosis.

In one embodiment, the compounds of the invention are used for the treatment of hyperinflammation associated with positive-sense single-stranded RNA virus infection, such as coronavirus infection e.g. the compounds of the invention reduce hyperinflammation associated with coronavirus infection.

Forms of Compounds and Pharmaceutical Compositions

In one embodiment of the invention the compounds of the invention are utilised in the form of a pharmaceutically acceptable salt.

It will be appreciated that for use in medicine the salts of the compound selected from Compounds 1 to 203 should preferably be pharmaceutically acceptable. Suitable pharmaceutically acceptable salts will be apparent to those skilled in the art. Pharmaceutically acceptable salts include those described by Berge, Bighley and Monkhouse J. Pharm. Sci. (1977) 66, pp 1-19. Such pharmaceutically acceptable salts include acid addition salts formed with inorganic acids e.g. hydrochloric, hydrobromic, sulphuric, nitric or phosphoric acid and organic acids e.g. succinic, maleic, acetic, fumaric, citric, tartaric, benzoic, p-toluenesulfonic, methanesulfonic or naphthalenesulfonic acid. Other salts e.g. oxalates or formates, may be used, for example in the isolation of compounds of formula (I) and are included within the scope of this invention, as are basic addition salts such as sodium, potassium, calcium, aluminium, zinc, magnesium and other metal salts. Pharmaceutically acceptable salts may also be formed with organic bases e.g. with ammonia, meglumine, tromethamine, piperazine, arginine, choline, diethylamine, benzathine or lysine.

A compound selected from Compounds 1 to 203 and prodrugs thereof and pharmaceutically acceptable salts and solvates thereof may be prepared in crystalline or non-crystalline form and, if crystalline, may optionally be solvated, e.g. as the hydrate. This invention includes within its scope stoichiometric solvates (e.g. hydrates) as well as compounds containing variable amounts of solvent (e.g. water). Thus, in a further embodiment of the invention the compounds of the invention are utilised in the form of a pharmaceutically acceptable solvate.

In a further embodiment of the invention the compounds of the invention are not in the form of a salt or solvate.

It will be understood that any compound of the invention may be used in the form of a prodrug or a pharmaceutically acceptable salt or solvate thereof.

A “prodrug” is a compound which upon administration to the recipient is capable of providing (directly or indirectly) the compound selected from Compounds 1 to 203 or an active metabolite or residue thereof.

In an embodiment of the invention the compounds of the invention are utilised in the form of a prodrug. In a further embodiment of the invention the compounds of the invention are not utilised in the form of a prodrug.

Isotopically-labelled compounds which are identical to Compounds 1 to 203 but for the fact that one or more atoms are replaced by an atom having an atomic mass or mass number different from the atomic mass or mass number most commonly found in nature, or in which the proportion of an atom having an atomic mass or mass number found less commonly in nature has been increased (the latter concept being referred to as “isotopic enrichment”) are also contemplated for the uses and method of the invention. Examples of isotopes that can be incorporated into Compounds 1 to 203 include isotopes of hydrogen, carbon, nitrogen, oxygen, fluorine, iodine and chlorine such as ²H (deuterium), ³H, ¹¹C, ¹³C, ¹⁴C, ¹⁸F, ¹²³I or ¹²⁵I, which may be naturally occurring or non-naturally occurring isotopes.

Compounds 1 to 203 and pharmaceutically acceptable salts of Compounds 1 to 203 that contain the aforementioned isotopes and/or other isotopes of other atoms are contemplated for use for the uses and method of the present invention. Isotopically labelled Compounds 1 to 203, for example Compounds 1 to 203 into which radioactive isotopes such as ³H or ¹⁴C have been incorporated, are useful in drug and/or substrate tissue distribution assays. Tritiated, i.e. ³H, and carbon-14, i.e. ¹⁴C, isotopes are particularly preferred for their ease of preparation and detectability. ¹¹C and ¹⁸F isotopes are particularly useful in PET (positron emission tomography).

Since Compounds 1 to 203 are intended for use in pharmaceutical compositions it will readily be understood that it is preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

Administration

For use in therapy the compounds of the invention are usually administered as a pharmaceutical composition. The invention also provides a pharmaceutical composition comprising a compound selected from Compounds 1 to 203 or a prodrug thereof and a pharmaceutically acceptable salt thereof and a solvate thereof, and a pharmaceutically acceptable carrier.

The compounds of the invention may be administered by any convenient method, e.g. by oral, parenteral, buccal, sublingual, nasal, rectal, intrathecal or transdermal administration or by inhalation (e.g. for topical administration to the lung by inhalation), and the pharmaceutical compositions adapted accordingly.

A compound of the invention which is active when given orally can be formulated as liquids or solids, e.g. as syrups, suspensions, emulsions, tablets, capsules or lozenges.

A liquid formulation will generally consist of a suspension or solution of the active ingredient in a suitable liquid carrier(s) e.g. an aqueous solvent such as water, ethanol or glycerine, or a non-aqueous solvent, such as polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatin capsule; alternatively a dispersion or suspension can be prepared using any suitable pharmaceutical carrier(s), e.g. aqueous gums, celluloses, silicates or oils and the dispersion or suspension then filled into a soft gelatin capsule.

Typical parenteral compositions consist of a solution or suspension of the active ingredient in a sterile aqueous carrier or parenterally acceptable oil, e.g. polyethylene glycol, polyvinyl pyrrolidone, lecithin, arachis oil or sesame oil. Alternatively, the solution can be optimized and then reconstituted with a suitable solvent just prior to administration.

Compositions for nasal administration or for inhalation (e.g. for topical administration to the lung by inhalation) may conveniently be formulated as aerosols, drops, gels and powders. Aerosol formulations typically comprise a solution or fine suspension of the active ingredient in a pharmaceutically acceptable aqueous or non-aqueous solvent and are usually presented in single or multidose quantities in sterile form in a sealed container which can take the form of a cartridge or refill for use with an atomising device. Alternatively the sealed container may be a disposable dispensing device such as a single dose nasal inhaler or an aerosol dispenser fitted with a metering valve suitably for delivery of the aerosol to the nasal or bronchial passages. Where the dosage form comprises an aerosol dispenser, it will contain a propellant which can be a compressed gas e.g. air, or an organic propellant such as a fluorochlorohydrocarbon or hydrofluorocarbon. Aerosol dosage forms can also take the form of pump-atomisers.

Topical administration to the lung may also be achieved by use of a dry-powder formulation which contains the compound of the invention in finely divided form optionally together with one or more carriers or other excipients. A dry powder formulation is typically delivered using a dry powder inhaler (DPI) device. Example dry powder delivery systems include TURBOHALER®, DISKUS® and ELLI PTA®.

Compositions suitable for buccal or sublingual administration include tablets, lozenges and pastilles where the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches. In one embodiment the composition is in unit dose form such as a tablet, capsule or ampoule.

The composition may contain from 0.1% to 100% by weight, for example from 10 to 60% by weight, of the active material, depending on the method of administration. The composition may contain from 0% to 99% by weight, for example 40% to 90% by weight, of the carrier, depending on the method of administration. The composition may contain from 0.05 mg to 1000 mg, for example from 1.0 mg to 500 mg, of the active material, depending on the method of administration. The composition may contain from 50 mg to 1000 mg, for example from 100 mg to 400 mg of the carrier, depending on the method of administration. The dose of the compound used in the treatment of the aforementioned disorders will vary in the usual way with the seriousness of the disorders, the weight of the sufferer, and other similar factors. However, as a general guide suitable unit doses may be 0.01 to 1000 mg, more suitably 1.0 to 500 mg especially for oral administration. In general, smaller unit doses may be appropriate for topical administration to the nose or lung e.g. 0.01 to 1 mg. Such unit doses may be administered more than once a day, for example two or three a day. Such therapy may extend for a number of weeks or months.

Combinations

In one embodiment of the invention, the compound selected from Compounds 1 to 203 is used in combination with a further therapeutic agent or agents. When the compounds selected from Compounds 1 to 203 are used in combination with other therapeutic agents, the compounds may be administered either sequentially or simultaneously by any convenient route.

Alternatively, the compounds may be administered separately.

In one embodiment of the invention, the compound selected from Compounds 1 to 203 is used in combination with a second or further compound selected from Compounds 1 to 203. Suitably, at least compound selected from Compounds 1 to 203 has an anti-viral effect. Suitably, at least one compound selected from Compounds 1 to 203 has a beneficial effect on the host system and in particular has an anti-inflammatory effect.

In one embodiment of the invention, the compound selected from Compounds 1 to 203 is used in combination with a second compound selected from Compounds 1 to 203. Suitably, both compounds selected from Compounds 1 to 203 have anti-viral effect. Suitably, both compounds selected from Compounds 1 to 203 have a beneficial effect on the host system and in particular have anti-inflammatory effect. Suitably, one compound selected from Compounds 1 to 203 has an anti-viral effect and the other have a beneficial effect on the host system and in particular has an anti-inflammatory effect.

Suitably by reference to the categories C1, C2, C3, C4 and C5 below a compound from one category is used in combination with a compound from a different category (see FIG. 27).

However for a stronger perturbation of the same disease pathways compounds from the same category may be combined (see Example 2).

Category C1

Fostamatinib, zileuton, artesunate, tacrolimus* and TG-101348.

Category C2

Neratinib, PD-0325901, olaparib, trifluoperazine, valproic-acid, tozasertib, tivozanib, calcitriol, nicardipine, orteronel, nimodipine, promazine, promethazine, PD-184352, minoxidil, canertinib, temozolomide and selumetinib.

Category C3

Lovastatin, tamoxifen, atorvastatin, navitoclax, EX-527, chloroquine*, verapamil, afatinib, maraviroc, teniposide and dasatinib.

Category C4

Sulforaphane, curcumin, dexamethasone*, sirolimus*, batimastat, quinine, vemurafenib, pelitinib, lenalidomide, triptolide, genistein, vorinostat, FK-866, daunorubicin, veliparib and selumetinib.

Category C5

NVP-AUY922.

Suitably by reference to the categories CC1, CC2, CC3, CC4 and CC5 below a compound from one category is used in combination with a compound from a different category (see FIG. 28). However for a stronger perturbation of the same disease pathways compounds from the same category may be combined (see Example 2).

Category CC1

Fostamatinib, zileuton, artesunate, tacrolimus* and trametinib

Category CC2

Olaparib, trifluoperazine, valproic-acid, calcitriol, nicardipine, nimodipine, promazine, promethazine, minoxidil, temozolomide, erlotinib, tolazamide, lapatinib, fluspirilene and thalidomide*

Category CC3

Lovastatin, tamoxifen, atorvastatin, chloroquine*, verapamil, afatinib, maraviroc, teniposide, dasatinib, gemcitabine, forskolin, capsaicin, itraconazole and nitrendipine

Category CC4

Curcumin, dexamethasone*, sirolimus*, quinine, vemurafenib, lenalidomide, vorinostat, daunorubicin, pemetrexed, mesoridazine, mepacrine, cerulenin, idarubicin, carbamazepine and thalidomide*

Category CC5

Tretinoin and Tranylcypromine

Compounds marked with an asterisk (*) are not compounds of the invention but may be used in combination with a compound of the invention.

Compounds from 2 or 3 or 4 or 5 different categories may be used in combination.

In one embodiment of the invention, the compound selected from Compounds 1 to 203 is used in combination with a second or further compound selected from chloroquine, tacrolimus, thalidomide, dexamethasone and sirolimus. Suitably, the compound selected from Compounds 1 to 203 is used in combination with a second or further compound selected from chloroquine, tacrolimus, thalidomide, dexamethasone and sirolimus. Suitably, the compound selected from Compounds 1 to 203 is selected from Table 2. Suitably, the compound selected from Compounds 1 to 203 is selected from Table 3. Suitably, the compound selected from Compounds 1 to 203 is selected from Table 4. Suitably, the compound selected from Compounds 1 to 203 is selected from Table 5. In one embodiment, the compound selected from Compounds 1 to 203 is selected from the group consisting of PD-0325901, sulforaphane, neratinib, curcumin, trifluoperazine, olaparib, valproic-acid, fostamatinib, selumetinib, tivozanib, tozasertib, calcitriol, lovastatin, tamoxifen, NVP-AUY922, vemurafenib, nicardipine, promazine, orteronel and atorvastatin. Suitably, the compound selected from Compounds 1 to 203 is PD-0325901, sulforaphane, neratinib, cur cumin or trifluoperazine.

In one embodiment of the invention, PD-0325901 is used in combination with a compound selected from chloroquine, tacrolimus, thalidomide, dexamethasone and sirolimus. In one embodiment of the invention, sulforaphane is used in combination with a compound selected from chloroquine, tacrolimus, thalidomide, dexamethasone and sirolimus. In one embodiment of the invention, neratinib is used in combination with a compound selected from chloroquine, tacrolimus, thalidomide, dexamethasone and sirolimus. In one embodiment of the invention, curcumin is used in combination with a compound selected from chloroquine, tacrolimus, thalidomide, dexamethasone and sirolimus. In one embodiment of the invention, trifluoperazine is used in combination with a compound selected from chloroquine, tacrolimus, thalidomide, dexamethasone and sirolimus.

When the compounds selected from Compounds 1 to 203 are used in combination with second or further compounds of the invention, or with other compounds e,g, those mentioned above, the compounds of the combination may be administered either sequentially or simultaneously by any convenient route.

In one embodiment of the invention, a first compound selected from Compounds 1 to 203 and prodrugs thereof and pharmaceutically acceptable salts and solvates thereof is used in combination with a second or further compound selected from Compounds 1 to 203 and prodrugs thereof and pharmaceutically acceptable salts and solvates thereof, wherein the first and the second or further compounds are selected from the group consisting of calcium channel blockers, antihistamines (histamine receptor antagonist), statins, glucocorticoids and anti-psychotics.

In one embodiment of the invention, fluvoxamine is used in combination with a compound selected from nifedipine, felodipine, desloratadine, promethazine and atorvastatin.

In one embodiment, nifedipine is used in combination with promethazine.

In one embodiment, felodipine is used in combination with a compound selected from promethazine and desloratadine. In one embodiment, felodipine is used in combination with promethazine and fluvoxamine.

In one embodiment, atorvastatin is used in combination with a compound selected from nifedipine, felodipine, desloratadine and promethazine. In one embodiment, atorvastatin is used in combination with promethazine and fluvoxamine.

In one embodiment, hydrocortisone is used in combination with a compound selected from fluvoxamine, nifedipine, felodipine, desloratadine, promethazine and atorvastatin.

The compounds of the aforementioned specific combinations may be used in the form of prodrugs or salts or solvates.

The combinations referred to above may conveniently be presented for use in the form of a pharmaceutical formulation. However, the individual components of such combinations may be administered either sequentially or simultaneously in separate or combined pharmaceutical formulations. The individual components of combinations may also be administered separately, through the same or different routes.

Further therapeutic agents which may be used in combination with the compounds or combinations of the present invention include NSAIDs (such as aspirin, naproxen, ibuprofen, parecoxib, diclofenac), antipyretics such as paracetamol, pregabalin, gabapentin or opioids (such as fentanyl, sufentanil, oxycodone, morphine, tramadol, codeine).

### EXAMPLES

### Example 1

Overview

We have developed a proprietary methodology to represent phenotypic changes in normal and disease states using functional genomics data. This unique technology enables us to define disease stages and the transition paths between them. Our optimized prediction engine enables us to identify treatment strategies for alleviating pathological perturbations and restoring healthy states. Using our system, we can locate links between targets, mechanisms, indications and compounds and so generate effective and often unexpected treatment strategies.

Our systems pharmacology platform starts with generating phenotypic functional omics data (Epigenetic, Transcriptomics, Proteornics, Metabolomics). Using our methodology, we then transform this raw data into a new representation (“manifold”) to capture nonlinear dynamical changes underlying the biological states. These manifolds provide a map of omics data to a graph data structure that enables us to identify phenotypic changes in normal and disease states in terms of defined biological processes and pathways.

We Compile Manifolds into Two Databases:

1-Disease Manifold Database (DMD): uniquely represents phenotypic disease states before, during and after disease progression. For each state of a disease, the manifold provides a weighted list of perturbed processes and pathways.

To build the disease manifold that enables us to study the host response to CARS CoV-2 in this invention, we used primary human bronchial epithelial cells (NHBE).

Thus, normal human bronchial epithelial (NHBE) cells (Lonza, CC-2540 Lot #580580) were isolated from a 79-year-old Caucasian female and were maintained in bronchial epithelial growth media (Lonza, CC-3171) supplemented with BEGM SingleQuots as per the manufacturer's instructions (Lonza, CC-4175) at 37° C. and 5% CO2. SARS-CoV-2 was propagated in Vero E6 cells in DMEM supplemented with 2% FBS, 4.5 g/L D-glucose, 4 mM L-glutamine, 10 mM NonEssential Amino Acids, 1 mM Sodium Pyruvate and 10 mM HEPES. Infectious titers of SARS-CoV-2 were determined by plaque assay in Vero E6 cells in Minimum Essential Media supplemented with 4 mM L-glutamine, 0.2% BSA, 10 mM HEPES and 0.12% NaHCO₃ and 0.7% agar. Total RNA from infected and mock infected cells was extracted using TRIzol Reagent (Invitrogen) and Direct-zol RNA Miniprep kit (Zymo Research) according to the manufacturer's instructions and treated with DNase I. RNA-seq libraries of polyadenylated RNA were prepared using the TruSeq RNA Library Prep Kit v2 (Illumina) according to the manufacturer's instructions. RNA-seq libraries for total ribosomal RNA-depleted RNA were prepared using the TruSeq Stranded Total RNA Library Prep Gold (Illumina) according to the manufacturer's instructions. cDNA libraries were sequenced using an Illumina NextSeq 500 platform. RNA-Seq of viral infections with SARS-CoV-2 were performed at an MOI of 0.2 for 24 h in DMEM supplemented with 2% FBS, 4.5 g/L D-glucose, 4 mM L-glutamine, 10 mM NonEssential Amino Acids, 1 mM Sodium Pyruvate and 10 mM HEPES. Approximately 1×105 NHBE cells were infected with SARS-CoV-2 at an MOI of 2 for 24 h in bronchial epithelial growth media supplemented with BEGM SingleQuots.

After generating the transcriptional data for both infected and non-infected cells, we built the corresponding manifolds. These manifolds as a novel graph data structure enable us to build a weighted list of perturbed processes and pathways that uniquely represent the effect of COVID-19 on airway epithelial cells after infection.

2—Compound Manifold Database (CMD): uniquely represents phenotypic disease responses to a wide range of perturbations, including the effects of compounds before and after treatment. In order to build a comprehensive compound manifold database, a large number of compound libraries were treated on a number of selected cell lines that represent various diseases. After the treatment, the functional omics data including epigenetic, transcriptomics, proteomics or metabolomics were generated before and after treatment for each compound based on various doses and after specific time points. Using this functional data, we then generated corresponding manifold for each drug that provides a weighted list of processes and pathways that the corresponding drug can perturb.

These two proprietary databases enabled us to build a comprehensive disease-drug database of unprecedented size and functionality. Using the this database, we built and optimised a Search Engine powered by a unique ranking system to 1) rank novel unexpected strategies to modulate diseases (e.g. ranking biological processes and pathways), and 2) rank the corresponding compounds that deliver the desired modulations and will alleviate the pathological perturbations causing disease.

For COVID-19, we adapted our platform by combining our biological and clinical knowledge. We selected lung as the most affected tissue and created a manifold for it based on pre and post infections states. We aimed to find single and combination compound treatments which could be used to treat COVID-19.

We then optimised the ranking system of our prediction engine to identify candidate compounds for alleviating pathological perturbations caused by COVID-19 and restoring the healthy state. The engine performs a comprehensive search against our compound manifold database. This enables us to find unexpected links between targets, mechanisms, and compounds that promise the greatest possibility for developing therapeutics for this disease.

We researched each top-ranked compound and excluded certain compounds on a range of criteria. Candidate compounds deemed to have excellent potential are selected for experimental validation.

Results:

The results of our DMD generation was a list of the most significant processes modulated by COVID-19. These were ranked by the extent of the modulation between healthy cells and COVID-19 infected cells.

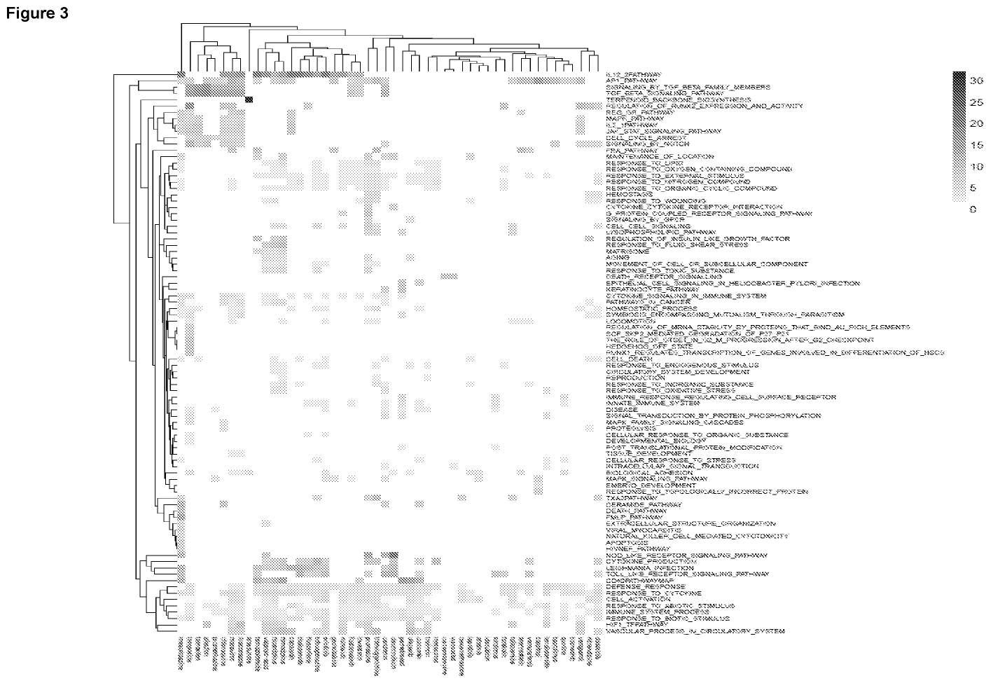

FIG. 1 summarises the suppression capability of 23 compounds from our CMD. The COVID-19 manifold provides the modulated biological processes and pathways after the infection. Top 50 of them are ranked on one axis from highest activation (cytokine and inflammatory response) to least activation (TGF beta signalling pathway). The suppression capability of each compound on the other axis is shown, with strongest suppression response black and dark grey and weakest response light grey and white. FIGS. 2 and 3 can be interpreted similarly and relate to other compounds of the invention and certain prior art compounds.

FIGS. 1-3 show that the cytokines and inflammatory response was the most perturbed by COVID-19, while the TGF beta signalling pathway was the lowest ranked response still deemed biologically significant for our project.

Considering the COVID-19 manifold and corresponding modulated biological process and pathways, we believe that results for COVID-19 will extend to treatments for lung inflammation and modulation of immune system response more generally.

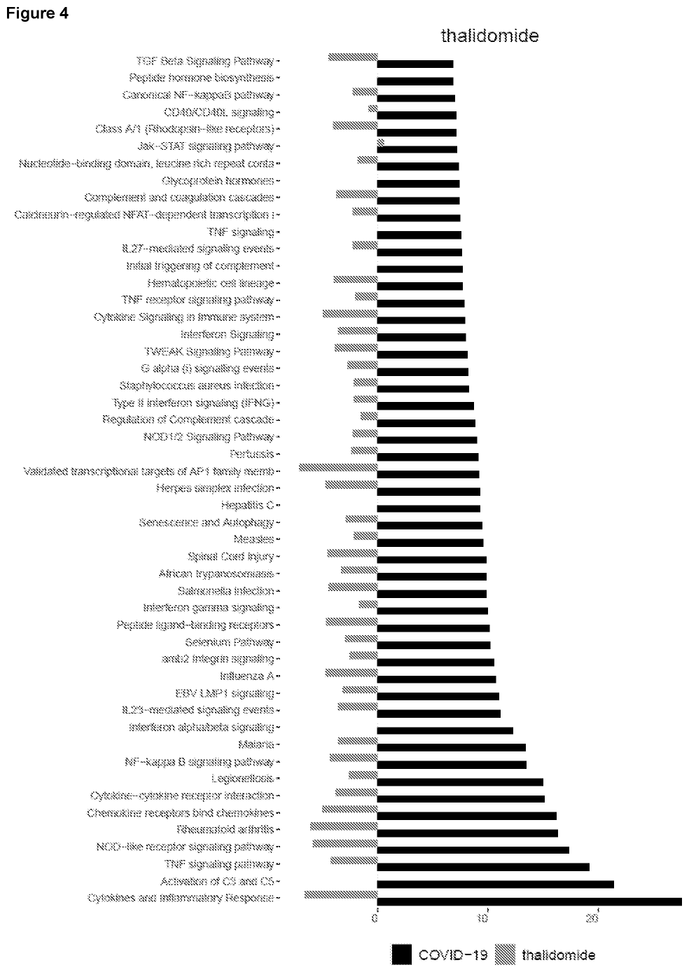

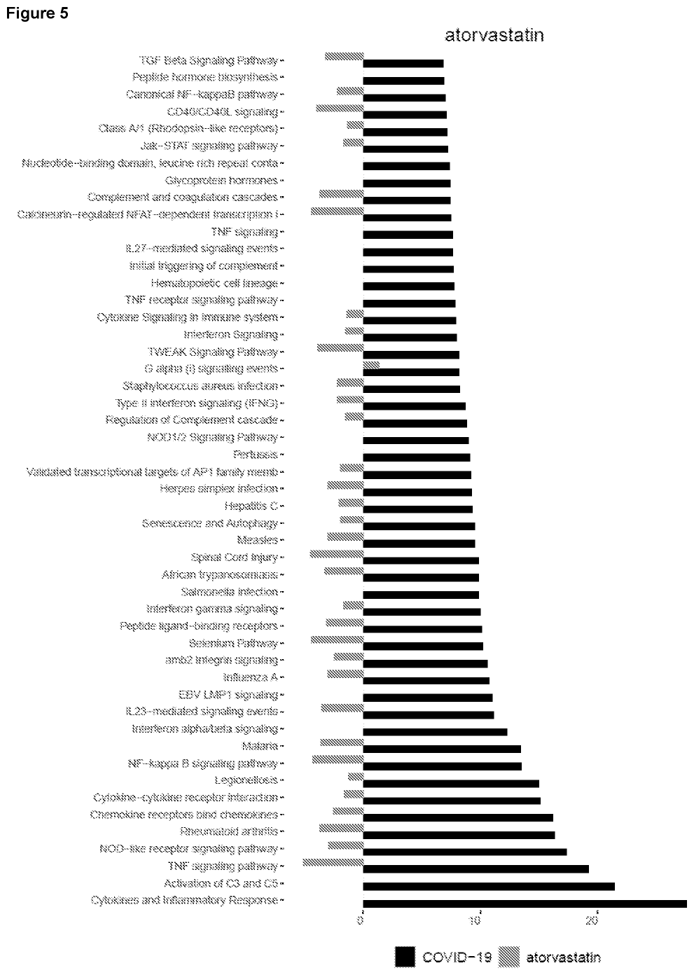

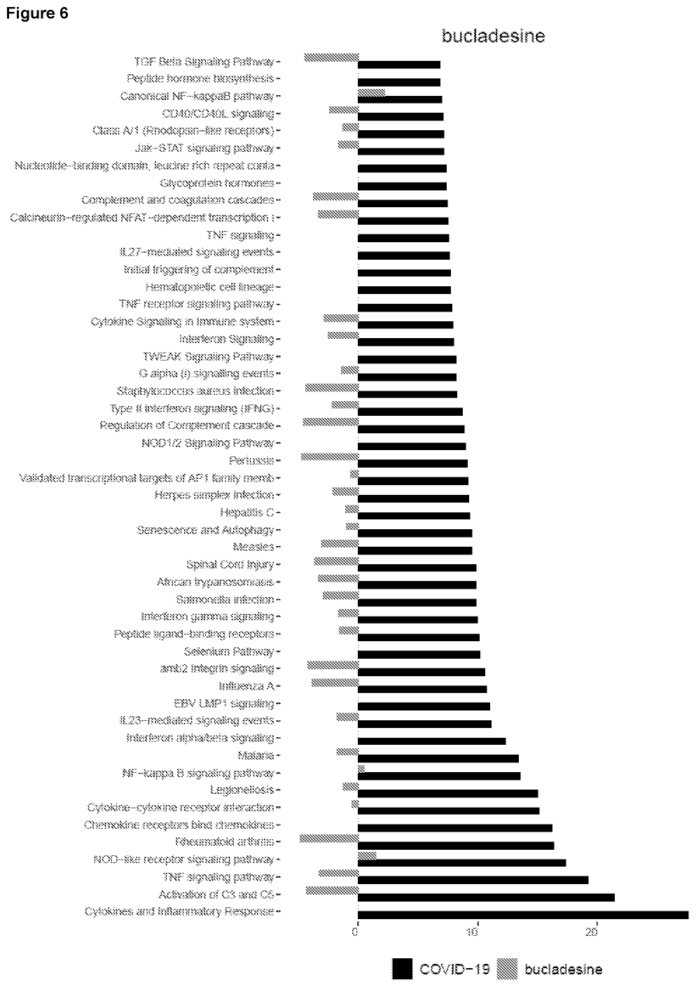

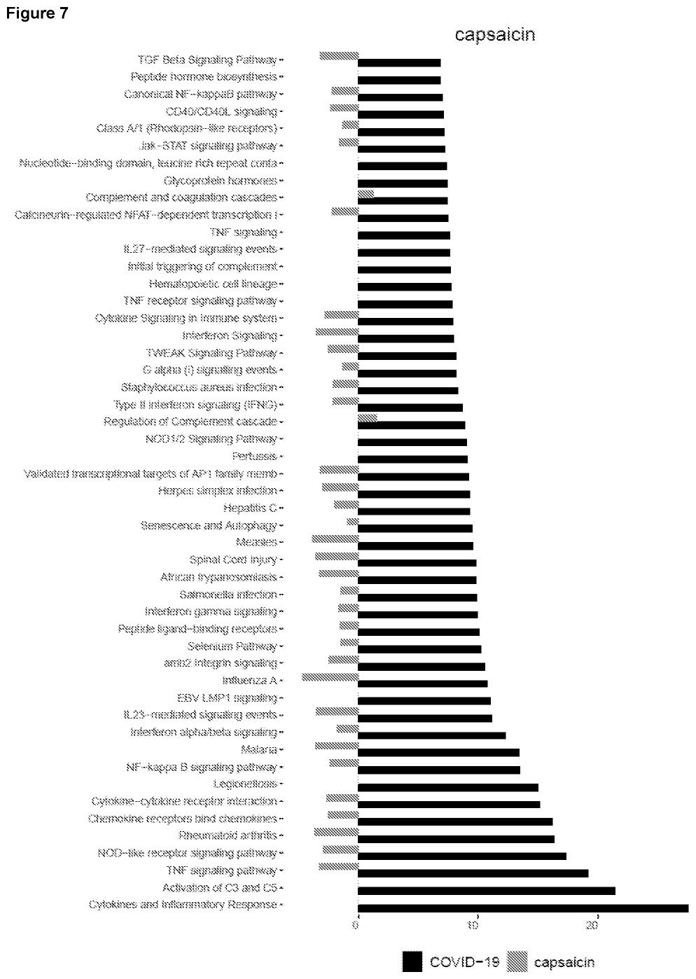

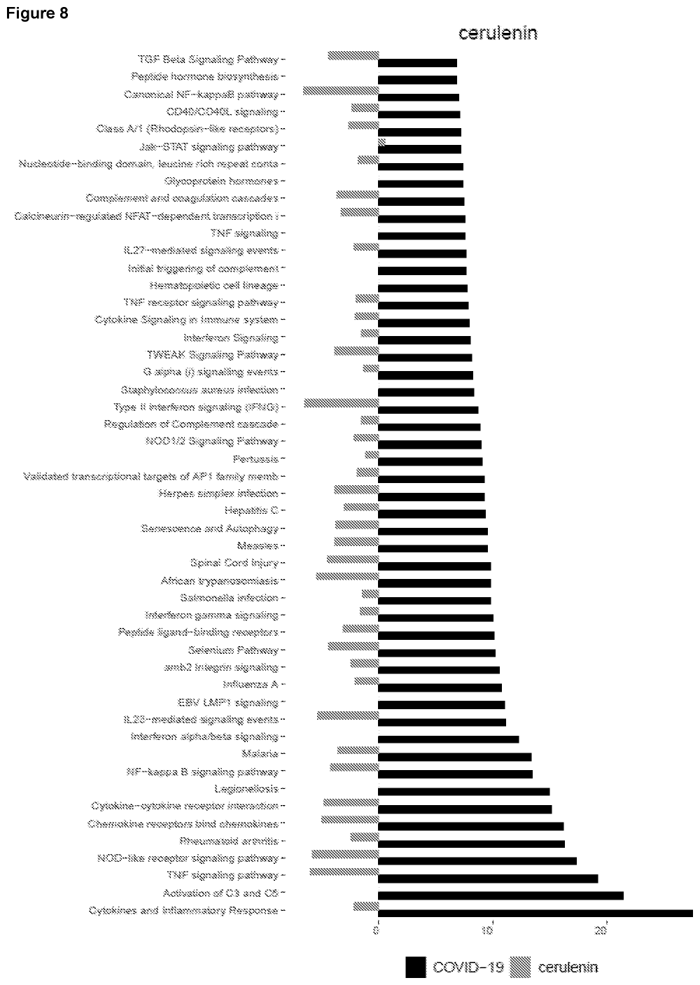

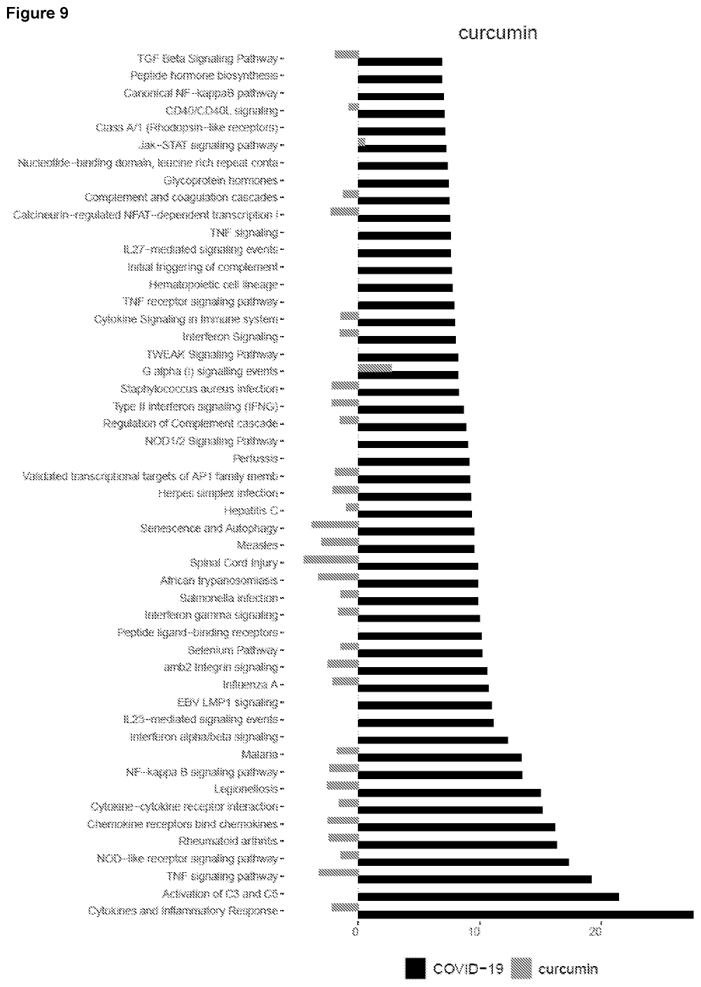

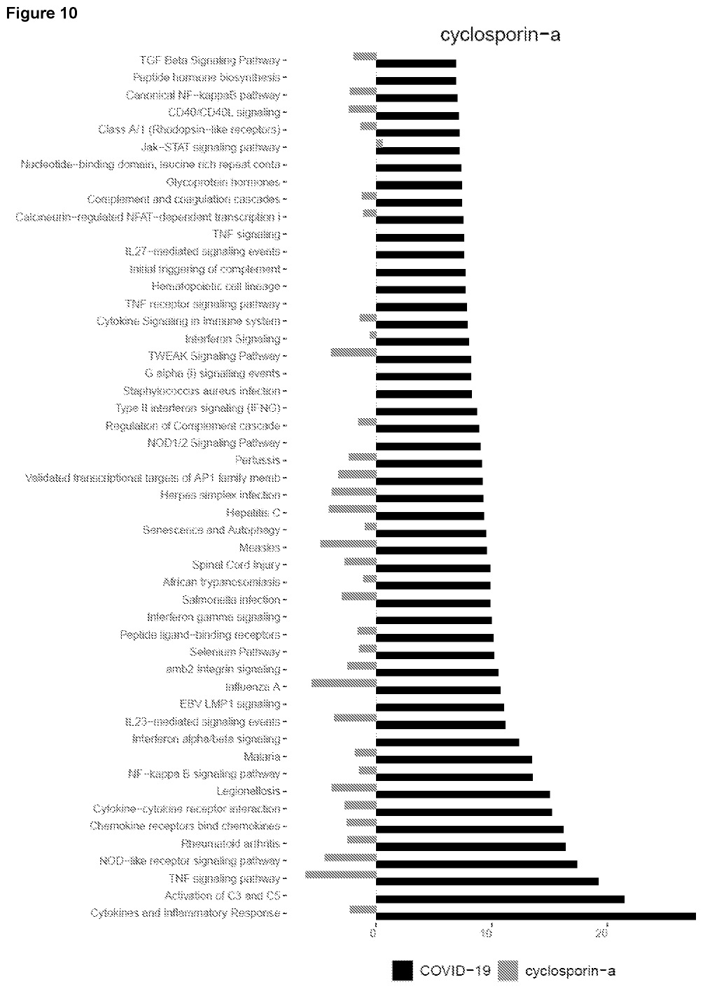

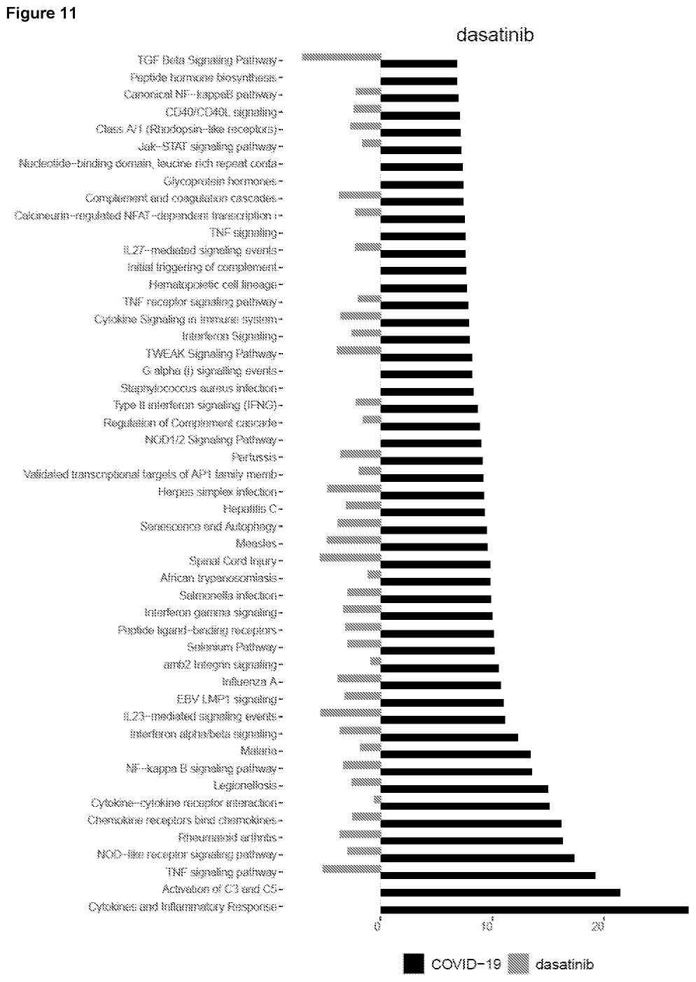

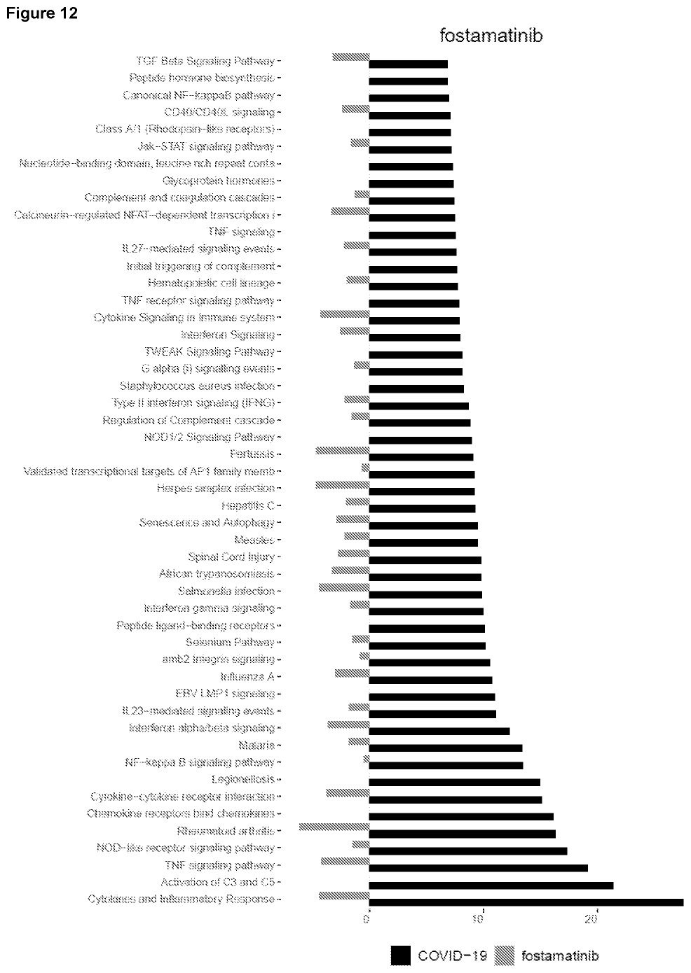

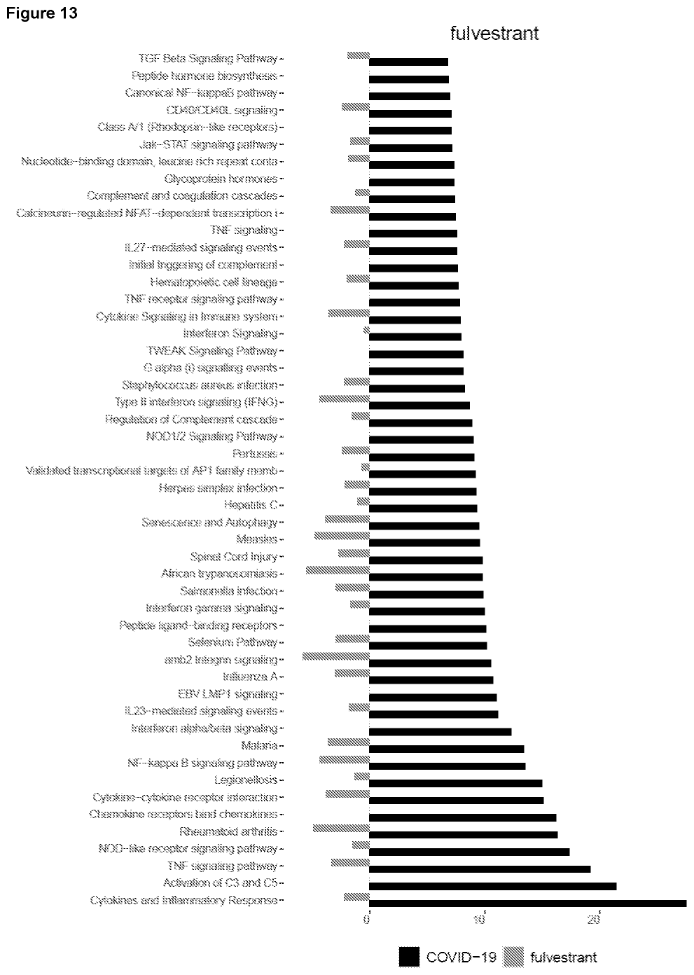

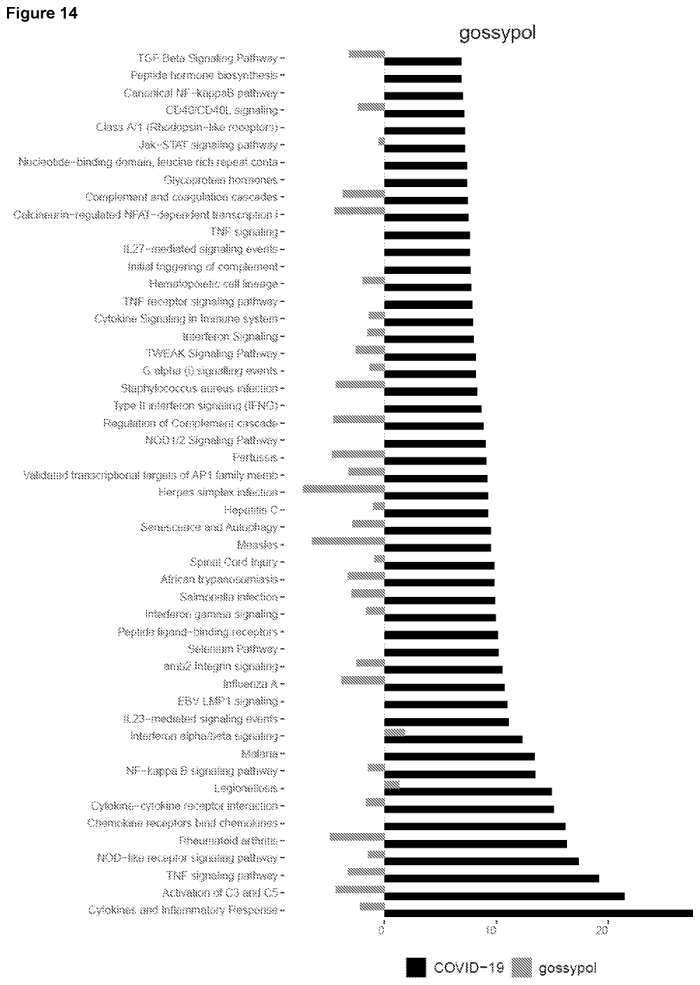

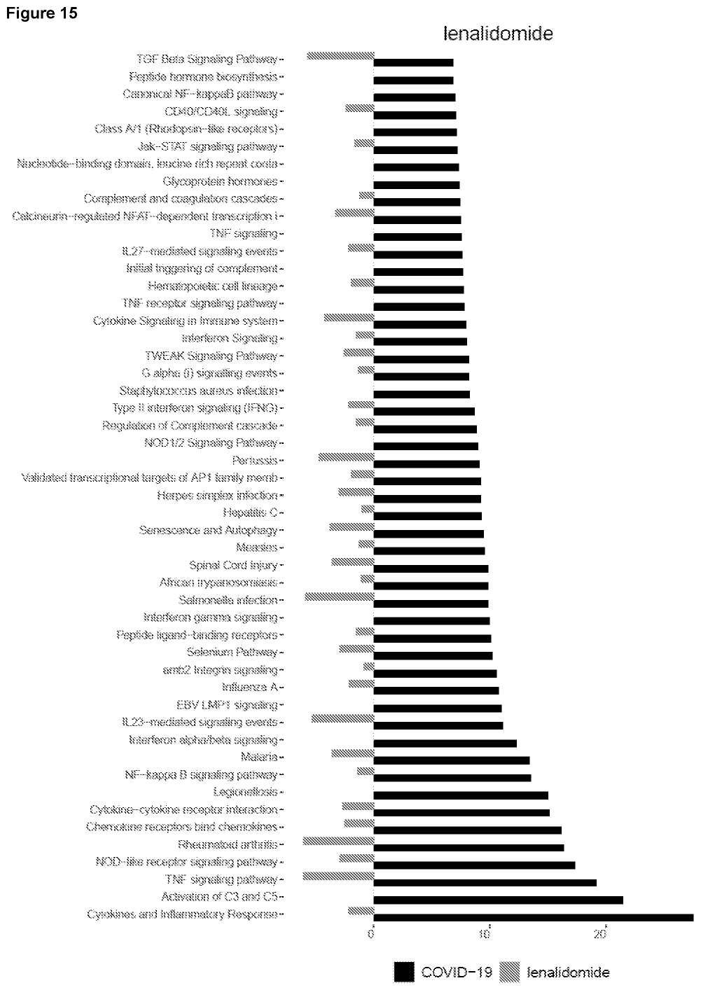

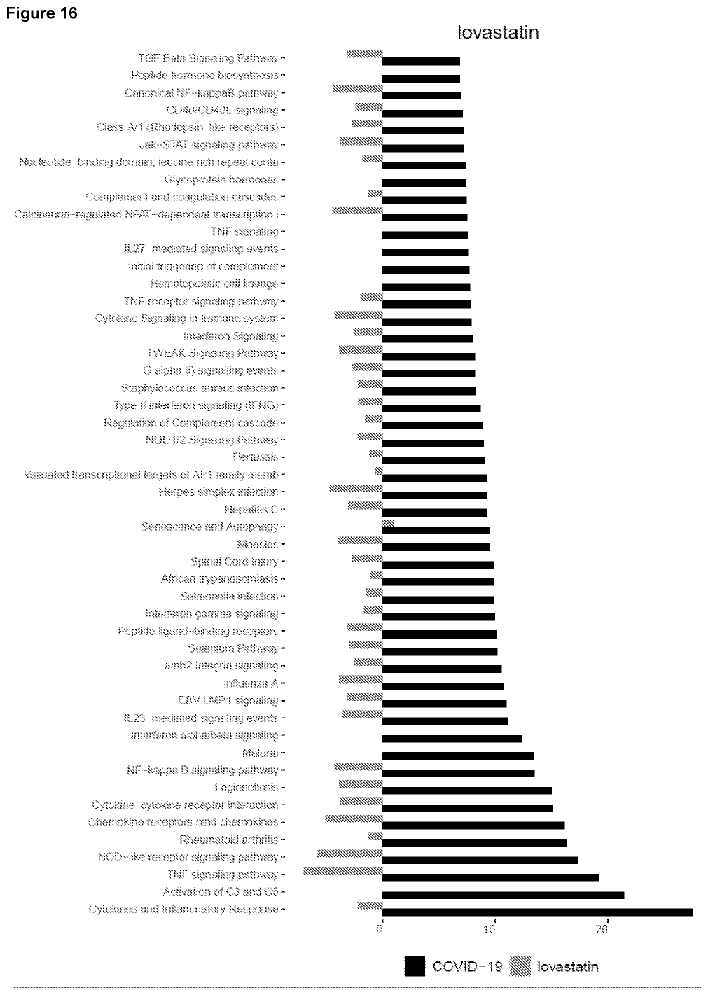

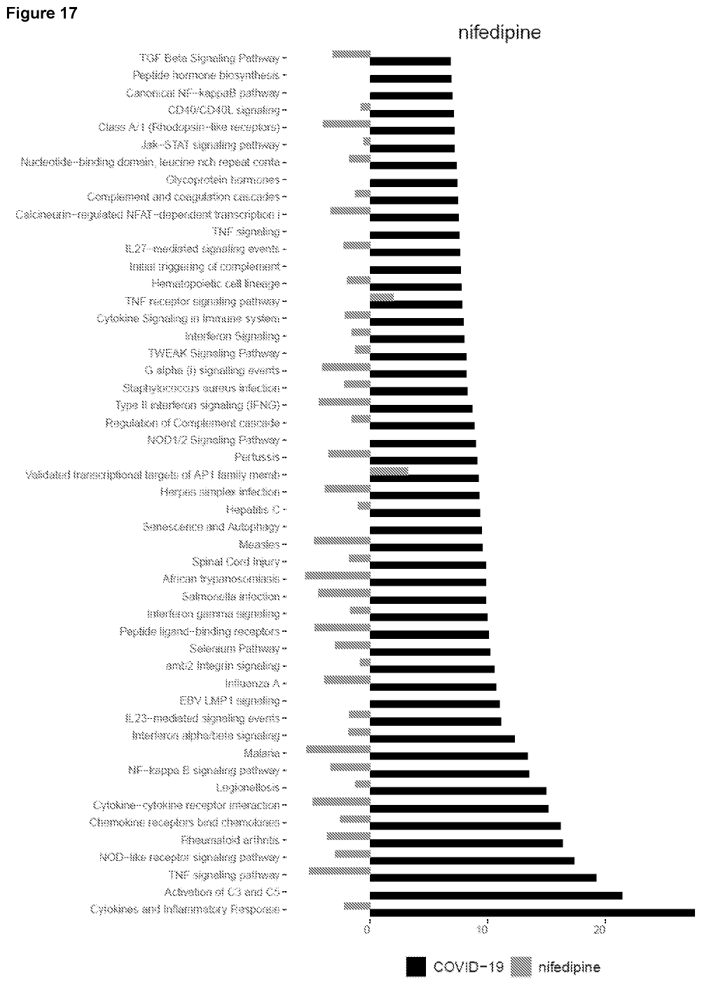

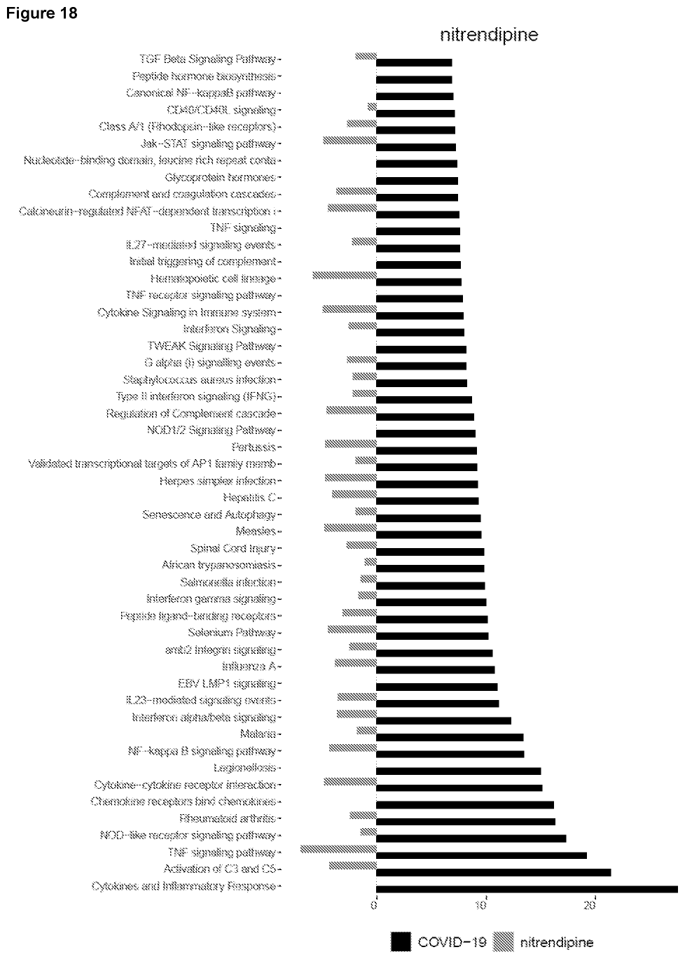

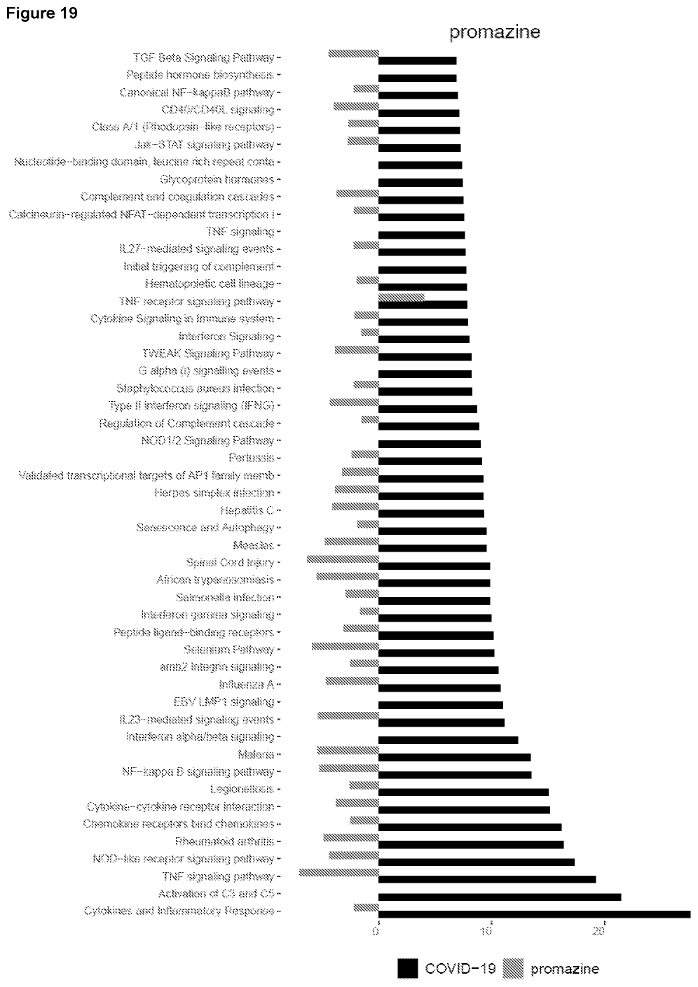

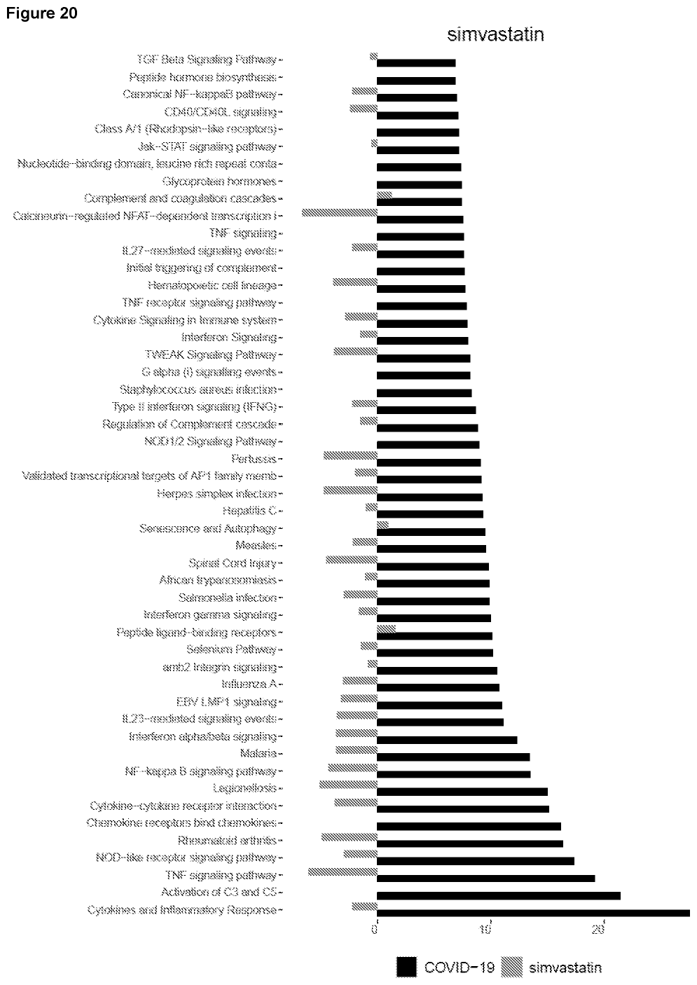

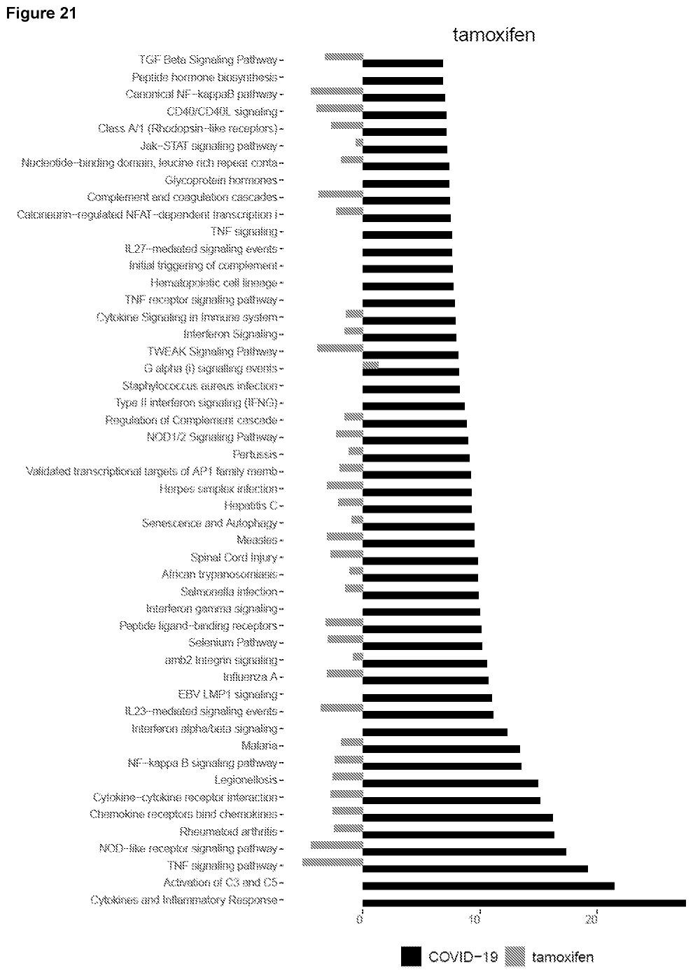

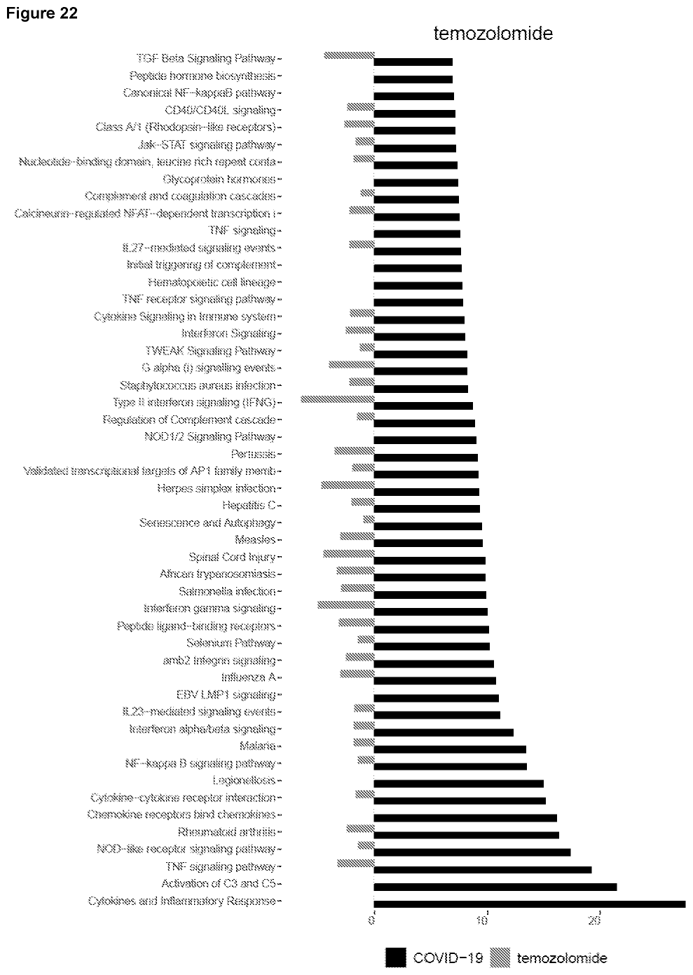

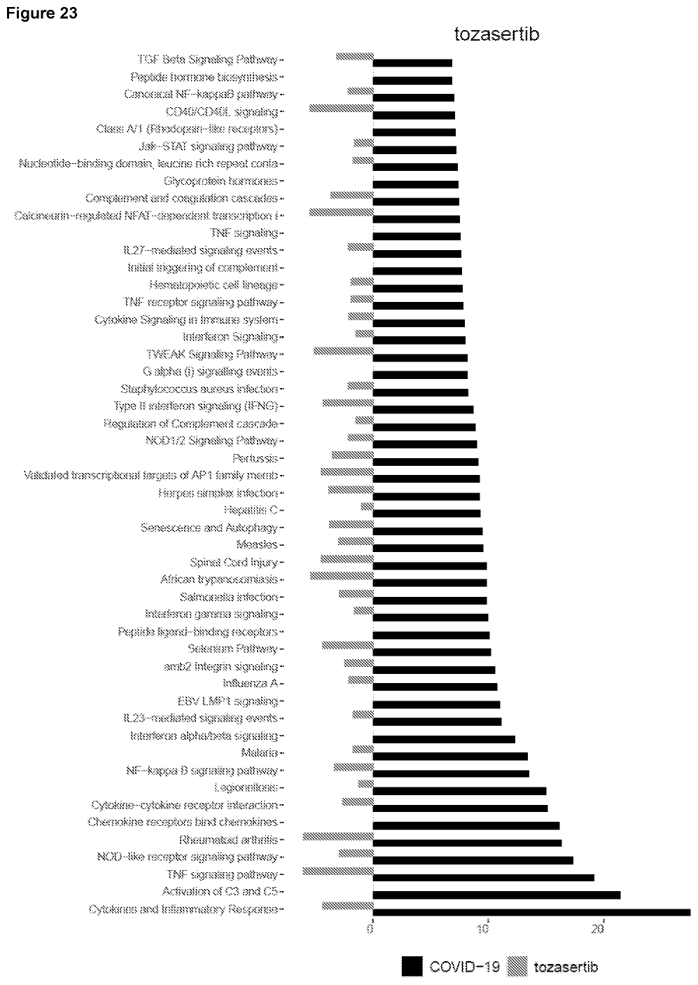

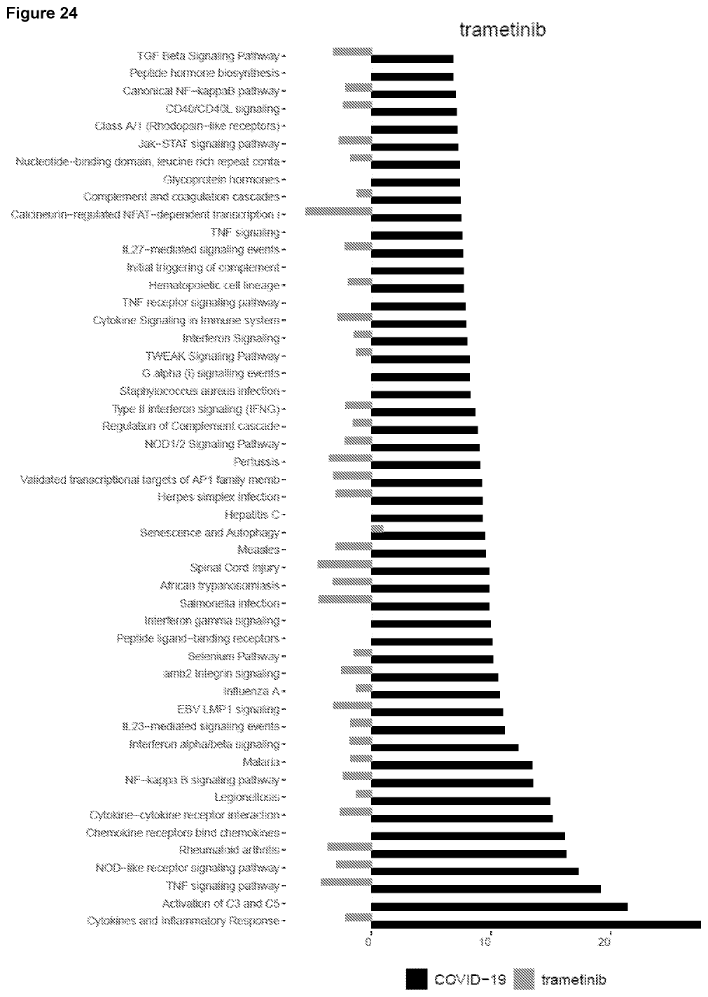

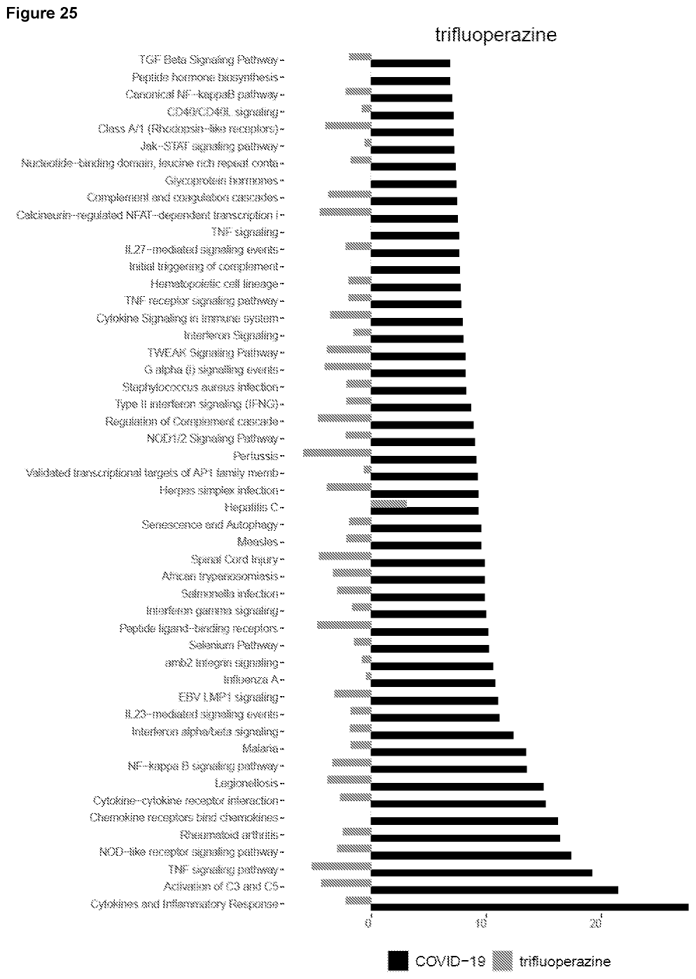

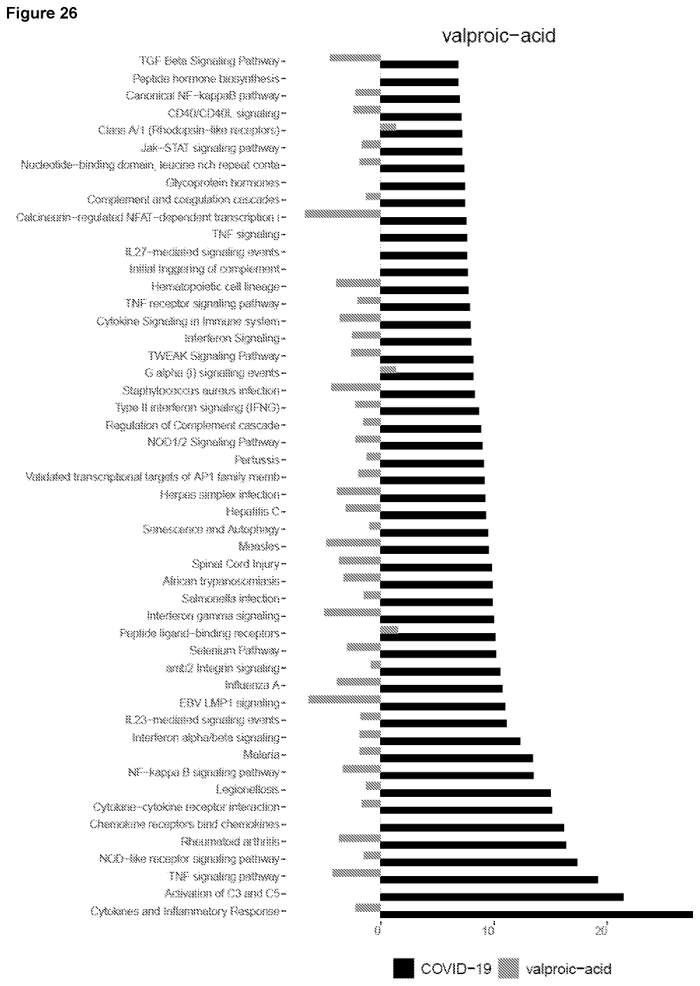

The goal of our prediction engine was to search our CMD to find the compounds which we predict will most strongly suppress COVID-19 perturbations, prompting the suppression of the strongest COVID-19 responses phenotypically. As explained above, for each drug in our CMD, we produce a modulation signature for all of the processes and pathways modulated by COVID-19. A variant representation of the information of FIGS. 1-3 is shown in FIGS. 4-26 for the compounds specified. This will be explained for illustrative purposes by reference to FIGS. 4 and 11. FIG. 4 and FIG. 11 provides sample signatures for the compounds thalidomide and dasatinib, respectively. While the COVID-19 manifold ranking on the x-axis is identical in both examples, we can see that thalidomide and dasatinib are predicted to act differently on each COVID-19 response (y-axis). Therefore, they provide different phenotypic responses as they modulate different sets of processes and pathways. The black bars show the strength of the COVID-19 perturbations for corresponding processes and pathways, while the grey bars show the strength of the compound's suppression of the corresponding processes and pathways. For example, thalidomide is predicted to suppress the COVID-19 cytokines and inflammatory response (FIG. 4), while dasatinib is not (FIG. 11). Thalidomide is also predicted to cause greater suppression of the COVID-19 cytokine-cytokine receptor interaction than dasatinib.

In post-processing, individual compounds of the invention are further investigated. Compounds chosen for wet-lab validation experiments (Example 3) demonstrate not only strong predicted ability to suppress COVID-19 responses, but also predicted suitability to complement and augment other compounds within a possible drug combination.

The compounds of the invention and particular compounds of the invention which result from our analysis are listed in Tables 6, 7 and 8.

TABLE 6
List of candidate compounds (compounds of the invention)

| Comp. | |
|---|---|
| No | Compound Name |
| 1 | pravastatin |
| 2 | 17-hydroxyprogesterone-caproate |
| 3 | acrivastatin |
| 4 | alprenolol |
| 5 | amiloride |
| 6 | amitriptyline |
| 7 | amoxapine |
| 8 | atorvastatin |
| 9 | atovaquone |
| 10 | betamethasone |
| 11 | bezafibrate |
| 12 | budipine |
| 13 | calcipotriol |
| 14 | capsaicin |
| 15 | carbenoxolone |
| 16 | chlorphenamine |
| 17 | ciclosporin |
| 18 | clomethiazole |
| 19 | clomifene |
| 20 | clozapine |
| 21 | curcumin |
| 22 | cyclosporin-a |
| 23 | diazepam |
| 24 | enoxacin |
| 25 | estradiol |
| 26 | esmolol |
| 27 | etofylline clofibrate |
| 28 | estriol |
| 29 | flunisolide |
| 30 | felodipine |
| 31 | flecainide |
| 32 | hydrocortisone |
| 33 | fluphenazine |
| 34 | fluvoxamine |
| 35 | fostamatinib |
| 36 | linezolid |
| 37 | isoxsuprine |
| 38 | labetalol |
| 39 | lenalidomide |
| 40 | mazindol |
| 41 | lobeline |
| 42 | loratadine |
| 43 | lovastatin |
| 44 | maprotiline |
| 45 | methoxsalen |
| 46 | mepyramine |
| 47 | naphazoline |
| 48 | neostigmine |
| 49 | pimavanserin |
| 50 | nicergoline |
| 51 | nifedipine |
| 52 | nitrendipine |
| 53 | norethindrone |
| 54 | paclitaxel |
| 55 | palonosetron |
| 56 | raloxifene |
| 57 | pindolol |
| 58 | prednisone |
| 59 | proguanil |
| 60 | promazine |
| 61 | saxagliptin |
| 62 | repaglinide |
| 63 | rivaroxaban |
| 64 | rosuvastatin |
| 65 | salmeterol |
| 66 | sulpiride |
| 67 | scopolamine |
| 68 | simvastatin |
| 69 | ticagrelor |
| 70 | tamoxifen |
| 71 | valsartan |
| 72 | triamcinolone |
| 73 | trifluoperazine |
| 74 | trimethobenzamide |
| 75 | trimipramine |
| 76 | ubenimex |
| 77 | valproic-acid |
| 78 | zonisamide |
| 79 | ibudilast |
| 80 | desloratadine |
| 81 | cefoxitin |
| 82 | ornidazole |
| 83 | erythromycin |
| 84 | mitotane |
| 85 | pazopanib |
| 86 | temozolomide |
| 87 | trametinib |
| 88 | cilnidipine |
| 89 | dilazep |
| 90 | naftidrofuryl |
| 91 | fulvestrant |
| 92 | mestranol |
| 93 | bucladesine |
| 94 | spiperone |
| 95 | metergoline |
| 96 | niacin |
| 97 | dihydroergocristine |
| 98 | ondansetron |
| 99 | tolterodine |
| 100 | rho-kinase inhibitor |
| 101 | thioridazine |
| 102 | 616-dimethylprostag |
| 103 | 6-aminochrysene |
| 104 | cp72-4714 |
| 105 | gossypol |
| 106 | trichostatin-a |
| 107 | tozasertib |
| 108 | dppe |
| 109 | ib-meca |
| 110 | indole-3-carbinol |
| 111 | remacemide |
| 112 | saracatinib |
| 113 | dasatinib |
| 114 | cerulenin |
| 115 | batimastat |
| 116 | nvp-auy922 |
| 117 | canertinib |
| 118 | afatinib |
| 119 | vorinostat |
| 120 | erlotinib |
| 121 | 3-amino-benzamide |
| 122 | veliparib |
| 123 | linsitinib |
| 124 | carbamazepine |
| 125 | px-12 |
| 126 | idarubicin |
| 127 | foretinib |
| 128 | mk-1775 |
| 129 | tipifarnib |
| 130 | elesclomol |
| 131 | gdc-0941 |
| 132 | flutamide |
| 133 | ibrutinib |
| 134 | nintedanib |
| 135 | rilmenidine |
| 136 | temsirolimus |
| 137 | etoposide |
| 138 | tivantinib |
| 139 | sulforaphane |
| 140 | selumetinib |
| 141 | tivozanib |
| 142 | calcitriol |
| 143 | vemurafenib |
| 144 | promethazine |
| 145 | artesunate |
| 146 | minoxidil |
| 147 | fk-866 |
| 148 | triptolide |
| 149 | daunorubicin |
| 150 | genistein |
| 151 | tg-101348 |
| 152 | mepacrine |
| 153 | pemetrexed |
| 154 | forskolin |
| 155 | gemcitabine |
| 156 | alvocidib |
| 157 | tretinoin |
| 158 | fluspirilene |
| 159 | orantinib |
| 160 | tanespimycin |
| 161 | buparlisib |
| 162 | sn-38 |
| 163 | bortezomib |
| 164 | equol |
| 165 | clonidine |
| 166 | navitoclax |
| 167 | quinine |
| 168 | zileuton |
| 169 | verapamil |
| 170 | maraviroc |
| 171 | teniposide |
| 172 | rucaparib |
| 173 | mk-2206 |
| 174 | itraconazole |
| 175 | neratinib |
| 176 | pd-0325901 |
| 177 | olaparib |
| 178 | orteronel |
| 179 | nicardipine |
| 180 | nimodipine |
| 181 | pelitinib |
| 182 | pd-184352 |
| 183 | dalcetrapib |
| 184 | mesoridazine |
| 185 | tolazamide |
| 186 | etomoxir |
| 187 | lapatinib |
| 188 | rolipram |
| 189 | bi-2536 |
| 190 | enzalutamide |
| 191 | bms-754807 |
| 192 | resveratrol |
| 193 | tak-715 |
| 194 | nilotinib |
| 195 | losartan |
| 196 | estrone |
| 197 | imiquimod |
| 198 | enmd-2076 |
| 199 | cp-724714 |
| 200 | decitabine |
| 201 | ex-527 |
| 202 | phenethyl-isothiocyanate |
| 203 | tranylcypromine |
| — | — |

TABLE 7
Suitable compounds of the invention

| | |
|---|---|
| PD-0325901 | zileuton |
| sulforaphane | lenalidomide |
| neratinib | promethazine |
| curcumin | canertinib |
| trifluoperazine | afatinib |
| olaparib | vorinostat |
| valproic-acid | PD-184352 |
| fostamatinib | triptolide |
| selumetinib | temozolomide |
| tivozanib | daunorubicin |
| tozasertib | genistein |
| calcitriol | verapamil |
| lovastatin | artesunate |
| tamoxifen | minoxidil |
| NVP-AUY922 | FK-866 |
| vemurafenib | erlotinib |
| nicardipine | dasatinib |
| promazine | veliparib |
| orteronel | TG-101348 |
| atorvastatin | mepacrine |
| nimodipine | maraviroc |
| batimastat | teniposide |
| navitoclax | 3-amino-benzamide |
| EX-527 | pemetrexed |
| quinine | mesoridazine |
| pelitinib | dalcetrapib |

TABLE 8
Other suitable compounds of the invention.

| | |
|---|---|
| atorvastatin | trifluoperazine |
| capsaicin | valproic-acid |
| curcumin | temozolomide |
| cyclosporin-a | trametinib |
| fostamatinib | fulvestrant |
| lenalidomide | bucladesine |
| lovastatin | gossypol |
| nifedipine | tozasertib |
| nitrendipine | dasatinib |
| promazine | cerulenin |
| simvastatin | — |
| tamoxifen | — |

### Example 2 Combination Therapies

Combination therapies exploit the chances for better efficacy, decreased toxicity, and reduced development of drug resistance and owing to these advantages. For COVID-19 and related diseases combination therapy represents a promising approach. In this context, studying the effects of a combination of drugs in order to provide evidence of a significant superiority compared to the single agents is of particular interest.

Using the previously discussed manifolds, compounds of the invention were clustered into different classes. By clustering the selected compounds based on their manifold we were able to cluster the compounds into various different categories (e.g. C1, C2, C3, C4, C5, CC1, CC2, CC3, CC4 and CC5). In each category, there are a number of compounds which display similar graph data structure in their manifold and modulate similar sets of biological processes and pathways phenotypically. Therefore, the selection of compounds which derive from different categories results in modulation of the COVID-19 manifold from different angles concurrently. This may improve the probability of a synergistic combination. The selection of two compounds from the same category may also provide stronger perturbation of the same processes and pathways. Consequently, using this clustering approach on manifolds provide a novel strategy to choose drug combination with phenotypic synergy. The same approach can be used to find combinations of compounds wherein each compound limits undesirable modulation of the other.

From our disease and compound modelling we were able to model and predict a number of such combinations, as shown in FIGS. 27 and 28. The compounds of the invention and particular compounds which result from our analysis are listed in Tables 9 and 10.

TABLE 9
Further suitable compounds of the invention

| | |
|---|---|
| neratinib | EX-527 |
| sulforaphane | NVP-AUY922 |
| PD-0325901 | pelitinib |
| olaparib | lenalidomide |
| trifluoperazine | zileuton |
| valproic-acid | promethazine |
| curcumin | PD-184352 |
| fostamatinib | triptolide |
| selumetinib | minoxidil |
| lovastatin | genistein |
| tamoxifen | verapamil |
| tozasertib | canertinib |
| tivozanib | afatinib |
| calcitriol | vorinostat |
| nicardipine | temozolomide |
| batimastat | FK-866 |
| orteronel | daunorubicin |
| atorvastatin | artesunate |
| nimodipine | TG-101348 |
| navitoclax | maraviroc |
| promazine | veliparib |
| quinine | teniposide |
| vemurafenib | dasatinib |

TABLE 10
Additional suitable compounds of the invention

| | |
|---|---|
| olaparib | daunorubicin |
| trifluoperazine | artesunate |
| valproic-acid | maraviroc |
| curcumin | teniposide |
| fostamatinib | dasatinib |
| lovastatin | erlotinib |
| tamoxifen | pemetrexed |
| calcitriol | mesoridazine |
| nicardipine | mepacrine |
| atorvastatin | tolazamide |
| nimodipine | cerulenin |
| promazine | trametinib |
| quinine | tretinoin |
| vemurafenib | idarubicin |
| lenalidomide | gemcitabine |
| zileuton | forskolin |
| promethazine | capsaicin |
| minoxidil | lapatinib |
| verapamil | carbamazepine |
| afatinib | itraconazole |
| vorinostat | nitrendipine |
| temozolomide | fluspirilene |
| — | tranylcypromine |

The compounds of Table 9, together with prior art compounds tacrolimus, chloroquine, dexamethasone and sirolimus, were mapped to show which other compounds in the set showed similar predicted activities. 5 categories of compounds were identified, labelled C1 to C5 (see FIG. 27).

Category C1

Fostamatinib, zileuton, artesunate, tacrolimus* and TG-101348.

Category C2

Neratinib, PD-0325901, olaparib, trifluoperazine, valproic-acid, tozasertib, tivozanib, calcitriol, nicardipine, orteronel, nimodipine, promazine, promethazine, PD-184352, minoxidil, canertinib, temozolomide and selumetinib.

Category C3

Lovastatin, tamoxifen, atorvastatin, navitoclax, EX-527, chloroquine*, verapamil, afatinib, maraviroc, teniposide and dasatinib.

Category C4

Sulforaphane, curcumin, dexamethasone*, sirolimus*, batimastat, quinine, vemurafenib, pelitinib, lenalidomide, triptolide, genistein, vorinostat, FK-866, daunorubicin, veliparib and selumetinib.

Category C5

NVP-AUY922

The compounds of Table 10, together with prior art compounds tacrolimus, chloroquine, dexamethasone, sirolimus and thalidomide, were mapped to show which other compounds in the set showed similar predicted activities. 5 categories of compounds were identified, labelled CC1 to CC5 (see FIG. 28).

The Categories are Set Out Below:

Category CC1

Fostamatinib, zileuton, artesunate, tacrolimus* and trametinib

Category CC2

Olaparib, trifluoperazine, valproic-acid, calcitriol, nicardipine, nimodipine, promazine, promethazine, minoxidil, temozolomide, erlotinib, tolazamide, lapatinib, fluspirilene and thalidomide*

Category CC3

Lovastatin, tamoxifen, atorvastatin, chloroquine*, verapamil, afatinib, maraviroc, teniposide, dasatinib, gemcitabine, forskolin, capsaicin, itraconazole and nitrendipine

Category CC4

Curcumin, dexamethasone*, sirolimus*, quinine, vemurafenib, lenalidomide, vorinostat, daunorubicin, pemetrexed, mesoridazine, mepacrine, cerulenin, idarubicin, carbamazepine and thalidomide*

Category CC5

Tretinoin and Tranylcypromine

Compounds marked with an asterisk (*) are not compounds of the invention.

### Example 3 Experimental Validation

In vitro tests for anti-COVID-19 activity on the candidate compounds selected in Example 1 are performed as follows:

Drug screening is performed on monolayer airway epithelium cell lines. We also validate the drug activities using 3D air-liquid interface (ALI) cultures of well-differentiated primary airway epithelial cells.

The human airway epithelium constitutes the first line of defence against respiratory pathogens, air pollution, and allergens. Studying how these environmental insults affect the airway tissue and developing effective treatments to combat them requires a physiologically relevant model system.

Primary human bronchial epithelial cells (HBECs) cultured at the Air-Liquid Interface (ALI) undergo extensive mucociliary differentiation that recapitulates what is observed in vivo. As a result, ALI culture of HBECs is increasingly being recognized as the most physiologically relevant in vitro model system for respiratory research.

Conventional submerged culture systems do not adequately capture the complex morphological and functional characteristics of the in vivo human airway, and animal primary cell models can have limited experimental windows and lack perfect homology to human systems. Addressing these limitations, air-liquid interface (ALI) culture of human primary airway epithelial cells is being increasingly adopted as a model system for the in vivo human airway epithelium.

For the study of respiratory viruses, such as the novel SARS-CoV-2 virus that causes COVID-19, ALI cultures are advantageous because they preserve key characteristics of the in vivo airway epithelium that viruses target. Viruses evolve in parallel to their host cells and, consequently, show pronounced specificity for both host species and cell type. Infection models for a given virus must therefore be specific to and closely representative of the native cells it targets. To date, the ALI culture system has been used to study a wide range of viruses, including the novel coronavirus.

The physiological relevance of the ALI culture system is supported by phenotypical and histological evidence. Hematoxylin and eosin (H&E) and periodic acid-Schiff (PAS) staining show that HBECs cultured at the ALI are pseudostratified in morphology and make up a heterogeneous cell population that includes ciliated and mucus-secreting (PAS-positive) cells, similar to the in vivo bronchial epithelium. An important function of the tracheobronchial epithelium in vivo is to act as a protective barrier against inhaled insults. The same epithelial barrier function has been confirmed in ALI cultures by the expression of tight junction proteins and the development of high transepithelial electrical resistance. The suitability of ALI cultures for modelling the airway has been further confirmed through transcriptome analyses and a wide range of experiments demonstrating physiological responses to insults such as toxicants and pathogens. Importantly, ALI cultures of primary cells from donors with respiratory disease (e.g. asthma, cystic fibrosis, COPD) recapitulate in vivo disease characteristics, forming robust in vitro models of these conditions.

In order to isolate and identify COVID-19, we infect ALI cultures of pathogen-free human airway epithelial cells with fluid from patients showing symptoms. This human airway infection model allows us to isolate the virus that is reproducibly infecting human lung cells.

Several existing methods utilize bronchial epithelial cells, which can be obtained invasively via brush biopsies during bronchoscopies, or from otherwise discarded lung tissue. In addition, commercial sources to obtain fully differentiated human airway epithelial cell cultures exist (EpiAirway model from MatTek, Ashland, Mass., Clonetics from Lonza, Basel, Switzerland, MucilAir from Epithelix Sars, Geneva Switzerland), where you can choose from different donors. However, because of the limited pool of commercially available epithelial cells, limited or prescribed set of parameters, the cost associated with commercially obtaining primary human airway ECs, and the limited access to discarded lung tissue or freshly obtained human bronchial biopsy tissue, prohibit us from conducting studies in fully differentiated human airway ECs. Therefore, we use a technique that will 1) utilize non-invasively obtained human NECs, 2) provide a protocol yielding cultures of differentiated human airway epithelium, and 3) be reproducible in most lab settings with an existing tissue culture infrastructure.

1. Prepare Plastic Ware: Coat Plate
1. Add 500 μl PureCol (1:100 diluted with sterile water) to each well of a 12-well plate.
2. Incubate at 37° C. in an incubator for 30 min, remove the PureCol and rinse with HBSS right before the cells are added to the plate (see step 3.1 below).

2. Obtaining and Pretreating Biopsy of Human NECs
1. Nasal scrape biopsy
   1. Obtain superficial ECs lining the nasal turbinates under direct vision through a 9 mm reusable polypropylene nasal speculum (Model 22009) on an operating otoscope with speculum (Model 21700). This device provides optimal visualization of the nasal turbinates and flexibility of motion.
   2. Perform the biopsies with the subject seated upright in a straight-backed chair with head tilted back slightly or lying in a supine position on an examination table.
   3. Insert the otoscope speculum into the nostril and the inferior turbinate visualized with illumination.
   4. Insert a sterile thermoplastic curette through the speculum with the tip extended distally to the back of the turbinate.
   5. Using gentle pressure on the inferior surface of the turbinate, the curette is drawn across the mucosal surface 5× and retracted. A successful retrieval of mucosal cells held by capillary action will be evident in the cup of the curette.
2. Place sample in a 15 ml conical tube containing 8 ml of RPMI 1640, transport on ice.
3. Use forceps to retrieve the probe, dislodge tissue from rhino probe with a P-1000 pipette, discard probe
4. Centrifuge to pellet (4° C., 400×g, 5 min), aspirate supernatant (attention: careful not to aspirate the pellet).
5. Resuspend the pellet in 1 ml BEGM with 10 μl of 100×DNase 1.
6. Incubate at RT for 20 min.
7. Centrifuge (4° C., 400×g, 5 min), do NOT aspirate the supernatant!

3. Seed the Cells on Plastic (Day 0)
1. Remove the PureCol from the plate and rinse with HBSS (see also step 1.2).
2. Add a minimum volume of BEGM++ media (80-100 μl, until a meniscus of media appears in the center of the well) into 1-4 wells (depending on biopsy size) of the coated plate.
3. Use a P-1000 pipette to carefully remove the pellet of the biopsy tissue from the tube, without aspirating too much media.
4. Transfer the pellet into the middle of the wells, keep the biopsy together as a cluster of cells, and do not break up to make a single cell suspension. A good biopsy yields up to 4 wells that can be seeded. Put the plate into tissue culture incubator (37° C., 5% CO₂, >90% relative humidity).
5. Check after two hours to assure that there is enough media (without disturbing the meniscus).
6. Day 1, 24 hr post-seeding: collect all non-adherent cells of all wells (tilt plate and collect media with cells in a P-1000, collect all wells in a 1.5 ml centrifuge tube).
7. Centrifuge (4° C., 400× g, 5 min).
8. While centrifuging cell suspension: add about 250 μl of BEGM++ and 250 μl of BEGM+ media mixture to the cells seeded on day 0.
9. Repeat steps 3.1-3.5 for the non-adherent cells.
10. Day 2-5: change media of the cells daily; add 500 μl media to each well, reduce the amount of BEGM++ media stepwise (day 2 after seeding: 125 μl BEGM++ plus 375 μl BEGM+, day3 after seeding and the following days: 73 μl BEGM++ plus 427 μl BEGM+).

4. Expanding the Cells in the Flasks
1. Monitor cells daily; once they have reached 80-90% confluency (usually 5-7 days after the biopsy, if they take longer they won't grow successfully) lift cells as following: carefully aspirate media; add 500 μl of 0.25% trypsin per well, keep them at 37° C. until the cells are detached (it usually takes 2-3 min).
2. Prepare the needed volume of Soy Bean Trypsin Inhibitor (SBTI) (750 μl per 1 ml Trypsin) in a 15 ml tube.
3. Dislodge cells by repeatedly pipetting up and down the trypsin solution; transfer detached cells in the 15 ml conical tube with SBTI.
4. Rinse all wells with in total 1 ml HBSS, add HBSS to the tube with the cells.
5. Centrifuge to pellet (4° C., 400× g, 5 min), aspirate the media, resuspend in 1 ml BEGM+ media and vortex the tube.
6. Expand cells in a T25 or T75 flask depending on pellet size (this is p1; approximately two confluent wells go into one T75 flask, one confluent well is enough for one T25, usually 2 confluent wells yield one T75).
   1. Add BEGM+ to the flasks (5 ml each for a T75, 3 ml each for a T25).
   2. Add the cell suspension to the flasks. Transfer the flasks into a tissue culture incubator.
7. 24 hr post-flask-seeding: add additional BEGM+ media to the flask (3 ml to a T25, 10 ml to a T75).
8. maintain the cells in the flasks: Remove media (aspirate from the corner) and add BEGM+ media (5 ml/T25, 20 ml/T75). The media needs to be changed every 2-3 days. Maintain the cells in the flask until they are about 80% confluent (confluency may not be even throughout the entire flask; normally this takes between 7-10 days; if they are not confluent after 10 days they won't grow successfully).

5. Seed the Cells on Tissue Culture Inserts
1. Remove the media from the flask and add Trypsin (3 ml for T25, 4 ml for T75 flask).
2. Incubate at 37° C. until the cells are detached (about 2-3 min).
3. Prepare SBTI (750 μl per 1 ml Trypsin >2.25 ml for T25, 3 ml for T75) in a 15 ml tube.
4. Dislodge cells by taping the flask and pipetting up and down and transfer to conical tube with the SBTI.
5. Rinse the flask with HBSS (1 ml for T25, 2 ml for T75), add the HBSS to the 15 ml tube.
6. Centrifuge to pellet (4° C., 400× g, 5 min), remove supernatant carefully.
7. Prepare the plates: decide how many plates are going to be seeded, label the plates with sample code and number, passage number (i.e. p2) and date. Add 700 μl BEGM+ media to the basal chamber.
8. Resuspend the cell pellet in 1 ml BEGM ALI media by vortexing the tube.
9. Count the cells with a hemocytometer (a T25 usually produces about 4 million cells; a T75 can have up to 20 million cells depending on the islet; use 1-2 million of cells for one 12-well plate) and dilute the cell suspension with BEGM ALI media to the total volume needed for the amount of plates that should be seeded (1.2×10⁶ cells in 2.5 ml media per plate).(Note: If part of the cells want to be frozen, label the tube and remove cells here: we usually freeze 2×10⁶ cells in 2 ml of freezing media)
10. Add 200 μl cell suspension to the apical side of each uncoated Corning 12well cell insert (>this will be about 80,000-150,000 cells/insert; 12 mm transwells with 0.4 μm pore polyester (Polyethylene Terephthalate (PET)) membrane insert); transfer into tissue culture incubator.
11. After 24 hr, change the apical media (200 μl expansion medium).
12. Maintaining the cell cultures: Change the media (BEGM ALI media; 700 μl basolateral and 200 μl apical) every other day. Inserts have to be maintained submerged until cells are totally confluent (no holes in monolayer are visible). Depending on cell number this usually takes between 2-6 days, if it takes longer the cells will most likely not be viable.

6. Establish Air Liquid Interface Once the Cells are Completely Confluent (When the Cells on the Transwells are Completely Confluent)
1. Once the cells are completely confluent replace both apical and basolateral media with BEGM ALI media supplemented with 500 nM retinoic acid for 48 hr.
2. 48 hr after adding the retinoic acid supplemented BEGM ALI media: Prepare PneumaCult-ALI Maintenance Medium (following manufacturer's instructions): Calculate the volume of medium needed (for one plate usually 8.5 ml for basal compartments); add per 1 ml PneumaCult Complete Base Medium:
   10 μl PneumaCult 100× Maintenance Supplement
   2 μl Heparin (of a 2 mg/ml stock solution)
   5 μl Hydrocortisone (of a 96 μl/ml stock solution).
3. Remove all media and add 700 μl PneumaCult-ALI maintenance media to the basolateral side only (no medium on the apical side).
4. Maintain the cell cultures for 4 weeks at the ALI:
5. Change the media every other day
6. After one week at ALI, once a week (Wednesdays) the apical surface is rinsed: add 200 μl HBSS to the apical side, keep them for 10 min in the incubator, carefully aspirate the HBSS (use a 9 in glass pipette attached to the vacuum trap in the Biological safety cabinet, use 200 μl tips on the tip of the pipette to suction off the HBSS).
7. Change tips between plates, as well as changing the tips when going from apical surface versus basolateral. After about 2 weeks at the ALI, cells start to differentiate and cilia appear. Cell cultures reach full differentiation after about 4 weeks at the ALI.

Screening of Candidate Compounds May Include:
EC₅₀ and CC₅₀ determinations of test compounds (8 doses, in duplicates)+
+ reference compound
Evaluation of the combination effect of compound pairs (7*7 doses, in triplicates)
EC₅₀ and CC₅₀ determinations of test compounds in cell-based qPCR assays (8 doses, in duplicates)
Evaluation of the combination effect of compound pairs against COVID-19 in cell-based qPCR assays (7*7 doses, in triplicates)
collect the cultures from the cellular assays and analyse against a cytokine panel by a multiplex Luminex assay.

### Example 4: In Vitro Studies

List of Reference Compounds

| Reference | |
|---|---|
| Compound | Compound Name |
| 1 | chloroquine |
| 2 | spermidine |
| 3 | niclosamide |
| 4 | pyronaridine |
| 5 | hydroxychloroquine |
| 6 | remdesivir |
| 7 | amlodipine |
| 8 | albuterol |
| 9 | cetirizine |
| 10 | dexamethasone |
| 11 | methylprednisolone |
| 12 | betaferon |
| 13 | bromhexine |
| 14 | doramapimod |
| 15 | BAPTA-AM |
| 16 | (2-aminoethyl diphenylborinate (2APB))) |
| 17 | lopinavir |
| 18 | ritonavir |
| 19 | azithromycin |
| 20 | fluvastatin |
| 21 | pitavastatin |

Anti-Viral Drugs
1. Anti-Hypertensive Drugs

The anti-viral efficacy of calcium channel blockers (CCBs) (class of anti-hypertensive drugs) Compound 51 (nifedipine) and Compound 30 (felodipine) was tested on Vero E6 and Calu-3 cells (as referred to in R. Straus et al. 2020)). The cells were treated with serial concentrations of drug compounds and infected with SARS-CoV-2 at an MOI of 0.05. At 24 hpi, viral copy number in the supernatant was measured with qRT-PCR and cell viability was measured with CCK-8 assay.

Results

The results are summarised in Table 11:

TABLE 11
Results of the study

| | | | | SI = |
|---|---|---|---|---|
| Compound | Cell Line | EC₅₀ (uM) | CC₅₀ (uM) | CC50/EC50 |
| Compound 30 | Calu-3 | 0.01255 | 122.8 | 9784.86 |
| (Felodipine) | | | | |
| Compound 51 | Calu-3 | 20.47 | ND* | ND* |
| (Nifedipine) | | | | |

*ND = not determined

Compound 51 (nifedipine) reduced the viral titers by 1.5 logs at a concentration of 100 μM and no virus was detectable at 500 μM while cytotoxicity was moderate. Indeed, because Compound 51 (nifedipine) did not show a significant cytotoxic effect at approx. 7× the concentration of the most efficacious antiviral concentration of 300 μM a SI value could not be determined. Compound 30 (felodipine) diminished SARS-CoV-2 growth by half at 10 μM and at 50 μM no virus was detected with no cytotoxic effect on the cells. Further studies demonstrated that Reference Compounds 15 (BAPTA-AM) and 16 (2-Aminoethyl Diphenylborinate (2APB))) significantly inhibit SARS-CoV-2 replication in a concentration dependent manner, confirming the dependence role of intracellular Ca2+ for SARS-CoV-2 replication and the antiviral effect of CCBs (data not shown).
2. Anti-Psychotic Drugs

Compound 33 (fluphenazine), Compound 158 (fluspirilene)

Compound 33 (fluphenazine) and Compound 158 (fluspirilene) were selected to investigate the anti-viral effect of anti-psychotic drugs against SARS-CoV-2 (as referred to in Weston et al., 2020). Vero E6 cells were grown in 24 well plate format for 24 h prior to infection. The cells were pre-treated with the Compound 33 or 158, or vehicle control for 2 h and then then infected with SARS-CoV-2 at MOI 0.01 or 0.004 for 24 hour (h) for Vero E6.

Results

Table 12 illustrates the average 1050 and CC50 values for the drugs against SARS-CoV-2 in Vero cells. The results showed the antiviral effect of these groups of antipsychotic drugs.

TABLE 12
Results of the study

| | | | | | Selectivity |
|---|---|---|---|---|---|
| Compound | cell line | MOI | IC₅₀ (uM) | CC₅₀ (uM) | Index (SI) |
| Compound 158 | Vero-E6 | 0.004 | 6.36 | 30.33 | 9.61 |
| (fluspirilene) | | 0.01 | 5.32 | 30.33 | >5.71 |
| Reference | Vero-E6 | 0.004 | 42.03 | >>50 | 1.19 |
| Compound 1 | | 0.01 | 46.8 | >>50 | 1.07 |
| (chloroquine) | | | | | |
| Compound 33 | Vero-E6 | 0.004 | 6.36 | 20.02 | 3.15 |
| (fluphenazine) | | 0.01 | 8.98 | 20.02 | 2.23 |

The results showed the efficacy of the Compounds 33 (fluphenazine) and 158 (fluspirilene) against SARS-CoV-2 is higher than Reference Compound 1 in both MOI: 0.004 and 0.01. Additionally, this ability of SARS-CoV-2 inhibition was at the range of non-cytotoxic concentrations.

Compound 73 (Trifluoperazine)

Human adenocarcinomic alveolar basal epithelial (A549) cells (ATCC, CCL-185) were maintained at 37° C. and 5% CO2 in Dulbecco's Modified Eagle Medium (DMEM, Gibco) supplemented with 10% Fetal Bovine Serum (FBS, Corning) (as outlined in Hoagland et al., 2020). A549^{AcE2} heterogeneous cell population was generated by transducing A549 cells with lentivirus without selection. Compound 73 treatment were administered to plated cells and cell cultures 1 hour prior to infection.

Results

The in vitro results showed trifluoperazine in both pre-treted Vero-E6 and A549^{AcE2} cells successfully blocked SARS-COV-2 replication where vRNA levels quantified by qRT-PCR analysis of infected cells after 24 hours of SARS-CoV-2 infection at MOI of 0.01. (Significance compared to DMSO treated values and determined by unpaired two-tailed student's t-test, p<0.001, n=3 biological replicates) for RT-qPCR (Hoagland et al., 2020).
3. Anti-Histamine Drugs

Compound 144 (Promethazine) and Compound 16 (Chlorphenamine)

The anti-viral effects of Compound 144 (promethazine), Compound 16 (chlorphenamine) and Reference Compound 1 (chloroquine) were tested on Vero E6 cells (as referred to in Hoagland et al., 2020). The cells were grown in 24 well plate format for 24 h prior to infection. The cells were pre-treated with Compounds 144, 16 or Reference Compound 1, or vehicle control for 2 h and then then infected with SARS-CoV-2 at MOI 0.01 or 0.004 for 24 hour (h) for Vero E6 or 48 h for A549-hACE2 cells

Result

Table 13 illustrates the average IC50 and CC50 values for the drugs against SARS-CoV-2.

TABLE 13
Results of the study

| | | | | Selectivity |
|---|---|---|---|---|
| Compound | MOI | IC₅₀ (uM) | CC₅₀ (uM) | Index (SI) |
| Reference | 0.004 | 42.03 | >>50 | 1.19 |
| Compound 1 | 0.01 | 46.8 | >>50 | 1.07 |
| (chloroquine) | | | | |
| Compound 144 | 0.004 | 9.21 | >42.59 | 4.62 |
| (promethazine) | 0.01 | 10.44 | >42.59 | 4.08 |
| Compound 16 | 0.004 | 3.14 | 11.88 | 3.78 |
| (chlorphenamine) | 0.01 | 4.03 | 11.88 | 2.94 |

The results showed the antiviral effect of these groups of antihistamine drugs and the efficacy of against SARS-CoV-2 is higher than the refence compound in both MOI: 0.004 and 0.01 at the range of non-cytotoxic concentrations.

Compound 80 (desloratadine) and Compound 42 (loratadine)

In vitro SARS-CoV-2 spike pseudotyped viral infection experiments were performed (as referred to in Hou et al., 2020) using ACE2 overexpressing HEK293T cells (ACE2^{h}) to check the antiviral effect of Compound 80 (desloratadine) and Compound 42 (loratadine). Firstly, 5×10⁴ of ACE2h cells in 100 μL DMEM per well were seeded into white 96-well plates. The cells were cultured in a 37° C. incubator containing 5% CO2 for 24 h. Then 50 μL of medium was carefully aspirated from wells followed by adding another 50 μL of medium containing corresponding dose of Compound 80 or 42 and incubating for 2 h. 10 μL of SARS-CoV-2 Spike pseudotyped virus was then added. After infection at 37° C. with 5% CO2 in incubator for 10-12 h, the culture medium containing the virus was sucked away and replaced by 200 μL of fresh DMEM, and the cells were incubated continuously at 37° C. for 48 h. After that, the culture medium was aspirated. 20 μL of cell lysate and 100 μL of luminescence solution were added to each well before the luciferase luminescence detection by a microplate reader under 560 nm with exposure time as 1 s.

Results

The pseudotype entry results compared to control results are outlined in Table 14:

TABLE 14
Results of the study

| | pseudotype entry |
|---|---|
| Compound | (mean) |
| Control | 1 |
| Compound 42 | 0.7 |
| (loratadine) | |
| Compound 80 | 0.3 |
| (desloratadine) | |

The results indicate that that Compound 42 (loratadine) and Compound 80 (desloratadine) could prevent entry of the pseudotyped virus into HEK293T^{ACE2} cells. Further binding experiments using cell membrane chromatography and surface plasmon resonance demonstrated that both antagonists could bind to ACE2 and that the binding affinity of desloratadine was much stronger than that of loratadine. The results are consistent with the virucidal activity of Compound 16 (chlorphenamine maleate) that showed 99.7% reduction in the viral load (as referred to in Westover et al., 2020) and Compound 144 (promethazine) as SARS-CoV-2 entry inhibitor (as referred to in Yang et al., 2020)
4. RTK inhibitors and inhibitors of RTK pathway

Compound 112 (Saracatinib) and Compound 187 (Lapatinib)

Telomerase-immortalized MRCS fibroblasts (MRCS cells) were cultured in Dulbecco's modified Eagle serum (DMEM; Invitrogen) to investigate the effect of the FDA-approved drugs such as Compound 112 (saracatinib), Compound 187 (lapatinib) and Compound 118 (afatinib) (as referred to in Raymonda et al., 2020).

Viral Stocks of 0C43 were Propagated in MRCS Cells in 2% (Vol/Vol) FBS, 4.5 g/Liter Glucose, and 1% penicillin-streptomycin at 34° C. Viral stock titers were determined by TCID50 analysis, and for the assessment of OC43 viral replication, viral titer was determined via TCID50 analysis. MRCS cells were infected with OC43 at an MOI of 0.05 TCID50/mL. MRCS-ACE2 fibroblast were grown to confluence on a 6-well plate and then pretreated with media containing either

DMSO (0.25% Vol/Vol), Compound 187 (lapatinib) (5 μM), or Reference Compound 6 (remdesivir) (2.5 μM). After 4 hours, cells were either mock infected or infected with SARS-CoV-2, Isolate Hong KongNM20001061/2020 (BEI Resources NR-52282) at a MOI of 0.01. After a 1 hour adsorption period, viral inoculum was removed and replaced with media containing drug or DMSO. Samples were harvested in SDS-lysis buffer at 4 and 24 hours post infection. Percent inhibition of viral killing was determined as: (Cell Number Infected(Drug)—Average Cell Number Infected(DMSO)/(Average Cell Number Mock Infection(DMSO)—Average Cell Number Infected(DMSO))*100%. All values calculated to be negative were set to “0”.

Results

The maximal percent inhibition of viral killing across all concentrations is summarised in Table 15.

TABLE 15
Results of the study

| | | Max % Inhibition of |
|---|---|---|
| Compound | Target | Viral Killing |
| Compound 112 | BcrAbl | 22.48 |
| (saracatinib) | | |
| Compound 187 | EGFR | 63.99 |
| (lapatinib) | | |

The results showed that Compound 187 (lapatinib) strongly inhibited OC43 and SARS-CoV-2 viral replication with inhibition also observed with Compound 112 (saracatinib). Additionally, further investigations showed Compound 187 (lapatinib) inhibited SARS-CoV-2 replication by over 50,000-fold without any toxicity and at doses readily achievable in human tissues (data not shown). Indeed, the IC50 of Reference Compound 6 (remdesivir) against OC43-induced CPE was in the μM range, whilst co-treatment with Compound 187 (lapatinib) reduced the IC50 of remdesivir into the sub-μM range (data not shown).

Compound 173 (MK-2206)

The antiviral effect of Compound 173 (MK-2206) in Vero E6 cells was compared with Reference Compound 2 (spermidine) and Reference Compound 3 (niclosamide) (as referred to in Gassen et al., 2020). Cells were cultivated in Dulbecco's Modified Eagle's Medium (DMEM) supplemented with 10% fetal bovine serum (FBS), 1% 14 penicillin/streptomycin, 1% nonessential amino acids, and 1% sodium pyruvate at 37° C. and 5% CO2. For virus infection with SARS-CoV-2 strain Munich 984, 2×10e5 cells m1-1 were seeded in 6-well plates. After 24 h, cells were infected with an MOI=0.0005 in serum-free medium. In parallel, mock infected cells were inoculated with heat-inactivated (95° C., 10 min) virus. After 1 hour, the virus dilutions were removed and the wells were washed twice with PBS and refilled with DMEM. Samples were taken at the indicated time points (8, 24, and 48 hours).

Results

The antiviral effect of the compounds is summarized in Table 16:

TABLE 16
Results of the study

| | Reducing | | |
|---|---|---|---|
| | Viral | | |
| Compound | propagation | IC50 (uM) | |
| Compound 173 | 88% | 0.09 | (R² = 0.95) |
| (MK-2206) | | | |
| Reference Compound 2 | 85% | 149 | (R² = 0.71) |
| (spermidine) | | | |
| Reference Compound 3 | >99% | 17 | (R² = 0.63) |
| (niclosamide) | | | |

The results showed that Reference Compound 2 (spermidine), Compound 173 (MK-2206) and Reference Compound 3 (niclosamide) inhibited SARS-CoV-2 propagation by 85, 88, and >99%, respectively.

1. Estrogen receptor agonist The VERO E6 cells (ATCC® CRL-1586™) cell lines that normally express both nuclear (ERα, ERβ) and membrane estrogen receptors (GPER1) were used to check the effect of Compound 25 (estradiol) against SARS-CoV-2 infection (as referred to in Lemes et al., 2021). 1×10⁵ of cultured VERO E6 cells were previously treated with 17β-estradiol (E2) at 10⁻⁹M during 24 hours at 5% CO2 and 37° C. DMSO (at concentration 0.00001%) was used in parallel as vehicle control. For infection, estimated MOI (multiplicity of infection) of 0.2 of SARS-CoV-2, were diluted in 200 μL of DMEM F12 with 1% FBS and added or not (mock) to each treated cell-well condition and incubated for 2 hours at 5% CO₂ and 37° C.

Results

17β-estradiol presented a significant reduction (over 40% (p<0.05) in cellular viral load after 24 hours post-infection. Additionally, estrogen treatment reduces the levels of the TMPRSS2, which are involved with SARS-CoV-2 infectiveness capacity, and hence, reducing the pathogenicity/genesis.
2. Other groups

Compound 145 (artesunate)

Studies to determine the efficacy of anti-viral Compound 145 (artesunate) and Reference Compounds 4 (pyronaridine) and 5 (hydroxychloroquine) in inhibiting the growth in SARS-CoV-2 in human epithelial cells were performed (as referred to in Bae et al., 2020).

Results

The results are summarised in Table 17:

TABLE 17
Results of in vitro experiments

| | | | | Selectivity |
|---|---|---|---|---|
| Compound | Cell Line | IC₅₀ (uM) | CC₅₀ (uM) | Index (SI) |
| Compound 145 | Vero E6 | 53.06 | >100 | >1.885 |
| (artesunate) | Calu-3 | 1.760 | >100 | >56.82 |
| Reference | Vero E6 | 1.084 | 37.09 | 34.22 |
| Compound 4 | Calu-3 | 6.413 | 43.08 | 6.718 |
| (pyronaridine) | | | | |
| Reference | Vero E6 | 1.069 | >100 | >93.55 |
| Compound 5 | Calu-3 | 102.9 | >100 | >0.972 |
| (hydroxychloroquine) | | | | |

The results indicate that in Vero E6 cells, Compound 145 (artesunate) and Reference Compound 4 (pyronaridine) showed comparable inhibitory activity to Reference Compound 5 (hydroxychloroquine). In Calu-3 cells, Compound 145 (artesunate) and Reference Compound 4 (pyronaridine) showed potent inhibitory activity against SARS-COV-2, with Compound 145 demonstrating more potent activity than Reference Compound 4 (pyronaridine). Reference Compound 5 (hydroxychloroquine) showed poor activity in Calu-3 cells. The results of these experiments indicate that anti-viral compounds such as Compound 145 (artesunate) may be effective in the inhibition of SARS-CoV-2 and therefore may be useful in the treatment of COVID-19.

Compound 139 (sulforaphane)

To evaluate the potential virus-inhibitory activity of Compound 139 (sulforaphane), Vero C1008 [Vero 76, clone E6, Vero E6] (ATCC CRL-1586) were exposed in vitro to the drug for 1-2 hours before inoculation with SARS-CoV-2-Wuhan-Hu-1 (as referred to in Ordonez et al., 2021). To evaluate the ability of SFN treatment to reduce viral titers and inflammation in vivo, K18-hACE2 transgenic male mice were inoculated intranasally with 8.4×105 tissue culture infectious dose 50 (TCID50) of SARS-CoV-2/USA/WI1/2020. SFN was administered daily via oral gavage to a subgroup of infected animals starting one day prior to viral inoculation.

Result

The results are summarised in Table 18.

TABLE 18
Results of the study

| | | | virus | cytotoxic |
|---|---|---|---|---|
| | | | selectivity | dose |
| Compound | Cell Line | IC₅₀ (uM) | TI₅₀ (uM) | TD50(uM) |
| Compound 139 | Vero C1008 | 12 | 7 | 73-89 |
| (sulforaphane) | | | | |

In this near-simultaneous drug-infection scenario, Compound 139 (sulforaphane) effectively inhibited both HCoV-OC43 and SARS-CoV-2-Wuhan-Hu-1 virus-associated cell death in non-human primate Vero C1008 cells in a dose-dependent manner revealing comparable median inhibitory concentrations. As a measure of lung injury, the protein concentration in the bronchoalveolar lavage (BAL) was significantly lower in the SFN-treated infected mice compared to untreated infected controls (P<0.0001) suggesting a measure of protective effect of drug pretreatment. The viral burden measured in the alveolar fluid was also significantly lower in treated animals compared to untreated controls, with a 1.15 log reduction in viral titers (P=0.04). Similarly, a 1.5 log reduction in viral lung titers was observed in SFN-treated mice compared to untreated controls, when normalized to PoI2Ra.

Compound 110 (indole-3-carbinol)

the impact of Compound 110 (indole-3-carbinol) on the cytopathic effect (CPE) induced by SARS-CoV-2 infection was evaluated in Vero E6 cells (as referred to in Novelli et al., 2021). the cells were treated with Compound 110 (indole-3-carbinol) using a 3-fold concentration scale ranging between 50 and 0.069 μM. The drug was added at different time points, before (1 h) and after (1, 24, and 48 h) SARS-Cov-2 multiplicity of infection (MOI=0.001). CPE was evaluated 72 h post-infection, when culture media were collected for viral titer measurement (referred to https://doi.org/10.1038/s41419-021-03513-1).

Results

The results showed that Compound 110 (indole-3-carbinol) reduced the SARS-CoV-2-induced CPE in Vero E6 cells at 50 μM by about 60%, when compared to DMSO-treated cells. However, a much greater effect was observed when assessing the impact of Compound 110 (indole-3-carbinol) treatment on the in vitro viral production. It significantly reduced the SARS-CoV-2 production at all the concentrations tested, with a virus yield reduction ranging from 2 to 4 log at the various Compound 110 (indole-3-carbinol) concentrations. Since Compound 110 (Indole-3-carbinol) reduced the viral production not only at 50 μM, when the CPE inhibition is clearly appreciated, but also at 16.67 and 5.56 μM, when SARS-CoV-2-induced CPE was not affected by Compound 110 (indole-3-carbinol), it is likely that 13C reduced the viral release rather than a viral entry and/or replication leading to cell damage. Overall, these data demonstrated that Compound 110 (indole-3-carbinol) exerts a direct anti-SARS-CoV-2 replication activity.

Compound 170 (maraviroc)

Vero cells were pretreated with Compound 170 (maraviroc) and Reference Compound 5 (hydroxychloroquine) before infection for 2 h (as to referred to in Risner et al., 2020). The plate was incubated for 1 hour at 37° C., 5% CO2 to allow the uptake of virus. Cells were incubated at 37° C. and supernatants were collected at indicated times post infection and stored at −80° C. until required for further analyses.

Results

Compound 170 (maraviroc) demonstrated a 30.7-fold decrease (p<0.0001) in viral load as compared to the DMSO control while Reference Compound 5 (hydroxychloroquine) demonstrated a 26.9-fold decrease (p<0.0001).

Compound 2 (17-hydroxyprogesterone-caproate)

Calu-3 and Vero cells were treated with Compound 2 (Hydroxyprogesterone caproate), Reference Compound 6 (remdesivir) and Reference Compound 1 (chloroquine) 24 hours prior to SARS-CoV-2 infection (as referred to in Ko et al., 2020). The infected cells were incubated for another 24 h and then fixed for immunofluorescence. Both viral N protein and host cell nucleus were stained by immunofluorescence and the quantitative analysis to measure the inhibition of virus infection and the cell viability due to drug treatment was conducted.

Result

The results are summarised in Table 19.

TABLE 19
Results of the study

| | IC50 in | IC50 in |
|---|---|---|
| | Vero | Calu-3 |
| Drug name | (μM) | (μM) |
| Reference Compound 6 | 11.41 | 1.3 |
| (remdesivir) | | |
| Compound 2 (17- | 6.3 | 3.87 |
| hydroxyprogesterone | | |
| caproate) | | |
| Reference Compound 1 | 7.28 | 69.2 |
| (chloroquine) | | |

The results showed the in vitro antiviral efficacy of 17-hydroxyprogesterone caproate on Vero and Calu-3 cell lines compared to Reference Compound 6 (remdesivir) and Reference Compound 1 (chloroquine).

Compound 155 (gemcitabine)

The antiviral activity of Compound 155 (gemcitabine) against SARS-CoV2 infection in cell culture was assessed where Reference Compound 1 (chloroquine) was used as a positive control (as referred to in Zhang et al., 2020). Vero-E6 cells infected with SARS-CoV-2 at a multiplicity of infection (MOI) of 0.005 were treated with increased concentrations of the compounds. The viral RNAs in cell culture media were quantified with quantitative real-time RT-PCR.

Results

The results are summarised in Table 19a:

TABLE 19a
Results of the study

| Drug name | EC50 in (μM) | CC50 in (μM) | SI |
|---|---|---|---|
| Compound 155 (gemcitabine) | 1.24 | >40 | 33 |
| Reference Compound 1 | 1.36 | >40 | 31 |
| (chloroquine) | | | |

The results demonstrate that Compound 155 (Gemcitabine) inhibited viral replication in Vero-E6 cells at noncytotoxic concentrations. Additionally, Indirect immunofluorescence assay (IFA) for viral protein expression using anti-NP antibody verified the antiviral activity of the compound. Comparing with DMSO treated group, Gemcitabine treated group showed significantly decreased positive cells with increased concentrations of compound.

Compound 142 (calcitriol)

Vero E6 cells were seeded onto 96-well plates (Corning) at a seeding density of 1×104 cells per 100 μl and incubated overnight (as referred to in Mok et al., 2020). Cells were infected with SARS-CoV-2 infection at a MOI of 1 for 1 h. Final concentrations of 10 μM of Compound 142 (calcitriol) was added to SARS-CoV-2-infected cells. 0.1% DMSO and 100 μM remdesivir were included as vehicle and positive controls, respectively. Cells were then incubated for 4 days at 37° C., 5% CO2 before formalin fixation, analysis and hit selection (referred to https://doi.org/10.1101/2020.06.21.162396).

Results

The result showed that post-infection treatment with 10 μM Compound 142 (calcitriol) resulted in a 1.3 log 10 reduction of SARS-CoV-2 titre in Vero E6 cells.

Compound 192 (resveratrol)

To evaluate the effect of Compound 192 (resveratrol) and compared to Reference Compounds 17 (lopinavir), 18 (ritonavir) and 1 (chloroquine) in vitro, MRCS cells were treated with the compounds diluted in culture media at time of infection by HCoV-229E at multiplicity of infection (MOI)=0.01 (low titer) or 1 (high titer) (as referred to in Pasquereau et al., 2021). The compounds were maintainedwith the virus inoculum during the 2 h incubation period. The inoculum was removed after incubation, and the cells were overlaid with culture media containing diluted compounds. After 48 h of incubation at 37° C., supernatants were collected to quantify viral loads by plaque forming unit (PFU) assay. Vero E6 cells were treated with compounds at time of infection by SARS-CoV-2 (BetaCoV/France, Institut Pasteur). The viral inoculum was removed after incubation and the cells were overlaid with fresh media containing the compound. After 48 h of incubation at 37 C, supernatants were collected to quantify viral loads. Viral RNA was extracted using a guanidinium thiocyanate buffer and magnetic silica beads. Finally, RNA was detected for the SARS-CoV-2 envelop (E) gene using one step qRT-PCR.

Results

EC50, CC50 and selectivity index (SI) for the the drugs tested for human coronavirus (HCoV)-229E replication in MRC5 cells were summarized in Table 19b:

TABLE 19b
Results of the study

| Compound | CC50(uM) | EC50(uM) | SI |
|---|---|---|---|
| Reference Compounds | 102.5 | 8.8163 | 11.62619 |
| 17(lopinavir) and 18 | | | |
| (ritonavir) | | | |
| Reference Compound 1 | 67.9 | 5 | 13.58 |
| (chloroquine) | | | |
| Compound 192 (Resveratrol) | 210 | 4.6 | 45.65217 |

A reduction of the viral titer by 80% with Compound 192 (resveratrol) (50% effective concentration (EC50)=4.6 μM) and Reference Compounds 17 and 18 (lopinavir/ritonavir) (EC50=8.8 μM) and by 60% with Reference Compound 1 (chloroquine; EC50=5 μM) with very limited cytotoxicity. Among these three drugs, Compound 192 (resveratrol) was less cytotoxic (cytotoxic concentration 50 (CC50)=210 μM) than Reference Compounds 17 and 18 (lopinavir/ritonavir) (CC50=102 μM) and Reference Compound 1(chloroquine) (CC50=67 μM). Similarly, among the three drugs with an anti-HCoV-229E activity, only Compound 192 (resveratrol) showed a reduction of the viral titer on SARS-CoV-2 with reduced cytotoxicity.

### Example 5: Human Studies of COVID-19 Treatment

1. Statins

An analysis of 13,981 confirmed COVID-19 cases admitted in 21 hospitals from Hubei Province, China was conducted (as referred to in Zhang et al. 2020). Among them, 1,219 were treated with certain compounds of the invention and reference compounds (treated group) as outlined in Table 20, and the remaining 12,762 had no treatment (untreated group). 861 participants from the treated group were matched at a 1:4 ratio to 3,444 participants from the untreated group.

TABLE 20
Outline of compound administration in the treated group

| | No. of Patients | No. of Patients |
|---|---|---|
| | Administered | Administered |
| Compound | (Matched) | (Unmatched) |
| Compound 8 (atorvastatin) | 1014 | 730 |
| Compound 64 (rosuvastatin) | 190 | 113 |
| Compound 68 (simvastatin) | 22 | 16 |
| Compound 1 (pravastatin) | 16 | 11 |
| Reference Compound 20 | 1 | 0 |
| (fluvastatin) | | |
| Reference Compound 21 | 10 | 7 |
| (pitavastatin) | | |

The tested compounds are compounds from the class of compounds known as “statins”. Among the original participants with COVID-19, participants with incomplete electronic medical records, aged less than 18 or over 85 years, with pregnancy or severe medical conditions, including acute lethal organ injury (i.e., acute coronary syndrome, acute stroke, and severe acute pancreatitis) were excluded. Individuals with pre-existing hypothyroidism (Fellstrom et al., 2009; Truwit et al., 2014) or contraindications for statins use including presented serum levels of CK or aminotransferase of more than five times of the upper limit of normal (ULN) at admission were also excluded (Fellstrom et al., 2009; Truwit et al., 2014). To avoid the confounding effects from non-statin lipid-lowering drugs, participants taking statin combined with other lipid-lowering drugs or those taking non-statin lipid-lowering agents were excluded.

The primary endpoint was defined as 28-day all-cause death. The secondary endpoints were the occurrence of ARDS, septic shock, acute liver injury, acute kidney injury, acute cardiac injury, invasive mechanical ventilation, and intensive care unit admission (data not shown). ARDS and septic shock were defined according to the WHO interim guideline “Clinical management of severe acute respiratory infection when novel coronavirus (2019-nCoV) infection is suspected.” Acute kidney injury was diagnosed by an elevation in serum creatinine level R26.5 mmmol/L within 48 h (Khwaja, 2012). Acute cardiac injury was defined with serum level of cardiac troponin I/T (cTnI/T) above the ULN (Huang et al., 2020; Yancy et al., 2017). Acute liver injury was defined using serum ALT or alkaline phosphatase above 3 folds of ULN (Marrone et al., 2017). The adverse effect of statin was determined by CK to increase above ULN or ALT increase above 3-folds of ULN during follow-up (Truwit et al., 2014).

Results

The results of the study are summarised in Table 21:

TABLE 21
Results of study

| | Unmatched | | Matched | |
|---|---|---|---|---|
| | Incidence | | Incidence | |
| | Rate | | Rate | |
| Patient | (cases/100- | Mortality | (cases/100- | Mortality |
| Group | person-day) | Rate (%) | person-day) | Rate (%) |
| Treated | 0.21 | 5.5 | 0.20 | 5.2 |
| Untreated | 0.27 | 6.8 | 0.37 | 9.4 |

The results indicate that in the unmatched cohort, treated individuals had a lower crude 28-day mortality than untreated individuals (5.5% vs 6.8%). In the matched cohort, the mortality rate for the treated group was markedly lower than in the untreated group (5.2% vs 9.4%). Due to the severe symptoms and comorbidities of subjects in the compound group, the matched untreated group had more severe baseline symptoms and higher proportions of cardiovascular and metabolic comorbidities than the unmatched untreated group. This might account for the increased death rate in the matched untreated group after propensity matching.

The results indicate that treatment using Compound 8 (atorvastatin), Compound 64 (rosuvastatin), Compound 68 (simvastatin) or Compound 1 (pravastatin) may reduce mortality in patients infected with COVID-19.
2. Anti-Hypertensive Drugs

Calcium Channel Blockers (CCBs)

A total of 77 patients were identified from those admitted to a community hospital who tested positive for SARS-CoV-2, who were at or above the age of 65, and who either expired or survived to discharge from hospital between the start of the public health crisis due to the viral disease (earliest admission date of a patient that tested positive at this hospital: Feb. 27, 2020 and Apr. 13, 2020; as referred to in Solaimanzadeh, 2020)). The patients were classified into two groups: (1) Treated with either Compound 51 (nifedipine) or Reference Compound 7 (amlodipine) as part of the CCB group or (2) not treated with either Reference Compound 7 (amlodipine) or Compound 51 (nifedipine) as part of the no-CCB group. Patient outcomes were assessed for survival to discharge or signed out independently against medical advice (AMA) and expiration. Secondary outcomes included the need for intubation and mechanical ventilation. Of these, 18 survived until discharge and 59 expired. One patient signed out against medical advice (AMA) and this was included in the survival group. Seven patients from the expired group were excluded since they died within one day of presentation to hospital and the time frame of clinical deterioration limited potential therapeutic interventions. In order to attempt to age match the case and control groups, five patients were excluded from the expired group since their age was above the eldest patient in the survival group (89 years old). Wth 65 patients left, 41 patients were classified as non-CCB and 24 patients as CCB during hospitalization.

Results

The results are outlined in Table 22:

TABLE 22
Outcomes of the study

| Patient Outcome | CCB | No-CCB |
|---|---|---|
| Survived to Discharge | 12 | 6 |
| Expired | 12 | 35 |
| Number of Patients Intubated and | 1 | 16 |
| Mechanically Ventilated | | |
| Number of Patients that were NOT | 23 | 25 |
| Intubated and Mechanically Ventilated | | |

In patients treated with a CCB, 12 (50%) survived and 12 expired, whereas only six (14.6%) survived and 35 (85.4%) expired in the No-CCB treatment group (p=0.0036). In other word, 67% (12/18) of patients that survived and that were successfully discharged from the hospital were on a CCB, whereas 74% (35/47) of patients that expired were not on a CCB. Patients treated with a CCB were significantly less likely to have undergone intubation and mechanical ventilation. Since only one patient (4.2%) in the CCB group was intubated and mechanically ventilated and 23 (95.8%) were not, whereas 16 (39.0%) were intubated and mechanically ventilated and 25 (61.0%) were not in the No-CCB treatment group (p=0.0026).

The results showed CCBs use was associated with significantly lower case fatality rates (14.6% vs 50%, P<0.01), and lower rates of mechanical ventilation (4.2% vs 39%, P<0.01) on 24 out of 65 COVID-19 patients that were taken CCBs during hospitalization.
3. Anti-psychotic drugs

Study 1 with Compound 34 (fluvoxamine)

In November-December 2020, Covid positive patients in the California Department of Public Health Viral and Rickettsial Disease Laboratory in Richmond, Calif., were offered Compound 34 (fluvoxamine) as an optional therapy (as referred to in Seftel et al., 2021). After evaluation for any specific contraindications or deleterious drug-drug interactions (none excluded), the choice was at the patient's discretion. Compound 34 (fluvoxamine) was prescribed with a 50- to 100-mg loading dose, then 50 mg twice daily for 14 days. AH patients were followed up in-person at 7 and 14 days. No patients were lost to follow up. Of 113 patients, approximately half were asymptomatic when initially tested. The median age was 42 years (interquartile range, 33 to 56), and 75% were men; 84% were Latino, and 14% were white. In total, 65 persons opted for fluvoxamine, and 48 opted for observation alone with no therapy.

Results

The results are summarized in Table 23:

TABLE 23
Results of the study

| | Compound 34 | | | | |
|---|---|---|---|---|---|
| | (fluvoxamine | | No Therapy | | |
| Group | N = 65) | | N = 48 | | P_Value |
| Disease Status at Time of Testing | | | | | 0.064 |
| Asymptomatic | 25 | (38%) | 28 | (58%) | |
| Mild | 24 | (37%) | 9 | (19%) | |
| Moderate | 16 | (25%) | 11 | (23%) | |
| Respiratory Rate | | | | | |
| Day 1 | 17.7 ± 2.9 | | 17.7 ± 3.4 | | 0.95 |
| Day 7 | 12.9 ± 1.6 | | 15.1 ± 4.1 | | 0.001 |
| Hospitalized within 14 | 0 | | 6 | | 0.005 |
| days | | | | | |
| ICU care and/or Death | 0 | | 2 | | — |
| Symptoms at Day 14 d | | | | | <.001 |
| None | 65 | (100%) | 19 | (40%) | |
| 1-3 | 0 | (0%) | 15 | (31%) | |
| 4-6 | 0 | (0%) | 11 | (23%) | |
| ≥7 | 0 | (0%) | 3 | (6%) | |

The incidence of subsequent hospitalization was 0% (0 of 65) with Compound 34 (fluvoxamine) and 12.5% (6 of 48) with observation (P=.005). Two persons required intensive care unit stay with mechanical ventilation, 1 of whom died. Respiratory rates were slightly elevated at diagnosis and improved faster by day 7 with Compound 34 (fluvoxamine) (P=0.001). At day 14, ongoing symptoms were present in 0% (0 of 65) with Compound 34 (fluvoxamine) compared with 60% (29 of 48) with observation alone (P<0.001); 10 (21%) of whom had ≥5 persisting symptoms. The most common persisting symptoms were as follows: persistent anxiety (n=19), difficulty concentrating/memory challenges (n=18), fatigue (n=16), insomnia (n=12), myalgia/arthralgia (n=10), and headache (n=9). No serious adverse events occurred with Compound 34 (fluvoxamine). No adverse events led to early discontinuation. On Dec. 17, 2020, internet-based phase III randomized trial began to confirm these initial results for those with days of COVID-19 symptoms (ClinicalTrials.gov: NCT04668950).

Study 2 with Compound 34 (Fluvoxamine)

A double-blind, randomized, fully remote clinical trial of Compound 34 (fluvoxamine) vs placebo where Participants were community-living, nonhospitalized adults with confirmed SARS-CoV-2 infection, with COVID-19 symptom onset within 7 days and oxygen saturation of 92% or greater was performed(from Apr. 10, 2020, to Aug. 5, 2020; as referred to in ClinicalTrials.gov Identifier: NCT04342663)). 152 participants were enrolled from the St Louis metropolitan area (Missouri and Illinois) and were randomly assigned to receive 100 mg of fluvoxamine (n=80) or placebo (n=72) 3 times daily for 15 days (as outlined in Lenze et al., 2020). Study supplies were delivered to self-quarantined study patients as a package left at their door and the study materials consisted of the study medication, an oxygen saturation monitor, an automated blood pressure monitor, and a thermometer. Participants then self-assessed using the equipment provided and confirmed vital signs within range (systolic blood pressure between 80 mm Hg and 200 mm Hg, diastolic blood pressure between 40 mm Hg and 120 mm Hg, and pulse rate between 50 beats/min and 120 beats/min), pregnancy status when indicated, and oxygen saturation of 92% or greater. AH data collection was done by twice-daily REDCap surveys sent to patients via email, with phone-based data collection as backup to ensure that individuals without Internet access were able to participate. shortness of breath was measured using a continuous scale (0=symptom is not present and 10=symptom is very severe).

Results

The primary and secondary end point results are summarized in Table 24. Overall, the trial showed patients treated with Compound 34 (fluvoxamine) compared with placebo, had a lower likelihood of clinical deterioration over 15 days.

TABLE 24
Results of the study

| Compound 34 | | | | Absolute | | |
|---|---|---|---|---|---|---|
| (fluvoxamine) | | Placebo | | difference | | |
| (n = 80) | | (n = 72) | | (95% CI) | | P value |
| Primary end point | 0 | 6 | (8.3) | 8.7 | (1.8 to 16.4) | 0.009 |
| Clinical deterioration | | | | | | |
| (met both criteria), | | | | | | |
| No. (%) | | | | | | |

Secondary end points (Clinical status on 7-point scale, No. (%))

| | | | | | | |
|---|---|---|---|---|---|---|
| 0 (none) | 80 (100) | 66 | (91.7) | 8.3 | (0.6 to 18.4) | 0.009 |
| Any nonzero value | 0 | 6 | (8.3) | −8.3 | (−18.4 to −0.6) | .009 |
| Clinical status on | 0 | 0.22 | (0.84) | −0.22 | (−0.41 to −0.04) | .02 |
| 7-point scale, mean | | | | | | |
| (SD) | | | | | | |

Clinical deterioration occurred in 0 of 80 patients in the Compound 34 (fluvoxamine) group and in 6 of 72 patients in the placebo group (absolute difference, 8.7% [95% Cl, 1.8%-16.4%] from survival analysis: log-rank P=.009), The fluvoxamine group had 1 serious adverse event and 11 other adverse events, whereas the placebo group had 6 serious adverse events and 12 other adverse events. The fluvoxamine group had 1 serious adverse event and 11 other adverse events, whereas the placebo group had 6 serious adverse events and 12 other adverse events. Pneumonia and gastrointestinal symptoms (such as nausea and vomiting) occurred more often in the placebo group compared with those who received fluvoxamine.
4. Anti-Histamine Drugs

The influence of prior comorbidities and chronic medications use on the risk of COVID-19 in adults were investigated as a population-based cohort study involving 79083 adults aged 50 years in twelve primary care centres in Tarragona (Southern Catalonia, Spain) from 1st March 2020 to 23rd. May 2020 (as referred to in Vila-Corcoles et al., 2020). Risk for suffering COVID-19 was evaluated by Cox regression, estimating multivariable hazard ratios (HRs) adjusted for age, sex, comorbidities and medications use. 2324 cohort members were PCR-tested, with 1944 negative and 380 positive results, which means an incidence of 480.5 PCR confirmed COVID-19 cases per 100000 persons-period. Assessing the total study cohort, the results showed antihistamine (HR 0.47; 95% C1 0.22 to 1.01; p=0.052) were associated with a lower risk.

Compound 16 (Chlorphenamine) and Compound 80 (Desloratadine)

Chlorphenamine maleate nasal spray was tested on a series of four symptomatic COVID-19 patients with mild-moderate risks, and had tested positive for COVID-19 on Sep. 28, 2020 and Oct. 9, 2020 (as referred to in Torres et al., 2021). An elderly white female, with a past medical history of hypertension with unknown treatment and asthma treated with Compound 80 (desloratadine) and Reference Compound 8 (albuterol). Two young adult white and an elderly Hispanic woman with past medical history of chronic rhinitis treated with Reference Compound 9 (cetirizine) or Compound 80 (desloratadine). The patients were non-smokers. All four patients showed rapid improvement of their clinical symptoms with a shorter than average time to negativization on repeat nasopharyngeal swab via RT-PCR. No safety issues were encountered during the course of treatment.
5. RTK inhibitors and inhibitors of RTK pathway

Compound 134 (nintedanib)

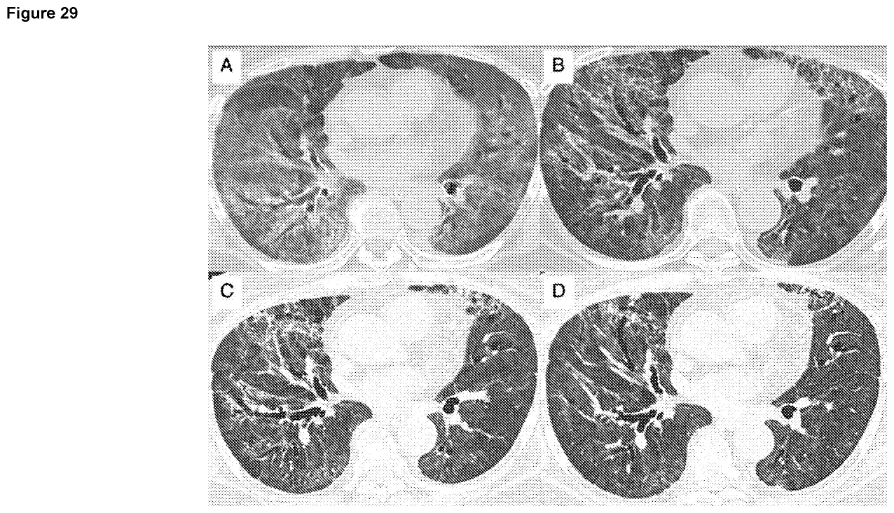

A 78-year-old Japanese woman with no smoking history suffered from COVID-19-induced ARDS (acute respiratory distress syndrome) (FIG. 29-A), which required multidisciplinary management in the intensive care unit, including invasive mechanical ventilation (as referred to in Ogata et al., 2021). She had no underlying disease except for hypertension. The patient was initially treated with the combination therapy of Reference Compound 6 (remdesivir), Reference Compound 10 (dexamethasone) (6 mg/day), and heparin for 10 days, with no beneficial effect on respiratory failure; 14 days after tracheal intubation, her respiratory condition worsened.

Subsequent use of methylprednisolone at a dose of 80 mg/day gradually improved her respiratory condition such that she could be extubated on day 28 after intubation (FIG. 29-B). Regarding the findings from computed tomography of the chest, diffuse ground-glass opacities of the lung were passably attenuated (more than half of the opacities were diminished or improved), whereas diffuse reticular changes with traction bronchiectasis appeared, suggesting the development of lung fibrosis (FIG. 29-C).

On day 12 after transfer, that is, day 65 after the initiation of mechanical ventilation, the patient was started on oral treatment with Compound 134 (nintedanib) at a dose of 300 mg/day. Although the serum level of alanine aminotransferase became elevated as an adverse event, it was safely managed with Compound 134 (nintedanib) dose reduction to 200 mg/day and the use of ursodeoxycholic acid. No other side effects of Compound 134 (nintedanib) were observed.

Results

Pulmonary function tests were performed six weeks after the initiation of Compound 134 (nintedanib) therapy, and the patient demonstrated restrictive ventilatory impairment (vital capacity 1.27 L, vital capacity per predicted 61.7%). After three months of rehabilitation with Compound 134 (nintedanib) and ongoing tapering of prednisolone to 10 mg on alternate day, the respiratory condition and exercise tolerance improved; the patient was able to walk with a walking aid using oxygen at 4 L/min. Ground-glass opacities of the lung were mildly attenuated without further progression of pulmonary fibrosis (FIG. 29-D), while the systemic steroid was tapered to 10 mg every other day of treatment with prednisolone. The patient continues rehabilitation in the hope of hospital discharge to home, alongside the tapering of prednisolone to full withdrawal in a few months.

The results indicate that Compound 134 (nintedanib) may be effective in the treatment of COVID-19-induced pulmonary fibrosis.
6. Corticosteroids and Immunosuppressants

Corticosteroids

A single-center retrospective cohort study (referred to DOI: 10.1128/AAC.01168-20) was performed, which included patients admitted to Hospital Puerta de Hierro-Majadahonda between 4 Mar. 2020 and 7 Apr. 2020 (Fernandez-Cruz et al., 2020). Adult patients diagnosed with COVID-19 pneumonia according to WHO interim guidance and complicated with ARDS and/or an hyperinflammatory syndrome where included. Of them, patients who received corticosteroid therapy according to clinical practice were assigned to the steroid cohort, while patients who did not were assigned to the control cohort. The main outcome variable was in-hospital mortality. The outcomes of patients treated with steroids were compared to those of patients who did not receive steroids.

Results

The results are summarised in Table 25:

TABLE 25
Results of the study

| | | | No. (%) of | | |
|---|---|---|---|---|---|
| | No. (%) of | | non- | | |
| | survivors | | survivors | HR | |
| Steroid exposure | (n 392) | | (n 71) | (95% CI) | P value |
| No corticosteroid | 51 | (76.1) | 16 (23.9) | 0.514 | 0.038 |
| treatment | 341 | (86.1) | 55 (13.9) | (0.274-0.965) | |
| Steroid treatment | | | | | |
| Steroid treatment | | | | 0.360 | 0.035 |
| (adjusted by | | | | (0.139-0.932) | |
| PSM*) | | | | | |

*PSM (propensity score matching)

Steroid treatment reduced mortality by 41.8% relative to the mortality with no steroid treatment (relative risk reduction, 0.42 [95% confidence interval, 0.048 to 0.65])

Compound 32 (Hydrocortisone)

A meta-analysis that pooled data from 7 randomized clinical trials that evacuated the efficacy of corticosteroids in 1703 critically ill patients with COVID-19 (NCT04325061, NCT04327401, NCT04381936 for Reference Compound 10 (dexamethasone); NCT02517489, NCT04348305 NCT02735707 for Compound 32 (hydrocortisone); and NCT04244591 for Reference Compound 11 (methylprednisolone)) was performed (as outlined in WHO REACT working Group, 2020).

The trials were conducted in 12 countries from Feb. 26, 2020, to Jun. 9, 2020, and the date of final follow-up was Jul. 6, 2020. Patients had been randomized to receive systemic Compound 32 (hydrocortisone), Reference Compound 10 (dexamethasone), or Reference Compound 11 (methylprednisolone) (overall 678 patients) or to receive usual care or placebo (1025 patients). The primary outcome measure was all-cause mortality at 28 days after randomization. A secondary outcome was investigator-defined serious adverse events. A total of 1703 patients (median age, 60 years [interquartile range, 52-68 years]: 488 [29%] women) were included in the analysis. Risk of bias was assessed as “low” for 6 of the 7 mortality results and as “some concerns” in 1 trial because of the randomization method. Five trials reported mortality at 28 days, 1 trial at 21 days, and 1 trial at 30 days.

Results

The results of the meta analysis are summarised in Tables 26 and 27.

TABLE 26
Results of the study

| | | | The fixed-effect |
|---|---|---|---|
| | | No | summary odds |
| | Corticosteroids | Corticosteroids | ratio (OR) |
| participants | 678 | 1025 | |
| death | 222 | 425 | 0.66 [95% CI, |
| | | | 0.53-0.82]; P < .001 |

TABLE 27
Results of the study

| | odds ratio (OR) of mortality |
|---|---|
| Reference Compound 10 | 0.64 (95% CI, 0.50-0.82; P < .001) |
| (Dexamethasone)* | |
| Compound 32 | 0.69 (95% CI, 0.43-1.12; P = .13) |
| (hydrocortisone)* | |
| Reference Compound 11 | 0.91 (95% CI, 0.29-2.87; P = .87) |
| (methylprednisolone)* | |

*compared to placebo or usual care.

In this prospective meta-analysis of clinical trials of critically ill patients with COVID-19, administration of systemic corticosteroids, compared with usual care or placebo, was associated with lower 28-day all-cause mortality. The results suggest that Compound 32 (hydrocortisone) may be used as an alternative to Reference Compound 10 (dexamethasone) to treat patients severely ill with COVID-19.

Compound 72 (Triamcinolone)

An interventional study was conducted from June to November 2020 at Datta Meghe Institute of Medical Sciences, Sawangi Meghe, Wardha, Maharashtra, India during the COVID-19 outbreak to check the effect of Compound 72 (triamcinolone) on dysgeusia in COVID positive patients (as referred to in Singh et al., 2021). The 120 enrolled patients were tested at days 1 and 5 after proven infection by RT-PCR test. Administrative information was collected from the hospital database and patients were offered a yes or no questionnaire if they can identify various tastes and smells. History and examination were carried out in person using the questionnaire. The study comprised 120 subjects, among which 60 were in the case group and 60 were in the control group, and the most predominant symptom was cough and breathlessness. AH subjects in the case group and the control croup had anosmia and dysgeusia.

Results

The results for day 5 are summarised in Table 28.

TABLE 28
Results of the study

| | |
|---|---|
| Cases (n = 60) | Controls (n = 60) |

| | YES | NO | YES | NO | p value |
|---|---|---|---|---|---|
| bitter | 53 | 7 | 22 | 38 | <0.05 |
| sweet | 55 | 5 | 14 | 46 | <0.05 |
| salt | 50 | 10 | 13 | 47 | <0.05 |
| sour | 50 | 10 | 13 | 47 | <0.05 |

The results showed Compound 72 (triamcinolone) oral paste improved 83.33% for bitter 91.67% for sweet 83.33% for salty taste 83.34% for sour taste on the fifth day i.e. olfactory and taste function significantly improved in patients with COVID-19. All anosmia and dysgeusia cases who received Compound 72 (triamcinolone) recovered their sense of smell taste within a week.

Compound 17 (Ciclosporin)

The data of 607 patients, with median age 69 years and 35% female were collected at Hospital Universitario Quironsalud Madrid, sited in Pozuelo de Alarcon, Madrid, Spain (referred to in Guisado-Vasco et al., 2020). At the end of the study period, 194 (31-92%) remained in the hospital and were followed until 12 May 2020. During the in-hospital follow-up, 466 patients survived (76-08%) and 141 died (23-2%). The primary end-point was in-hospital mortality. The secondary outcomes were the total length of stay, the number of patients admitted to the intensive care unit (ICU), length of stay in the ICU, percentage who required mechanical ventilation, or non-invasive ventilation.

Result

Table 29 shows the in-hospital mortality relative to Compound 17 (ciclosporin).

TABLE 29
Results of the study

| Treated with | | | | | | | |
|---|---|---|---|---|---|---|---|
| Compound 17 | All patients | | Mortality | | | | P value |
| (ciclosporin)? | (n, % all) | | (n, % treated) | | IC 95% | | (χ² test) |
| Yes | 253 | 41.68% | 36 | 14.23% | 10.46% | 19.07% | <0.001 |
| No | 354 | 58.32% | 105 | 29.66% | 25.14% | 34.62% | |

The results showed the positive effect of Compound 17 (ciclosporin) on in-hospital mortality associated with severe COVID-19, used as a first-line drug. Treatment with Compound 17 (ciclosporin) showed a reduction in the odds ratio for death in hospitalized patients affected by severe COVID-19 (odds ratio (0.24, [0.12-0.46]); p<0.001.
7. Bruton's Tyrosine Kinase (BTK) Inhibitors

Compound 133 (ibrutinib)

6 identified patients receiving ibrutinib for WM (Waldenstrom macroglobulinemia) who were diagnosed with COVID-19 were considered to assess the effect of Compound 133 (ibrutinib) on COVID-19 disease (as referred to in Treon et al., 2020). Their median age was 66 years, and 5 were on the recommended treatment dose of 420 mg/d; the sixth patient was on a reduced dose of 140 mg/d because of arthralgias. For all patients, the median time on ibrutinib was 52 months. Their median time with COVID-19—related symptoms prior to diagnostic testing was 5 days, and the median time since diagnosis of COVID-19 was 22 days.

Results

All 6 patients experienced cough and fever as prodromal symptoms. The 5 patients treated with Compound 133 (ibrutinib), 420 mg/d, did not experience dyspnea and did not require hospitalization. Their course was marked by steady improvement, and resolution or near resolution of COVID-19—related symptoms during the follow-up period. The clinical characteristics of patients with WM on ibrutinib with COVID-19 infection are summarised in Table 30.

TABLE 30
Results of the study

| Demographics | Patient 1 | Patient 2 | Patient 3 | Patient 4 | Patient 5 | Patient 6 |
|---|---|---|---|---|---|---|
| Sex | M | M | F | F | M | M |
| Time since B-cell diagnosis, | 39 | 54 | 95 | 202 | 52 | 107 |
| mo | | | | | | |
| Received treatment prior | No | No | Yes | Yes | No | Yes |
| to ibrutinib for WM | | | | | | |
| Time on ibrutinib, mo | 39 | 54 | 83 | 50 | 47 | 85 |
| Dose of ibrutinib, mg/d | 420 | 420 | 420 | 420 | 420 | 140- |
| | | | | | | HELD-420 |

COVID-19 symptoms

| | | | | | | |
|---|---|---|---|---|---|---|
| Time with symptoms prior | 5 | 2 | 6 | 7 | 10 | 5 |
| to COVID-19 diagnostic | | | | | | |
| testing, d | | | | | | |
| Time since COVID-19 | 24 | 20 | 17 | 28 | 13 | 29 |
| diagnostic testing, d | | | | | | |
| Cough | Yes | Yes | Yes | Yes | Yes | Yes |
| Fever | Yes | Yes | Yes | Yes | Yes | Yes |
| Dyspnea | No | No | No | No | No | Yes |
| Sore throat | Yes | No | No | No | No | Yes |
| Taste loss | No | No | Yes | No | Yes | No |
| Smell loss | No | No | Yes | No | Yes | No |
| Hospitalization | No | No | No | No | No | Yes |
| Required ICU admission | No | No | No | No | No | Yes |
| Required supplemental O2 | No | No | No | No | No | Yes |
| Required mechanical | No | No | No | No | No | Yes |
| ventilation | | | | | | |
| Other COVID-19 symptoms | No | Anorexia | Diarrhea | Headache | No | No |
| Other medication for | HCQ, AZ | NA | No | NA | No | HCQ, AZ, |
| COVID-19 | | | | | | TOCI |

Disposition

| | | | | | | |
|---|---|---|---|---|---|---|
| COVID-19 symptoms resolved | No | Yes | Yes | Yes | Yes | No |
| COVID-19 symptoms persist | Yes | No | Yes | Yes | No | Yes |
| COVID-19 symptoms improved | Yes | Yes | Yes | Yes | Yes | Yes |

Compound 35 (Fostamatinib)

Rigel Pharmaceuticals has reported that the Phase II clinical trial of Compound 35 (fostamatinib) in treating hospitalised Covid-19 patients met the primary endpoint of safety (https://www.clinicaltrialsarena.com/news/rigel-fostamatinib-primary-endpoin/). The study enrolled 59 subjects who needed supplemental oxygen via nasal canula or non-invasive ventilation, mechanical ventilation, or extracorporeal membrane oxygenation. They were randomised into a 1:1 ratio to receive an oral twice-daily dose of Compound 35 (fostamatinib) plus standard of care (SOC) or matching placebo plus SOC for 14 days. They were also followed for 60 days.

Results

Compound 35 (fostamatinib) halved the chances of occurrence of serious adverse events. No deaths were reported in the Compound 35 (fostamatinib) cohort versus three deaths observed in the placebo arm. The trial had a total of four intubated patients on mechanical ventilation. Data showed that the two patients receiving Compound 35 (fostamatinib) improved in seven days and were off the ventilator, while two others in the placebo group succumbed to death. Compound 35 (fostamatinib) was superior to placebo in expediting improvement in clinical status by day 15 and 29. The number of days in the ICU was three days in the Compound 35 (fostamatinib) group versus seven in the placebo group.
8. Estrogen Receptor Agonist

The effect of Compound 25 (estradiol) on COVID-19 patients was assessed (as referred to in Seeland et al., 2020). The primary outcome for Compound 25 (estradiol) therapy was death. A logistic regression analysis was performed for the combined outcome variable “death.” 16,891 peri- and post-menopausal women (50+) were included, either with estradiol hormone usage (“user”) or without regular usage of estradiol hormone (“non-user”). It was required that patients must have taken (or not taken) the drug within the last 1 year.

Results

After matching the data of propensity scores for women aged 50+ with COVID-19, there was found to be a benefit for the Compound 25 (estradiol) hormone-user group vs. the non-user group, as regards to fatality. The OR calculated via logistic regression analysis for the combined outcome variable was 0.33 [0.18, 0.62] and the hazard ratio (HR) was 0.29 [0.11, 0.76] for the estradiol non-user vs. hormone-user group (Table 31).

TABLE 31
Results of the study

| Compound 25 | | | |
|---|---|---|---|
| (estradiol) | Patients in | Odds ratio | Hazard ratio |
| usage cohort | cohort | [95% CI] | [95% CI] |
| User | 439 | 0.33 [0.18, 0.62] | 0.29 [0.11, 076] |
| Non-user | 16278 | | |

The average age across both groups was 64.2 years. The risk reduction for fatality from 6.6% (non-user) to 2.3% (user) was statistically significant (p<0.0001). Survival probabilities were calculated via Kaplan-Meier analyses, which revealed a significant difference (p<0.0001) among women for the 50+ Compound 25 (estradiol) user group (n=439) compared to the age-matched Compound 25 (estradiol) non-user group (n=16,278). Considering the survival probability at 180 days after diagnosis of SARS-CoV-2 infection (index event), n=10 women died in the estradiol user group and n=1072 died in the non-user group. Peri- and post-menopausal women, aged 50+, benefitted from Compound 25 (estradiol) hormone use: the 180-day survival probability for this cohort was 96.7%, compared to 84.9% for the non-user group.
9. Other Groups

Compound 137 (etoposide)

Thirteen patients received Compound 137 (etoposide) (50-150 mg/m²) out of 709 COVID-19 patients admitted to the center during the study period (March 2 to Apr. 10, 2020; as referred to in Montero-Baladia et al., 2020). Overall, 412/709 developed ARDS (58.1%), of which 169 received Reference Compound 11 (methylprednisolone) and Reference Compound 12 (tocilizumab) (23.8%), and 13 patients received Compound 137 (etoposide). Two out of 13 patients were excluded because they were already intubated. A total of 11 patients (1.8%), 9 males and 2 females, with a median age of 58 (range, 41 to 79) were included.

Results

Median PaO₂/FiO₂ at admission was 98 (range, 52 to 174). Following Compound 137 (etoposide) treatment, the PaO₂/FiO₂ ratio improved an average of 195%. Three patients needed mechanical ventilation. Nine patients fully recovered and were finally discharged home. Two patients died because of thrombotic complications. Noticeably, only 1-2 doses of Compound 137 (etoposide) were enough to observe clinical improvement among severely ill COVID-19 patients. these preliminary results on 11 patients confirmed the safety and efficacy of Compound 137 (etoposide) as adjunctive salvage treatment for critically ill COVID-19 ARDS patients, exhibiting systemic hyperinflammation and previously treated with corticosteroids and interleukin inhibitors.

Compound 21 (Curcumin)

A randomized, double-blind, placebo-controlled study in which a total of 40 COVID-19 patients (aged 19-69 years) and 40 healthy controls were included, who were referred to Imam Reza Hospital of Tabriz University of Medical Sciences (as referred to in Valizadeh et al., 2020). The patient group was also divided into the groups of Compound 21 (nano-curcumin) (n=20) and placebo (n=20), based on the intervention method. The treatment group received 160 mg of Compound 21 (nano-curcumin) in four 40 mg capsules daily for 14 days and the control group received the placebo capsule. Additionally, all COVID-19 subjects in both Compound 21 (nano-curcumin) and placebo groups, received Reference Compound 12 (betaferon) 300 μg subcutaneously every other day until 5 days, Reference Compound 13 (bromhexine) 8 mg tablets every 8 h, and Compound 8 (atorvastatin). Peripheral blood samples of all subject were collected twice; first, prior to the intervention, 8 ml of fasting blood samples were collected in EDTA tubes and the second sampling was conducted at the end of the study in the same situation.

Results

mRNA expression level measured by qPCR is summarized in Table 32.

TABLE 32
Results of the study

| | | | | Compound 21 | | | | | | |
|---|---|---|---|---|---|---|---|---|---|---|
| | | | | (curcumin group) | | | placebo group | | | |
| | patients | Healthy | p-value | before | after | p-value | before | after | p-value | p-value |
| IL1β | 2.9 | 0.98 | <0.0001 | 1.01 | 0.56 | 0.0017 | 1.01 | 1.16 | NS | 0.0001 |
| IL6 | 0.99 | 4.19 | <0.0001 | 0.98 | 0.58 | 0.0003 | 0.97 | 1.15 | NS | <0.0001 |

The results demonstrate that that the expression level of IL-1βdecreased after treatment with Compound 21 (nano-curcumin) (P=0.0017) compared with pre-treatment. Additionally, a significant reduction was observed in the Compound 21 (nano-curcumin) group compared to the placebo group (P=0.0001). Downregulation of the IL-6 mRNA expression was also observed in the Compound 21 (nano-curcumin group) after treatment (P=0.0003) and compared with the placebo group (P=0.0001). There were no statistically significant differences in the expression level of IL-18 and TNF-α before and after intervention in the Compound 21 (nano-curcumin) group as well as the placebo groups.

Compound 142 (Calcitriol)

76 consecutive patients hospitalized with COVID-19 infection, clinical picture of acute respiratory infection, confirmed by a radiographic pattern of viral pneumonia and by a positive SARS-CoV-2 PCR with CURB65 severity scale (recommending hospital admission in case of total score >1) were collected to investigate the effect of calcifediol (precursor for Compound 142 (calcitriol)) treatment among patients hospitalized for COVID-19 (as outlined in Castillo et al., 2020). All hospitalized patients received as best available therapy the same standard care, (per hospital protocol), of a combination of Reference Compound 5 (hydroxychloroquine) (400 mg every 12 h on the first day, and 200 mg every 12 h for the following 5 days), and Reference Compound 19 (azithromycin; 500 mg orally for 5 days). Eligible patients were allocated at a 2 calcifediol:1 no calcifediol ratio through electronic randomization on the day of admission to take oral calcifediol (0.532 mg), or not. Patients in the calcifediol treatment group continued with oral calcifediol (0.266 mg) on day 3 and 7, and then weekly until discharge or ICU admission. Outcomes of effectiveness included rate of ICU admission and deaths.

Results

Requirements for admission to the Intensive Care Unit, in patients hospitalized with COVID-19 (treated or not with calcifediol).

| | Without | With | |
|---|---|---|---|
| | Calcifediol | Calcifediol | p value |
| Patient | Treatment | Treatment | (Fischer |
| Outcomes | (n = 26) | (n = 50) | test) |
| Need for ICU | — | — | <0.001 |
| Not requiring | 13 (50) | 49 (98) | — |
| ICU, n (%) | | | |
| Requiring | 13 (50) | 1 (2) | — |
| ICU, n (%) | | | |

The result demonstrated that administration of a high dose of the compound significantly reduced the need for ICU treatment of patients requiring hospitalization due to proven COVID-19.

### Example 6: Ongoing Clinical Trials

A list of compounds of the invention currently undergoing clinical trials is presented in Table 33:

TABLE 33
Compounds of the invention undergoing clinical trials

| Compound ID | Clinical Trial ID |
|---|---|
| Compound 145 | PACTR202006899597082, |
| (artesunate) | NCT04532931, NCT04475107, |
| | NCT04701606, NCT04695197, |
| | NCT04387240, NCT04374019 |
| Compound 8 | IRCT20200413047062N1, |
| (atorvastatin) | PACTR202007720062393, |
| | IRCT20190727044343N2, |
| | IRCT20190831044653N5, |
| | IRCT20200408046990N3, |
| | IRCT20200906048638N1, |
| | IRCT20201028049175N3, |
| | NCT04333407, NCT04380402, |
| | NCT04801940, NCT04631536, |
| | NCT04466241, NCT04486508 |
| Compound 9 | NCT04456153, NCT04339426 |
| (atovaquone) | |
| Compound 17 | EUCTR2020-001262-11-ES, |
| (Ciclosporin) | EUCTR2020-001437-12-ES, |
| | EUCTR2020-002123-11-ES, |
| | EUCTR2020-003505-58-IT, |
| | NCT04412785, NCT04540926, |
| | NCT04392531, NCT04492891, |
| | NCT04451239, |
| | IRCT20200426047206N3 |
| Compound 21 | CTRI/2021/02/031520, |
| (curcumin) | EUCTR2020-001303-16-FR, |
| | IRCT20080901001165N56, |
| | IRCT20121216011763N46, |
| | IRCT20170128032241N3, |
| | IRCT20200418047119N1, |
| | IRCT20200514047445N1, |
| | IRCT20200611047735N1 |
| Compound 22 | EUCTR2020-003505-58-IT, |
| (cyclosporin-A) | NCT04451239 |
| Compound 183 | NCT04676867 |
| (dalcetrapib) | |
| Compound 113 | NCT04830735 |
| (dasatinib) | |
| Compound 200 | NCT04482621 |
| (decitabine) | |
| Compound 190 | EUCTR2020-002027-10-SE, |
| (enzalutamide) | NCT04475601, NCT04456049 |
| Compound 83 | EUCTR2015-002340-14-NL |
| (erythromycin) | |
| Compound 25 | CTRI/2020/09/027622, |
| (estradiol) | NCT04359329 |
| Compound 137 | NCT04356690 |
| (etoposide) | |
| Compound 30 | NCT04330300 |
| (felodipine) | |
| Compound 34 | EUCTR2020-002299-11-HU, |
| (fluvoxamine) | IRCT20131115015405N4, |
| | NCT04718480, NCT04342663, |
| | NCT04711863, NCT04727424, |
| | NCT04668950 |
| Compound 35 | EUCTR2020-001750-22-GB, |
| (fostamatinib) | NCT04581954, NCT04629703, |
| | NCT04579393 |
| Compound 32 | CTRI/2020/09/027615, |
| (hydrocortisone) | EUCTR2015-002340-14-NL, |
| | EUCTR2020-001395-15-DK, |
| | IRCT20120215009014N354, |
| | NCT04359511, NCT04456439, |
| | NCT04348305, NCT02735707 |
| Compound 133 | NCT04375397, NCT04665115, |
| (ibrutinib) | NCT04439006 |
| Compound 79 | NCT04429555 |
| (ibudilast) | |
| Compound 174 | EUCTR2020-001243-15-BE, |
| (itraconazole) | NCT04577378 |
| Compound 39 | NCT04361643 |
| (lenalidomide) | |
| Compound 195 | ACTRN12620000731998, |
| (losartan) | CTRI/2020/05/025319, |
| | PACTR202006473370201, |
| | EUCTR2020-001766-11-FR, |
| | IRCT20180802040678N4, |
| | ISRCTN48734830, NCT04335123, |
| | NCT04428268, NCT04643691, |
| | NCT04343001, NCT04328012, |
| | NCT04606563, NCT04349410, |
| | NCT04447235, NCT04312009, |
| | NCT04311177, NCT04340557, |
| | NCT04330300 |
| Compound 170 | NCT04441385, NCT04475991, |
| (maraviroc) | NCT04435522, NCT04710199 |
| Compound 96 | IRCT20200418047122N1 |
| (niacin) | |
| Compound 179 | NCT04330300 |
| (nicardipine) | |
| Compound 51 | ChiCTR2000032314, NCT04330300 |
| (nifedipine) | |
| Compound 180 | NCT04330300 |
| (nimodipine) | |
| Compound 134 | JPRN-jRCTs051200036, |
| (nintedanib) | ChiCTR2000031453, |
| | EUCTR2020-002114-40-FR, |
| | NCT04541680, NCT04338802, |
| | NCT04619680 |
| Compound 52 | NCT04330300 |
| (nitrendipine) | |
| Compound 58 | CTRI/2020/09/027615, |
| (prednisone) | EUCTR2020-001553-48-FR, |
| | EUCTR2020-001622-64-ES, |
| | RPCEC00000322, NCT04359511, |
| | NCT04451174, NCT04534478, |
| | NCT04795583, NCT04551781, |
| | NCT04492358, NCT04344288, |
| | NCT04782700 |
| Compound 60 | NCT04366739, NCT04354805 |
| (promazine) | |
| Compound 167 | NCT04553705, NCT04338698 |
| (quinine) | |
| Compound 56 | EUCTR2020-003936-25-IT |
| (raloxifene) | |
| Compound 192 | CTRI/2020/06/026256, |
| (resveratrol) | CTRI/2020/07/026514, |
| | CTRI/2020/07/026515, |
| | IRCT20080901001165N56, |
| | IRCT20181030041504N1, |
| | IRCT20200112046089N1, |
| | NCT04400890, NCT04799743, |
| | NCT04542993 |
| Compound 63 | EUCTR2020-002282-33-DE, |
| (rivaroxaban) | EUCTR2020-001302-30-AT, |
| | NCT04333407, NCT04757857, |
| | NCT04715295, NCT04504032, |
| | NCT04416048, NCT04351724, |
| | NCT04508023, NCT04640181, |
| | NCT04662684, NCT04736901, |
| | NCT04394377, NCT04324463, |
| | NCT04508439 |
| Compound 64 | EUCTR2020-001319-26-ES, |
| (rosuvastatin) | NCT04359095, NCT04472611 |
| Compound 68 | ISRCTN67000769, |
| (simvastatin) | PACTR202006473370201, |
| | NCT02735707, NCT04343001, |
| | NCT04348695 |
| Compound 139 | EUCTR2020-003486-19-GB |
| (sulforaphane) | |
| Compound 70 | NCT04568096, NCT04389580 |
| (tamoxifen) | |
| Compound 69 | NCT02735707 |
| (ticagrelor) | |
| Compound 157 | IRCT20190624043993N3, |
| (tretinoin) | NCT04577378, NCT04389580, |
| | NCT04361422, NCT04353180, |
| | NCT04396067, NCT04663906, |
| | NCT04382950, NCT04730895 |
| Compound 106 | ACTRN12621000309886, |
| (trichostatin-A) | IRCT20210111050011N1, |
| | ISRCTN77689525, |
| | JPRN-jRCT2031200092, |
| | NCT04645563 |
| Compound 73 | IRCT20200329046892N1 |
| (trifluoperazine) | |
| Compound 77 | NCT04513314 |
| (valproic-acid) | |
| Compound 71 | DRKS00021732, |
| (valsartan) | EUCTR2020-001320-34-NL, |
| | EUCTR2020-001431-27-DE, |
| | NCT04335786, NCT04330300 |
| Compound 169 | EUCTR2020-001951-42-PL, |
| (verapamil) | NCT04351763, NCT04330300 |

The interest in said compounds demonstrates the utility of a predictive phenotypic drug discovery approach which can be used to identify compounds which would not typically be selected by a target-driven drug discovery approach.

### REFERENCES

Cui et al., Origin and evolution of pathogenic coronaviruses, Nat. Rev. Microbiology., 2019, 17, 181-192.
Singhal, T., A Review of Coronavirus Disease-2019 (COVID-19), Ind. J. Pediatrics, 2020, 87(4), 281-286.
Corman et al. Hosts and Sources of Endemic Human Coronaviruses, Advances in Virus Research. 100, 163-88.
Cao, X. COVID-19: immunopathology and its implications for therapy, Nat. Rev. Immunology, https://doi.org/10.1038/s41577-020-0308-3.
Mehta et al., COVD-19: consider cytokine storm syndromes and immunosuppression, The Lancet, 2020, 395, 10229, 1033-1034.
Sanders et al. Pharmacologic Treatments for Coronavirus Disease 2019 (COVID-19) https://doi:10.1001/jama.2020.6019
Kaur S. and Singh S. Biofilm formation by Aspergillus fumigatus. Med. Mycol., 2014, 52, 2-9.
Pasqualotto A. C., Powell G., Niven R. and Denning D. W. The effects of antifungal therapy on severe asthma with fungal sensitization and allergic bronchopulmonary aspergillosis. Respirology, 2009, 14, 1121-127.
Chishimba L., Niven R. M., Fom M., Cooley J. and Denning D. W. Voriconazole and Posaconazole Improve Asthma Severity in Allergic Bronchopulmonary Aspergillosis and Severe Asthma with Fungal Sensitization. Pharmacotherapy, 2012, 49, 423-433.
Bafadhel M., McKenna S., Aqbetile J., Fairs A., Desai D., Mistry V., Morley J. P., Pancholi M., Pavord I. D., Wardlaw A. J., Pashley C. H. and Brightling C. E. Aspergillus fumigatus during stable state and exacerbations of COPD. Eur. Respir. J., 2014, 43, 64-71.
Chotirmall S. H., O'Donoghue E., Bennett K., Gunaratnam C., O'Neill S. J. and McElvaney N. G. Sputum Candida albicans presages FEV₁ decline and hospital-treated exacerbations in cystic fibrosis. Chest, 2010, 138, 1186-95.
Agbetile, J., Fairs, A., Desai, D., Hargadon, B., Bourne, M., Mutalithas, K., Edwards, R., Morley, J. P., Monteiro, W. R., Kulkarni, N. S., Green, R H, Pavord, I. D., Bradding, P., Brightling, C. E., Wardlaw, A. J. and Pashley, C. H. Isolation of filamentous fungi from sputum in asthma is associated with reduced post-bronchodilator FEV1. Clin. Exp. Allergy, 2012, 42, 782-91.
King T E Jr, et al., Lancet, 2011, 3; 378(9807):1949-61.
Selman M, et al., Ann Intern Med., 2001, 16; 134(2):136-51.
Du Bois R M., Nat Rev Drug Discov., 2010, 9(2):129-40
Castriotta R J, et al., Chest, 2010, 138(3):693-703
Bringardner et al. Antioxid. Redox Signal, 2008, 10(2), 287-301.
Straus, et al., FDA approved calcium channel blockers inhibit SARS-CoV-2 infectivity in epithelial lung cells bioRxiv 2020.07.21.214577.
Weston et al., Broad Anti-coronavirus Activity of Food and Drug Administration-Approved Drugs against SARS-CoV-2 In Vitro and SARS-CoV In Vivo, Journal of Virology October 2020, 94 (21) e01218-20
Hoagland et al., Modulating the transcriptional landscape of SARS-CoV-2 as an effective method for developing antiviral compounds, bioRxiv 2020.07.12.199687
Hou et al., Testing of the inhibitory effects of loratadine and desloratadine on SARS-CoV-2 spike pseudotyped virus viropexis, Chemico-Biological Interactions, 338, 2021, 109420.
Westover et al., In Vitro Virucidal Effect of Intranasally Delivered Chlorpheniramine Maleate Compound Against Severe Acute Respiratory Syndrome Coronavirus 2. Cureus. 2020 Sep. 17; 12(9):e10501.
Yang et al., Identification of SARS-CoV-2 entry inhibitors among already approved drugs. Acta Pharmacol Sin (2020). https://doi.org/10.1038/s41401-020-00556-6
Gassen et al., Analysis of SARS-CoV-2-controlled autophagy reveals spermidine, MK-2206, and niclosamide as putative antiviral therapeutics, bioRxiv 2020.04.15.997254.
Lemes et al., 1713-estradiol reduces SARS-CoV-2 infection in vitro. Physiol Rep. 2021; 9:e14707.
Bae et al., Pyronaridine and artesunate are potential antiviral drugs against COVID-19 and influenza, bioRxiv 2020.07.28.225102
Ordonez et al., Sulforaphane exhibits in vitro and in vivo antiviral activity against pandemic SARS-CoV-2 and seasonal HCoV-OC43 coronaviruses, bioRxiv 2021.03.25.437060
Novelli et al., Inhibition of HECT E3 ligases as potential therapy for COVID-19. Cell Death Dis 12, 310 (2021).
Risner et al., Maraviroc inhibits SARS-CoV-2 multiplication and s-protein mediated cell fusion in cell culture, bioRxiv 2020.08.12.246389
Ko et al., Comparative analysis of antiviral efficacy of FDA-approved drugs against SARS-CoV-2 in human lung cells: Nafamostat is the most potent antiviral drug candidate, bioRxiv 2020.05.12.090035
Zhang et al., In-Hospital Use of Statins Is Associated with a Reduced Risk of Mortality among Individuals with COVID-19. Cell Metab. 2020 Aug. 4; 32(2):176-187.e4. doi: 10.1016/j.cmet.2020.06.015.
FellstrÖm et al., Rosuvastatin and cardiovascular events in patients undergoing hemodialysis. N. Engl. J. Med. 2009; 360:1395-1407.
Truwit et al., Rosuvastatin for sepsis-associated acute respiratory distress syndrome. N. Engl. J. Med. 2014; 370:2191-2200.
Khwaja A. KDIGO clinical practice guidelines for acute kidney injury. Nephron Clin. Pract. 2012; 120:c179-c184.
Huang et al., Clinical features of patients infected with 2019 novel coronavirus in Wuhan, China. Lancet. 2020; 395:497-506.
Yancy et al., 2017 ACC/AHA/HFSA focused update of the 2013 ACCF/AHA Guideline for the Management of Heart Failure: a report of the American College of Cardiology/American Heart Association Task Force on Clinical Practice Guidelines and the Heart Failure Society of America. Circulation. 2017; 136:e137-e161.
Marrone et al., Drug-induced liver injury 2017: the diagnosis is not easy but always to keep in mind. Eur. Rev. Med. Pharmacol. Sci. 2017; 21(Suppl):122-134.
Solaimanzadeh I (May 12, 2020) Nifedipine and Amlodipine Are Associated With Improved Mortality and Decreased Risk for Intubation and Mechanical Ventilation in Elderly Patients Hospitalized for COVID-19. Cureus 12(5): e8069. doi:10.7759/cureus.8069
Seftel et al., Prospective Cohort of Fluvoxamine for Early Treatment of Coronavirus Disease 19, Open Forum Infectious Diseases, Volume 8, Issue 2, February 2021, ofab050.
Torres et al., Chlorpheniramine maleate nasal spray in COVID-19 patients: Case Series, 15 Jan. 2021, PREPRINT (Version 1) available at Research Square [https://doi.org/10.21203/rs.3.rs-138252/v1]
Vila-Córcoles et al., Influence of prior comorbidities and chronic medications use on the risk of COVID-19 in adults: a population-based cohort study in Tarragona, Spain, BMJ Open 2020; 10:e041577. doi: 10.1136/bmjopen-2020-041577
Ogata et al., Nintedanib treatment for pulmonary fibrosis after coronavirus disease 2019. Respirology Case Reports, 9(5), e00744. https://doi.org/10.1002/rcr2.744.
Fernández-Cruz, et al., A Retrospective Controlled Cohort Study of the Impact of Glucocorticoid Treatment in SARS-CoV-2 Infection Mortality, 2020, 64 (9) e01168-20; DOI: 10.1128/AAC.01168-20.
The WHO Rapid Evidence Appraisal for COVID-19 Therapies (REACT) Working Group. Association Between Administration of Systemic Corticosteroids and Mortality Among Critically III Patients With COVID-19: A Meta-analysis. JAMA. 2020; 324(13):1330-1341. doi:10.1001/jama.2020.17023.
Veer Singh et al., The outcome of fluticasone nasal spray on anosmia and triamcinolone oral paste in dysgeusia in COVID-19 patients, American Journal of Otolaryngology, Volume 42, Issue 3, 2021, 102892
P. Guisado-Vasco et al. Clinical characteristics and outcomes among hospitalized adults with severe COVID-19 admitted to a tertiary medical center and receiving antiviral, antimalarials, glucocorticoids, or immunomodulation with tocilizumab or cyclosporine: A retrospective observational study (COQUIMA cohort) EClinicalMedicine 28 (2020) 100591.
Treon et al., The BTK inhibitor ibrutinib may protect against pulmonary injury in COVID-19— infected patients. Blood 2020; 135 (21): 1912-1915.
Seeland et al., Evidence for treatment with estradiol for women with SARS-CoV-2 infection. BMC Med 18, 369 (2020).
Montero-Baladia et al. Etoposide treatment adjunctive to immunosuppressants for critically ill COVID-19 patients. The Journal of infection vol. 81,3 (2020): 452-482.
Valizadeh et al., Nano-curcumin therapy, a promising method in modulating inflammatory cytokines in COVID-19 patients, International Immunopharmacology, Volume 89, Part B, 2020, 107088.
Lenze et al., Fluvoxamine vs Placebo and Clinical Deterioration in Outpatients With Symptomatic COVID-19, JAMA. 2020; 324(22):2292-2300.
Raymonda et al. Pharmacologic profiling reveals lapatinib as a novel antiviral against SARS-CoV-2 in vitro bioRxiv 2020.11.25.398859; doi: https://doi.org/10.1101/2020.11.25.398859
Zhang et al., Gemcitabine, lycorine and oxysophoridine inhibit novel coronavirus (SARS-CoV-2) in cell culture, Emerging Microbes & Infections, 9:1, 1170-1173.
Mok et al., Calcitriol, the active form of vitamin D, is a promising candidate for COVID-19 prophylaxis, bioRxiv 2020.06.21.162396; doi: https://doi.org/10.1101/2020.06.21.162396
Pasquereau, S et al., Resveratrol Inhibits HCoV-229E and SARS-CoV-2 Coronavirus Replication In Vitro. Viruses 2021, 13, 354.
Castillo et al., Effect of calcifediol treatment and best available therapy versus best available therapy on intensive care unit admission and mortality among patients hospitalized for COVID-19: A pilot randomized clinical study. J Steroid Biochem Mol Biol. 2020; 203:105751.

1. A compound selected from Compound 30 (felodipine) and Compounds 1 to 29 and 31 to 203 and prodrugs thereof and pharmaceutically acceptable salts and solvates thereof for use in the treatment or prevention of a disease selected from the group consisting of a positive-sense single-stranded RNA virus ((+)ssRNA virus) infection, a disease associated with a positive-sense single-stranded RNA virus ((+)ssRNA virus) infection and lung inflammation.

2. A method of treatment or prevention of a coronavirus infection of a disease selected from the group consisting of a positive-sense single-stranded RNA virus ((+)ssRNA virus) infection, a disease associated with a positive-sense single-stranded RNA virus ((+)ssRNA virus) infection and lung inflammation in a subject by administering to said subject an effective amount of a compound selected from Compound 30 (felodipine) and Compounds 1 to 29 and 31 to 203 and prodrugs thereof and pharmaceutically acceptable salts and solvates thereof.

3. Use of a compound selected from Compound 30 (felodipine) and Compounds 1 to 29 and 31 to 203 and prodrugs thereof and pharmaceutically acceptable salts and solvates thereof in the manufacture of a medicament for the treatment or prevention of a disease selected from the group consisting of a positive-sense single-stranded RNA virus ((+)ssRNA virus) infection, a disease associated with a positive-sense single-stranded RNA virus ((+)ssRNA virus) infection and lung inflammation.

4. The compound for use, method or use according to any one of claims 1 to 3, wherein the disease is a positive-sense single-stranded RNA virus ((+)ssRNA virus) infection.

5. The compound for use, method or use according to claim 4, wherein the (+)ssRNA virus is a picornavirus, an astrovirus, a calicivirus, a hepevirus, a flavivirus, a togavirus, an arterivirus or a coronavirus.

6. The compound for use, method or use according to claim 5, wherein the (+)ssRNA virus is a coronavirus.

7. The compound for use, method or use according to claim 5 or 6, wherein the coronavirus is an Alphacoronavirus, a Betacoronavirus, a Gammacoronavirus or a Deltacoronavirus.

8. The compound for use, method or use according to any one of claims 5 to 7, wherein the coronavirus is selected from the group consisting of SARS-CoV, SARS-CoV-2, MERS-CoV, HCoV-NL63, HCoV-229E, HCov-OC43 and HKU1.

9. The compound for use, method or use according to claim 8, wherein the coronavirus is SARS-CoV, SARS-CoV-2 or MERS-CoV.

10. The compound for use, method or use according to claim 9, wherein the coronavirus is SARS-CoV-2.

11. The compound for use, method or use according to any one of claims 1 to 3, wherein the disease is a disease associated with a positive-sense single-stranded RNA virus ((+)ssRNA virus) infection.

12. The compound for use, method or use according to claim 11, wherein the disease is SARS, MERS or COVID-19.

13. The compound for use, method or use according to claim 12, wherein the disease is COVID-19.

14. The compound for use, method or use according to any one of claims 1 to 11, wherein the disease associated with a positive-sense single-stranded RNA virus ((+)ssRNA virus) infection is a complication induced by the virus, selected from the group consisting of respiratory distress, pulmonary fibrosis, pneumonia, cytokine storm; acute liver injury, septic shock, acute kidney injury, pancreatic injury, peripheral nervous systems complications (such as an impaired ability to taste, to smell, and vision impairment), muscle pain, inflammation of cardiac muscle, blood clots in veins, decreased blood flow in coronary arteries, cardiogenic shock, heart failure, impaired consciousness, brain inflammation, irritation and swelling of brain and blood vessels, acute cerebrovascular complications (such as stroke, seizures and slurred speech), arrhythmia, myocarditis, thrombotic events rhabdomyolysis, neurocognitive deficits, and sensory and motor deficits.

15. The compound for use, method or use according to claim 14, wherein the positive-sense single-stranded RNA virus ((+)ssRNA virus) is SARS-CoV-2.

16. The compound for use, method or use according to any one of claims 1 to 3, wherein the disease is lung inflammation.

17. The compound for use, method or use according to claim 16, wherein the lung inflammation is caused by pathogenic infection, bacterial infection, fungal infection or viral infection, in particular (+)ssRNA virus infection,

18. The compound for use, method or use according to claim 16 or claim 17, wherein the lung inflammation is caused by a disease selected from the group consisting of pneumonia, acute respiratory disease symptom (ARDS), COPD, asthma, idiopathic pulmonary fibrosis, allergic rhinitis, rhinitis and sinusitis.

19. The compound for use, method or use according to claim 18, wherein the lung inflammation is caused by COPD, asthma or idiopathic pulmonary fibrosis.

20. The compound for use, method or use according to claim 19, wherein the lung inflammation is caused by idiopathic pulmonary fibrosis.

21. The compound for use, method or use according to any one of claims 1 to 20, wherein the compound reduces hyperinflammation associated with positive-sense single-stranded RNA virus infection, e.g. coronavirus infection.

22. The compound for use, method or use according to any one of claims 1 to 21, wherein the compound is selected from the compounds of Table 2.

23. The compound for use, method or use according to any one of claims 1 to 21, wherein the compound is selected from the compounds of Table 3.

24. The compound for use, method or use according to any one of claims 1 to 21, wherein the compound is selected from the compounds of Table 4.

25. The compound for use, method or use according to any one of claims 1 to 21, wherein the compound is selected from the compounds of Table 5.

26. A pharmaceutical composition comprising a compound selected from Compound 30 (felodipine) and Compounds 1 to 29 and 31 to 203 and prodrugs thereof and pharmaceutically acceptable salts and solvates thereof; and a pharmaceutically acceptable diluent or carrier, for use in the treatment or prevention of a disease selected from the group consisting of a positive-sense single-stranded RNA virus ((+)ssRNA virus) infection, a disease associated with a positive-sense single-stranded RNA virus ((+)ssRNA virus) infection and lung inflammation.

27. A compound selected from Compound 30 (felodipine) and Compounds 1 to 29 and 31 to 203 and prodrugs thereof and pharmaceutically acceptable salts and solvates thereof for use according to any one of claims 1 to 25 in combination with a second or further compound selected from Compound 30 (felodipine) and Compounds 1 to 29 and 31 to 203 and prodrug thereof and pharmaceutically acceptable salt thereof and solvate thereof for the treatment or prevention of a disease selected from the group consisting of a positive-sense single-stranded RNA virus ((+)ssRNA virus) infection, a disease associated with a positive-sense single-stranded RNA virus ((+)ssRNA virus) infection and lung inflammation.

28. A compound selected from the compounds of category C1, C2, C3, C4 or C5 and prodrugs thereof and pharmaceutically acceptable salts and solvates thereof for use according to any one of claims 1 to 25 in combination with a second or further compound of a different category selected from the compounds of category C1, C2, C3, C4 or C5 and prodrug thereof and pharmaceutically acceptable salt thereof and solvate thereof, provided that at least one compound of the combination is a compound selected from Compound 30 (felodipine) and Compounds 1 to 29 and 31 to 203 and prodrugs thereof and pharmaceutically acceptable salts and solvates thereof, for the treatment or prevention of a disease selected from the group consisting of a positive-sense single-stranded RNA virus ((+)ssRNA virus) infection, a disease associated with a positive-sense single-stranded RNA virus ((+)ssRNA virus) infection and lung inflammation.

29. A compound selected from the compounds of category CC1, CC2, CC3, CC4 or CC5 and prodrugs thereof and pharmaceutically acceptable salts and solvates thereof for use according to any one of claims 1 to 25 in combination with a second or further compound of a different category selected from the compounds of category CC1, CC2, CC3, CC4 or CC5 and prodrug thereof and pharmaceutically acceptable salt thereof and solvate thereof, provided that at least one compound of the combination is a compound selected from Compound 30 (felodipine) and Compounds 1 to 29 and 31 to 203 and prodrugs thereof and pharmaceutically acceptable salts and solvates thereof, for the treatment or prevention of a disease selected from the group consisting of a positive-sense single-stranded RNA virus ((+)ssRNA virus) infection, a disease associated with a positive-sense single-stranded RNA virus ((+)ssRNA virus) infection and lung inflammation.

30. A compound selected from the compounds of category C1, C2, C3, C4 or C5 and prodrugs thereof and pharmaceutically acceptable salts and solvates thereof for use according to any one of claims 1 to 25 in combination with a second or further compound of the same category selected from the compounds of category C1, C2, C3, C4 or C5 and prodrug thereof and pharmaceutically acceptable salt thereof and solvate thereof, provided that at least one compound of the combination is a compound selected from Compound 30 (felodipine) and Compounds 1 to 29 and 31 to 203 and prodrugs thereof and pharmaceutically acceptable salts and solvates thereof, for the treatment or prevention of a disease selected from the group consisting of a positive-sense single-stranded RNA virus ((+)ssRNA virus) infection, a disease associated with a positive-sense single-stranded RNA virus ((+)ssRNA virus) infection and lung inflammation.

31. A compound selected from the compounds of category CC1, CC2, CC3, CC4 or CC5 and prodrugs thereof and pharmaceutically acceptable salts and solvates thereof for use according to any one of claims 1 to 25 in combination with a second or further compound of the same category selected from the compounds of category CC1, CC2, CC3, CC4 or CC5 and prodrug thereof and pharmaceutically acceptable salt thereof and solvate thereof, provided that at least one compound of the combination is a compound selected from Compound 30 (felodipine) and Compounds 1 to 29 and 31 to 203 and prodrugs thereof and pharmaceutically acceptable salts and solvates thereof, for the treatment or prevention of a disease selected from the group consisting of a positive-sense single-stranded RNA virus ((+)ssRNA virus) infection, a disease associated with a positive-sense single-stranded RNA virus ((+)ssRNA virus) infection and lung inflammation.

32. A compound selected from Compound 30 (felodipine) and Compounds 1 to 29 and 31 to 203 and prodrugs thereof and pharmaceutically acceptable salts and solvates thereof for use according to any one of claims 1 to 25 in combination with a second or further compound selected from chloroquine, tacrolimus, thalidomide, dexamethasone and sirolimus for the treatment or prevention of a disease selected from the group consisting of a positive-sense single-stranded RNA virus ((+)ssRNA virus) infection, a disease associated with a positive-sense single-stranded RNA virus ((+)ssRNA virus) infection and lung inflammation.

33. The compound for use according to any one of claims 28 to 32 wherein the compound selected Compound 30 (felodipine) and Compounds 1 to 29 and 31 to 203 is selected from Table 2.

34. The compound for use according to any one of claims 28 to 32 wherein the compound selected from Compound 30 (felodipine) and Compounds 1 to 29 and 31 to 203 is selected from Table 3.

35. The compound for use according to any one of claims 28 to 32 wherein the compound selected from Compound 30 (felodipine) and Compounds 1 to 29 and 31 to 203 is selected from Table 4.

36. The compound for use according to any one of claims 28 to 32 wherein the compound selected from Compound 30 (felodipine) and Compounds 1 to 29 and 31 to 203 is selected from Table 5.

37. The compound for use according to any one of claims 28 to 32 wherein the compound selected from Compound 30 (felodipine) and Compounds 1 to 29 and 31 to 203 is selected from the group consisting of PD-0325901, sulforaphane, neratinib, curcumin and trifluoperazine.

38. A compound selected from Compounds 1 to 203 and prodrugs thereof and pharmaceutically acceptable salts and solvates thereof for use in combination with a second or further compound selected from Compounds 1 to 203 and prodrugs thereof and pharmaceutically acceptable salts and solvates thereof for the treatment or prevention of a disease selected from the group consisting of a positive-sense single-stranded RNA virus ((+)ssRNA virus) infection, a disease associated with a positive-sense single-stranded RNA virus ((+)ssRNA virus) infection and lung inflammation, and wherein the first and the second or further compounds are selected from the group consisting of calcium channel blockers, antihistamines (histamine receptor antagonist), statins, glucocorticoids and anti-psychotics.

39. The compound for use according to claim 38, wherein the first compound is fluvoxamine and the second or further compound is selected from nifedipine, felodipine, desloratadine, promethazine and atorvastatin.

40. The compound for use according to claim 38, wherein the first compound is nifedipine and the second or further compound is promethazine.

41. The compound for use according to claim 38, wherein the first compound is felodipine, and the second or further compound is selected from promethazine, desloratadine and fluvoxamine.

42. The compound for use according to claim 38, wherein the first compound is atorvastatin and the second or further compound is selected from nifedipine, felodipine, desloratadine, promethazine and fluvoxamine.

43. The compound for use according to claim 38, wherein the first compound is hydrocortisone and the second or further compound is selected from fluvoxamine, nifedipine, felodipine, desloratadine, promethazine and atorvastatin.
